SEQUENCE LISTING (1 sequence)

SEQ ID NO: 1 - protein, 17 aa, Homo sapiens
CDDYYYGFGCNKFCRPR

(19) United States
(12) Patent Application Publication
(10) Pub. No.: US 2024/0294713 A1
(43) Pub. Date: Sep. 5, 2024

(54) THERMOREVERSIBLE POLYMERS AND METHODS OF USE THEREOF

(71) Applicant: The Regents of the University of California, Oakland, CA (US)

(72) Inventors: David V. Schaffer, Danville, CA (US); Hunter Johnson, Berkeley, CA (US)

(21) Appl. No.: 18/289,733

(22) PCT Filed: May 23, 2022
(86) PCT No.: PCT/US22/30591

§ 371 Date: Nov. 6, 2023

Related U.S. Application Data
(60) Provisional application No. 63/192,311, filed on May 24, 2021.

(51) Int. Cl. C08G 81/02; C12N 5/074

(52) U.S. Cl. CPC C08G 81/025; C12N 5/0696; C08G 2210/00; C12N 2533/30

(57) ABSTRACT

The present disclosure provides thermoreversible polymers, hydrogel compositions comprising the thermoreversible polymers, as well as methods of making and using the thermoreversible polymers. In some cases, the thermoreversible polymer comprises a N-isopropylacrylamide (NIPAM) co-monomer, a lower alkyl amine co-monomer and a polyethylene glycol) (PEG) comonomer.

### CROSS-REFERENCE

This application claims the benefit of U.S. Provisional Patent Application No. 63/192,311, filed May 24, 2021, which application is incorporated herein by reference in its entirety.

### INTRODUCTION

Patients who suffer from a broad range of disorders involving tissue degeneration—such as Parkinson's disease, a myocardial infarction (heart attack), or liver failure—could potentially benefit from implantation of new healthy cells or engineered tissues to replace damaged or diseased ones, a process known as cell replacement therapy. Stem cells have the unique abilities to replicate indefinitely in an immature state and to differentiate into various types of cells found in the body. Therefore, stem cells can be harnessed as the cell source for such cell replacement and tissue engineering therapies. As such, systems and methods for scalable stem cell expansion and differentiation are of interest.

### SUMMARY

The present disclosure provides thermoreversible polymers, hydrogel compositions comprising the thermoreversible polymers, as well as methods of making and using the thermoreversible polymers.

### BRIEF DESCRIPTION OF THE DRAWINGS

FIG. 1 is a schematic of synthesis of a thermoreversible polymer of the present disclosure.

FIG. 2A-2B depict structural characterization of a thermoreversible polymer of the present disclosure.

FIG. 3A-3G depict mechanical properties of thermoreversible polymers of the present disclosure.

FIG. 4A-4D depict reproducibility and scalability of a thermoreversible polymer of the present disclosure.

FIG. 5A-5B depict functionalization of a thermoreversible polymer of the present disclosure.

FIG. 6A-6E depict human pluripotent stem cell (hPSC) viability and expansion in a biomaterial comprising a thermoreversible polymer of the present disclosure.

FIG. 7A-7I depict differentiation of hPSCs in a hydrogel of the present disclosure or in Matrigel.

FIG. 8 is a schematic of synthesis of a thermoreversible polymer (graft copolymer; GCP) of the present disclosure, using butyl methacrylate (BMA).

FIG. 9A-9C schematically depict functionalization of a GCP.

FIG. 10A-10D depict rheology analysis of functionalized GCPs.

FIG. 11A-11D schematically depict functionalization of a GCP.

FIG. 12 schematically depicts GCP-RAFT synthesis.

FIG. 13A-13C present data showing the mechanical properties of GCP-BMA.

### DEFINITIONS

The following terms have the following meanings unless otherwise indicated. Any undefined terms have their art recognized meanings.

The term “cell culture” or “culturing of cells” refers to maintaining, transporting, isolating, culturing, propagating, passaging or differentiating of cells or tissues. Cells can be in any arrangement such as individual cells, monolayers, cell clusters or spheroids or as tissue.

As used herein, the term “linker” or “linkage” refers to a linking moiety that connects two groups and has a backbone of 100 atoms or less in length. A linker or linkage may be a covalent bond that connects two groups or a chain of between 1 and 100 atoms in length, for example of 1, 2, 3, 4, 5, 6, 8, 10, 12, 14, 16, 18 or 20 carbon atoms in length, where the linker may be linear, branched, cyclic or a single atom. In certain cases, one, two, three, four or five or more carbon atoms of a linker backbone may be optionally substituted with a sulfur, nitrogen or oxygen heteroatom. The bonds between backbone atoms may be saturated or unsaturated, usually not more than one, two, or three unsaturated bonds will be present in a linker backbone. The linker may include one or more substituent groups, for example with an alkyl, aryl or alkenyl group. A linker may include, without limitations, poly(ethylene glycol); ethers, thioethers, tertiary amines, alkyls, which may be straight or branched, e.g., methyl, ethyl, n-propyl, 1-methylethyl (iso-propyl), n-butyl, n-pentyl, 1,1-dimethylethyl (t-butyl), and the like. The linker backbone may include a cyclic group, for example, an aryl, a heterocycle or a cycloalkyl group, where 2 or more atoms, e.g., 2, 3 or 4 atoms, of the cyclic group are included in the backbone. A linker may be cleavable or non-cleavable.

“Alkyl” refers to monovalent saturated aliphatic hydrocarbyl groups having from 1 to 10 carbon atoms and such as 1 to 6 carbon atoms, or 1 to 5, or 1 to 4, or 1 to 3 carbon atoms. In some cases, a “lower alkyl” is an alkyl group having 1 to 6 carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—).

The term “substituted alkyl” refers to an alkyl group as defined herein wherein one or more carbon atoms in the alkyl chain have been optionally replaced with a heteroatom such as —O—, —N—, —S—, —S(O)ₙ— (where n is 0 to 2), —NR— (where R is hydrogen or alkyl) and having from 1 to 5 substituents selected from the group consisting of alkoxy, substituted alkoxy, cycloalkyl, substituted cycloalkyl, cycloalkenyl, substituted cycloalkenyl, acyl, acylamino, acyloxy, amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-aryl, —SO-heteroaryl, —SO₂-alkyl, —SO₂-aryl, —SO₂-heteroaryl, and —NR^{a}R^{b}, wherein R′ and R″ may be the same or different and are chosen from hydrogen, optionally substituted alkyl, cycloalkyl, alkenyl, cycloalkenyl, alkynyl, aryl, heteroaryl and heterocyclic.

As used herein, the terms “chemoselective functional group” and “chemoselective tag” are used interchangeably and refer to chemoselective reactive groups that selectively react with one another to form a covalent bond. Chemoselective functional groups of interest include, but are not limited to, two thiol groups, thiols and maleimide or iodoacetamide, as well as groups that can react with one another via Click chemistry, e.g., azide and alkyne groups (e.g., cyclooctyne groups). Chemoselective functional groups of interest, include, but are not limited to, thiols, alkyne, a cyclooctyne, an azide, a phosphine, a maleimide, an alkoxyamine, an aldehyde and protected versions thereof, and percursors thereof. In certain embodiments, the chemoselective functional group is a thiol. In some cases, a chemoselective functional group is a strained alkyne such as dibenzocyclooctyne (DBCO).

The term “RAFT” is used herein in its conventional sense to refer to reversible addition-fragmentation chain transfer polymerization. In some embodiments, RAFT agents (or chain-transfer agents) for use in the methods and in preparing the compounds described herein include, but are not limited to dithioesters, dithiocarbamates, trithiocarbonates and xanthates. In some cases, the RAFT agent is a dithiobenzoate having a structure of:

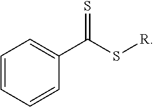

In some cases, the RAFT agent is a trithiocarbonate having a structure of:

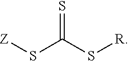

In some cases, the RAFT agent is a dithiocarbamate having a structure of:

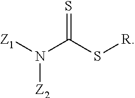

As used, herein the lower critical solution temperature (LCST) or lower consolute temperature refers to the critical temperature below which the components of a mixture are miscible for all compositions. The word lower in the term indicates that the LCST is a lower bound to a temperature interval of partial miscibility, or miscibility for certain compositions only.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a thermoreversible polymer” includes a plurality of such polymers and reference to “the hydrogel composition” includes reference to one or more hydrogel compositions and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides thermoreversible polymers, hydrogel compositions comprising the thermoreversible polymers, as well as methods of making and using the thermoreversible polymers.

### Thermoreversible Polymers

Aspects of the present disclosure include thermoreversible polymers (also referred to as “thermosensitive polymers” or “thermoresponsive polymers”). As used herein, the term “thermoreversible” is used to refer to a polymeric material that exhibits a drastic change in its physical property with a change in temperature. Thermoreversible polymers belong to the class of stimuli-responsive materials. In some cases, a thermoreversible polymer is distinguished from a temperature-sensitive (e.g., thermosensitive) material, which can change physical properties continuously with environmental conditions. A thermoresponsive polymer can display a miscibility gap in its temperature-composition diagram. Depending on whether the miscibility gap is found at high or low temperatures, an upper or lower critical solution temperature exists, respectively (abbreviated UCST or LCST, respectively). For example, at a temperature below the LCST, a thermoresponsive polymer can be miscible with an aqueous solution in which it dissolves. At a temperature above the LCST, the thermoresponsive polymer forms a solid, semi-solid, or gel having a three-dimensional (3D) structure.

In some cases, the thermoreversible polymer comprises a N-isopropylacrylamide (NIPAM) co-monomer, a lower alkyl amine co-monomer and a poly(ethylene glycol) (PEG) co-monomer, wherein the terminal PEG monomer is substituted with alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl. In certain embodiments, the lower alkyl amine co-monomer comprises n-butyl, isobutyl, tert-butyl, n-propyl, pentyl, isopropyl, or isopentyl and the terminal PEG monomer is substituted with an alkoxy group. In some instances, the alkoxy group is an C1-C6 alkoxy selected from the group consisting of methoxy, ethoxy, n-propoxy, isoproxy, n-butoxy, isobutoxy, tert-butoxy, pentoxy and isopentoxy.

In embodiments of the present disclosure, thermoreversible polymers (e.g., as described in greater detail below) may have a molecular weight which varies, such as from 5 kDa to 750 kDa, such as from 10 kDa to 500 kDa, such as from 15 kDa to 450 kDa, such as from 20 kDa to 400 kDa, such as from 25 kDa to 350 kDa, such as from 30 kDa to 300 kDa, such as from 35 kDa to 250 kDa and including from 40 kDa to 200 kDa. In certain embodiments, thermoreversible polymers of interest have a molecular weight of from 10 kDa to 500 kDa.

In some cases, the thermoreversible polymer is a polymer of formula (I):

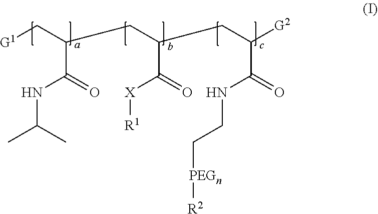

wherein:
a, b, and c are molar fractions of the co-monomers, where a, b, and c are each greater than zero;
PEGₙ is a polyethyleneglycol polymer and n is an integer from 1 to 2500;
X is independently selected from C, O, and NH. In some instances, X is O. In some instances, X is NH.
R¹ is an alkyl or a substituted alkyl;
R² is alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl; and
G¹ and G² are each independently selected from a polymer segment, a terminal group, a linker and a linked modifying agent. In some embodiments, G¹ and G² are each independently a chain-transfer agent. For example, the chain-transfer agent may be a dithioester, dithiocarbamate, trithiocarbonate or a xanthate. In certain embodiments, G¹ is a carboxyl group. In certain instances, G¹ is:

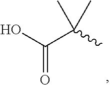

wherein [symbol] is a bond between G¹ and the polymer. In certain embodiments, G² is:

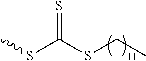

wherein [symbol] is a bond between G² and the polymer.

In some cases, n is an integer from 1-25, 1-50, 1-100, 25-100, 50-100, 1-150, 25-150, 50-150, 100-150, 100-125, 100-150, 150-200, 200-250, 250-500, 500-1000, 1000-1500, 1500-2000, or 2000-2500. In some embodiments, the thermoreversible polymer has a molecular weight which varies, such as from 5 kDa to 750 kDa, such as from 10 kDa to 500 kDa, such as from 15 kDa to 450 kDa, such as from 20 kDa to 400 kDa, such as from 25 kDa to 350 kDa, such as from 30 kDa to 300 kDa, such as from 35 kDa to 250 kDa and including from 40 kDa to 200 kDa. In certain embodiments, the thermoreversible polymer has a molecular weight of from 10 kDa to 500 kDa.

In some embodiments, R¹ is a C1-C6 alkyl. In some instances, R1 is an alkyl group selected from the group consisting of n-butyl, isobutyl, tert-butyl, n-propyl, pentyl, isopropyl and isopentyl. In certain instances, R¹ is n-butyl.

In some embodiments, R² is an alkoxy group. In some instances, R² is a C1-C6 alkoxy group selected from the group consisting of methoxy, ethoxy, n-propoxy, isoproxy, n-butoxy, isobutoxy, tert-butoxy, pentoxy and isopentoxy. In certain instances, R² is methoxy.

In certain instances, a>0.8; 0.2>b>0; and 0.1>c>0.

In certain embodiments, the thermoreversible polymer is a polymer of formula (II):

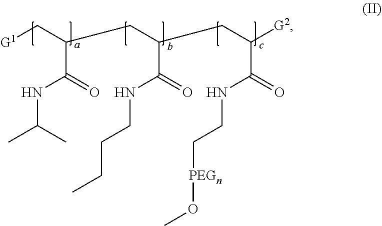

wherein n is 1 to 2500; and
G¹ and G² are each independently selected from a polymer segment, a terminal group, a linker and a linked modifying agent. In some embodiments, G¹ and G² are each independently a chain-transfer agent. For example, the chain-transfer agent may be a dithioester, dithiocarbamate, trithiocarbonate or a xanthate. In certain embodiments, G¹ is a carboxyl group. In certain instances, G¹ is:

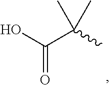

wherein [symbol] is a bond between G¹ and the polymer. In certain embodiments, G² is:

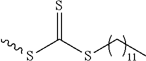

wherein [symbol] is a bond between G² and the polymer.

In certain embodiments, the PEG or PEGₙ has a MW of 2 kDa to 100 kDa.

In some cases, n is an integer from 1-25, 1-50, 1-100, 25-100, 50-100, 1-150, 25-150, 50-150, 100-150, 100-125, 100-150, 150-200, 200-250, 250-500, 500-1000, 1000-1500, 1500-2000, or 2000-2500. In some embodiments, the thermoreversible polymer has a molecular weight which varies, such as from 5 kDa to 750 kDa, such as from 10 kDa to 500 kDa, such as from 15 kDa to 450 kDa, such as from 20 kDa to 400 kDa, such as from 25 kDa to 350 kDa, such as from 30 kDa to 300 kDa, such as from 35 kDa to 250 kDa and including from 40 kDa to 200 kDa. In certain embodiments, the thermoreversible polymer has a molecular weight of from 10 kDa to 500 kDa.

In some embodiments, the thermoreversible polymer is a polymer of formula (III):

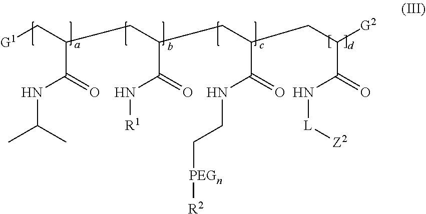

wherein:
a, b, c, and d are molar fractions of the co-monomers, where a, b, c and d are each greater than zero;
PEGₙ is a polyethyleneglycol polymer and n is an integer from 1 to 2500;
R¹ is an alkyl or a substituted alkyl;
R² is alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl;
L is a linker;
Z² is a modifying agent or a chemoselective functional group; and
G¹ and G² are each independently selected from a polymer segment, a terminal group, a linker and a linked modifying agent. In some embodiments, G¹ and G² are each independently a chain-transfer agent. For example, the chain-transfer agent may be a dithioester, dithiocarbamate, trithiocarbonate or a xanthate. In certain embodiments, G¹ is a carboxyl group. In certain instances, G¹ is:

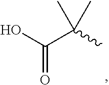

wherein [symbol] is a bond between G¹ and the polymer. In certain embodiments, G² is:

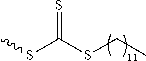

wherein [symbol] is a bond between G² and the polymer.

In some cases, n is an integer from 1-25, 1-50, 1-100, 25-100, 50-100, 1-150, 25-150, 50-150, 100-150, 100-125, 100-150, 150-200, 200-250, 250-500, 500-1000, 1000-1500, 1500-2000, or 2000-2500. In some embodiments, the thermoreversible polymer has a molecular weight which varies, such as from 5 kDa to 750 kDa, such as from 10 kDa to 500 kDa, such as from 15 kDa to 450 kDa, such as from 20 kDa to 400 kDa, such as from 25 kDa to 350 kDa, such as from 30 kDa to 300 kDa, such as from 35 kDa to 250 kDa and including from 40 kDa to 200 kDa. In certain embodiments, the thermoreversible polymer has a molecular weight of from 10 kDa to 500 kDa.

In some embodiments, R¹ is a C1-C6 alkyl. In some instances, R1 is an alkyl group selected from the group consisting of n-butyl, isobutyl, tert-butyl, n-propyl, pentyl, isopropyl and isopentyl. In certain instances, R¹ is n-butyl.

In some embodiments, R² is an alkoxy group. In some instances, R² is a C1-C6 alkoxy group selected from the group consisting of methoxy, ethoxy, n-propoxy, isoproxy, n-butoxy, isobutoxy, tert-butoxy, pentoxy and isopentoxy. In certain instances, R² is methoxy.

In certain instances, a>0.8; 0.2>b>0; 0.1>c>0, and 0.1>d>0.

In some embodiments, Z² is a chemoselective functional group selected from a thiol, an alkyne, a cyclooctyne, an azide, a phosphine, a maleimide, an alkoxyamine, an aldehyde, and protected versions or precursors thereof.

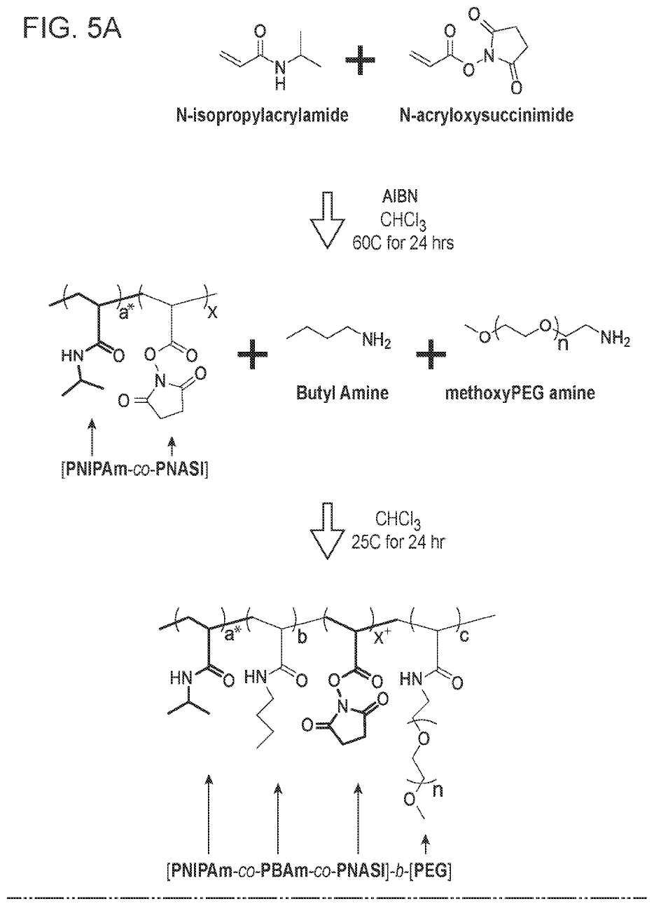

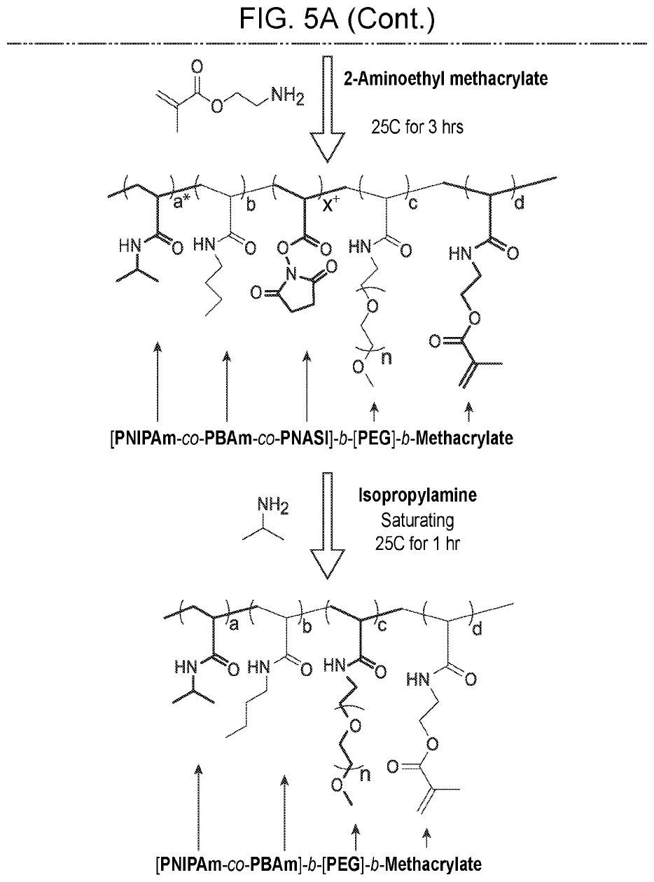

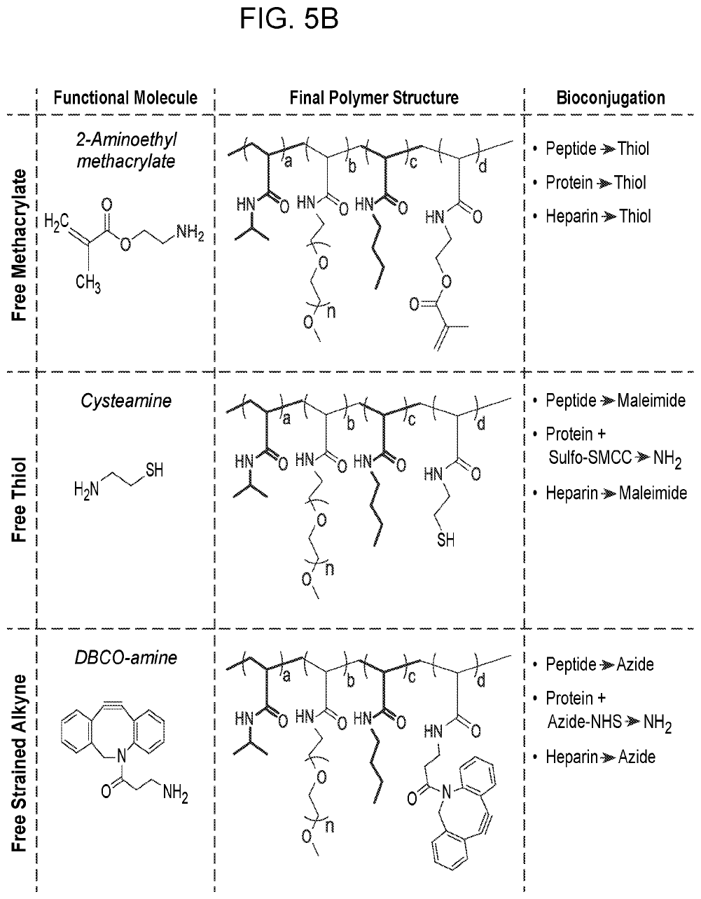

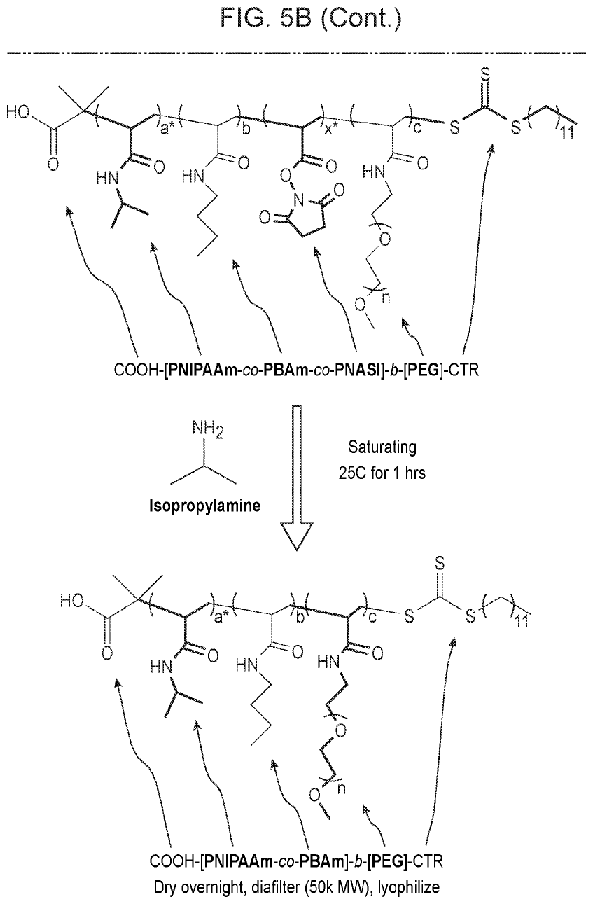

In some cases, Z² comprises a group such as a methacrylate. As depicted in FIG. 5B, such a group can be used to attach thiol-containing molecules. For example, thiol-containing molecules can be proteins, peptides, heparin, and the like that either contain a free thiol group or are modified to contain a free thiol group. In some cases, Z² comprises a thiol. As depicted in FIG. 5B, a free thiol can be used as the attachment point for a variety of molecules, using standard chemistries. In some cases, Z² comprises a strained alkyne. As depicted in FIG. 5B, a strained alkyne can be used to attach a variety of molecules, using well known reactions.

In some instances, Z² is a modifying agent selected from a heparin, a hyaluronic acid, a specific binding member, a peptide, a fibroblast growth factor (FGF), a nucleic acid, gelatin, fibronectin, collagen, laminin, basic fibroblast growth factor (bFGF; also known as fibroblast growth factor 2 (FGF2)), FGF7, FGF8, FGF10, epidermal growth factor (EGF), insulin, progesterone, glucose, stromal cell derived factor-1 (SDF-1), thymosin beta-4, sonic hedgehog (SHH), Noggin, Activin, transforming growth factor-β (TGF-β) (TGFβ3), brain-derived neurotrophic factor (BDNF), glial cell-derived neurotrophic factor (GDNF), neurotrophic factor-3 (NT3), nerve growth factor (NGF), platelet-derived growth factor (PDGF), an interleukin (e.g., IL-2 or IL-16) and insulin-like growth factor-1 (IGF-1).

In some instances, one or more of G¹, G² and Z² are independently selected a modifying agent selected from a heparin, a hyaluronic acid, a member of a specific binding pair, a polypeptide, and a nucleic acid. In some instances, one or more of G¹, G² and Z² are independently selected a modifying agent selected from gelatin, fibronectin, collagen, or laminin.

In some instances, one or more of G¹, G² and Z² is a polypeptide selected from a chemokine, a peptide hormone, or a growth factor. In certain instances, the polypeptide is fibroblast growth factor, epidermal growth factor, hepatocyte growth factor, insulin, stromal cell-derived factor-1, thymosin beta-4, sonic hedgehog, Noggin, activin, transforming growth factor, bone morphogenic protein, brain-derived neurotrophic factor, glial cell-derived neurotrophic factor, neurotrophin-3, platelet-derived growth factor, FGF-2, FGF-8, keratinocyte growth factor or insulin-like growth factor. In some instances, the polypeptide is selected from hepatocyte growth factor; bone morphogenic protein; FGF-2; FGF-8; and keratinocyte growth factor.

In some embodiments, the thermoreversible polymer is a polymer of formula (IV):

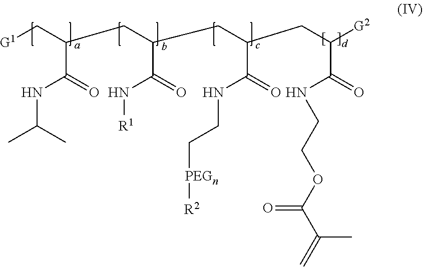

wherein:
a, b, c, and d are molar fractions of the co-monomers, where a, b, c and d are each greater than zero;
PEGₙ is a polyethyleneglycol polymer and n is an integer from 1 to 2500;
R¹ is an alkyl or a substituted alkyl;
R² is alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl;
G¹ and G² are each independently selected from a polymer segment, a terminal group, a linker and a linked modifying agent. In some embodiments, G¹ and G² are each independently a chain-transfer agent. For example, the chain-transfer agent may be a dithioester, dithiocarbamate, trithiocarbonate or a xanthate. In certain embodiments, G¹ is a carboxyl group. In certain instances, G¹ is:

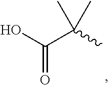

wherein [symbol] is a bond between G¹ and the polymer. In certain embodiments, G² is:

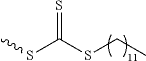

wherein [symbol] is a bond between G² and the polymer.

In some cases, n is an integer from 1-25, 1-50, 1-100, 25-100, 50-100, 1-150, 25-150, 50-150, 100-150, 100-125, 100-150, 150-200, 200-250, 250-500, 500-1000, 1000-1500, 1500-2000, or 2000-2500. In some embodiments, the thermoreversible polymer has a molecular weight which varies, such as from 5 kDa to 750 kDa, such as from 10 kDa to 500 kDa, such as from 15 kDa to 450 kDa, such as from 20 kDa to 400 kDa, such as from 25 kDa to 350 kDa, such as from 30 kDa to 300 kDa, such as from 35 kDa to 250 kDa and including from 40 kDa to 200 kDa. In certain embodiments, the thermoreversible polymer has a molecular weight of from 10 kDa to 500 kDa.

In some embodiments, R¹ is a C1-C6 alkyl. In some instances, R1 is an alkyl group selected from the group consisting of n-butyl, isobutyl, tert-butyl, n-propyl, pentyl, isopropyl and isopentyl. In certain instances, R¹ is n-butyl.

In some embodiments, R² is an alkoxy group. In some instances, R² is a C1-C6 alkoxy group selected from the group consisting of methoxy, ethoxy, n-propoxy, isoproxy, n-butoxy, isobutoxy, tert-butoxy, pentoxy and isopentoxy. In certain instances, R² is methoxy.

In certain instances, a>0.8; 0.2>b>0; 0.1>c>0, and 0.1>d>0.

In some embodiments, the thermoreversible polymer is a polymer of formula (V):

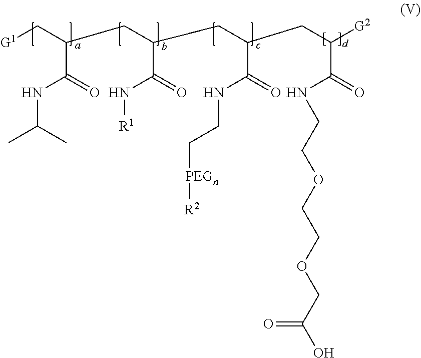

wherein:
a, b, c, and d are molar fractions of the co-monomers, where a, b, c and d are each greater than zero;
PEGₙ is a polyethyleneglycol polymer and n is an integer from 1 to 2500;
R¹ is an alkyl or a substituted alkyl;
R² is alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl;

G¹ and G² are each independently selected from a polymer segment, a terminal group, a linker and a linked modifying agent. In some embodiments, G¹ and G² are each independently a chain-transfer agent. For example, the chain-transfer agent may be a dithioester, dithiocarbamate, trithiocarbonate or a xanthate. In certain embodiments, G¹ is a carboxyl group. In certain instances, G¹ is:

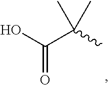

wherein [symbol] is a bond between G¹ and the polymer. In certain embodiments, G² is:

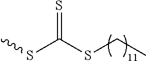

wherein [symbol] is a bond between G² and the polymer.

In some cases, n is an integer from 1-25, 1-50, 1-100, 25-100, 50-100, 1-150, 25-150, 50-150, 100-150, 100-125, 100-150, 150-200, 200-250, 250-500, 500-1000, 1000-1500, 1500-2000, or 2000-2500. In some embodiments, the thermoreversible polymer has a molecular weight which varies, such as from 5 kDa to 750 kDa, such as from 10 kDa to 500 kDa, such as from 15 kDa to 450 kDa, such as from 20 kDa to 400 kDa, such as from 25 kDa to 350 kDa, such as from 30 kDa to 300 kDa, such as from 35 kDa to 250 kDa and including from 40 kDa to 200 kDa. In certain embodiments, the thermoreversible polymer has a molecular weight of from 10 kDa to 500 kDa.

In some embodiments, R¹ is a C1-C6 alkyl. In some instances, R1 is an alkyl group selected from the group consisting of n-butyl, isobutyl, tert-butyl, n-propyl, pentyl, isopropyl and isopentyl. In certain instances, R¹ is n-butyl.

In some embodiments, R² is an alkoxy group. In some instances, R² is a C1-C6 alkoxy group selected from the group consisting of methoxy, ethoxy, n-propoxy, isoproxy, n-butoxy, isobutoxy, tert-butoxy, pentoxy and isopentoxy. In certain instances, R² is methoxy.

In certain instances, a>0.8; 0.2>b>0; 0.1>c>0, and 0.1>d>0.

In some embodiments, the thermoreversible polymer is a polymer of formula (VI):

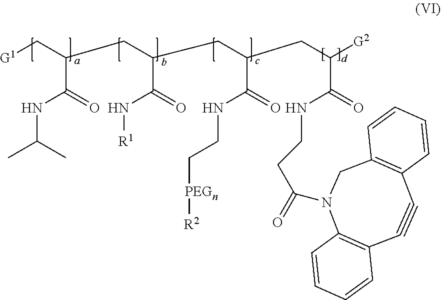

wherein:
a, b, c, and d are molar fractions of the co-monomers, where a, b, c and d are each greater than zero;
PEGₙ is a polyethyleneglycol polymer and n is an integer from 1 to 2500;
R¹ is an alkyl or a substituted alkyl;
R² is alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl;
G¹ and G² are each independently selected from a polymer segment, a terminal group, a linker and a linked modifying agent. In some embodiments, G¹ and G² are each independently a chain-transfer agent. For example, the chain-transfer agent may be a dithioester, dithiocarbamate, trithiocarbonate or a xanthate. In certain embodiments, G¹ is a carboxyl group. In certain instances, G¹ is:

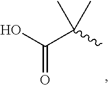

wherein [symbol] is a bond between G¹ and the polymer. In certain embodiments, G² is:

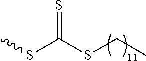

wherein [symbol] is a bond between G² and the polymer.

In some cases, n is an integer from 1-25, 1-50, 1-100, 25-100, 50-100, 1-150, 25-150, 50-150, 100-150, 100-125, 100-150, 150-200, 200-250, 250-500, 500-1000, 1000-1500, 1500-2000, or 2000-2500. In some embodiments, the thermoreversible polymer has a molecular weight which varies, such as from 5 kDa to 750 kDa, such as from 10 kDa to 500 kDa, such as from 15 kDa to 450 kDa, such as from 20 kDa to 400 kDa, such as from 25 kDa to 350 kDa, such as from 30 kDa to 300 kDa, such as from 35 kDa to 250 kDa and including from 40 kDa to 200 kDa. In certain embodiments, the thermoreversible polymer has a molecular weight of from 10 kDa to 500 kDa.

In some embodiments, R¹ is a C1-C6 alkyl. In some instances, R1 is an alkyl group selected from the group consisting of n-butyl, isobutyl, tert-butyl, n-propyl, pentyl, isopropyl and isopentyl. In certain instances, R¹ is n-butyl.

In some embodiments, R² is an alkoxy group. In some instances, R² is a C1-C6 alkoxy group selected from the group consisting of methoxy, ethoxy, n-propoxy, isoproxy, n-butoxy, isobutoxy, tert-butoxy, pentoxy and isopentoxy. In certain instances, R² is methoxy.

In certain instances, a>0.8; 0.2>b>0; 0.1>c>0, and 0.1>d>0.

In some embodiments, the thermoreversible polymer is a polymer of formula (VII):

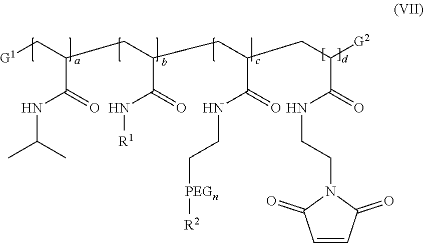

wherein:
a, b, c, and d are molar fractions of the co-monomers, where a, b, c and d are each greater than zero;
PEGₙ is a polyethyleneglycol polymer and n is an integer from 1 to 2500;
R¹ is an alkyl or a substituted alkyl;
R² is alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl;
G¹ and G² are each independently selected from a polymer segment, a terminal group, a linker and a linked modifying agent. In some embodiments, G¹ and G² are each independently a chain-transfer agent. For example, the chain-transfer agent may be a dithioester, dithiocarbamate, trithiocarbonate or a xanthate. In certain embodiments, G¹ is a carboxyl group. In certain instances, G¹ is:

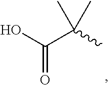

wherein [symbol] is a bond between G¹ and the polymer. In certain embodiments, G² is:

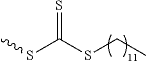

wherein [symbol] is a bond between G² and the polymer.

In some cases, n is an integer from 1-25, 1-50, 1-100, 25-100, 50-100, 1-150, 25-150, 50-150, 100-150, 100-125, 100-150, 150-200, 200-250, 250-500, 500-1000, 1000-1500, 1500-2000, or 2000-2500. In some embodiments, the thermoreversible polymer has a molecular weight which varies, such as from 5 kDa to 750 kDa, such as from 10 kDa to 500 kDa, such as from 15 kDa to 450 kDa, such as from 20 kDa to 400 kDa, such as from 25 kDa to 350 kDa, such as from 30 kDa to 300 kDa, such as from 35 kDa to 250 kDa and including from 40 kDa to 200 kDa. In certain embodiments, the thermoreversible polymer has a molecular weight of from 10 kDa to 500 kDa.

In some embodiments, R¹ is a C1-C6 alkyl. In some instances, R1 is an alkyl group selected from the group consisting of n-butyl, isobutyl, tert-butyl, n-propyl, pentyl, isopropyl and isopentyl. In certain instances, R¹ is n-butyl.

In some embodiments, R² is an alkoxy group. In some instances, R² is a C1-C6 alkoxy group selected from the group consisting of methoxy, ethoxy, n-propoxy, isoproxy, n-butoxy, isobutoxy, tert-butoxy, pentoxy and isopentoxy. In certain instances, R² is methoxy.

In certain instances, a>0.8; 0.2>b>0; 0.1>c>0, and 0.1>d>0.

In some embodiments, the thermoreversible polymer is a polymer of formula (VIII):

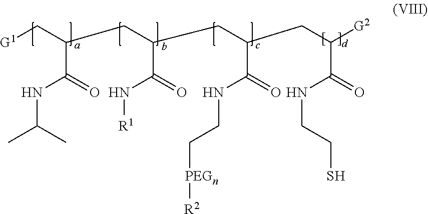

wherein:
a, b, c, and d are molar fractions of the co-monomers, where a, b, c and d are each greater than zero;
PEGₙ is a polyethyleneglycol polymer and n is an integer from 1 to 2500;
R¹ is an alkyl or a substituted alkyl;
R² is alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl;
G¹ and G² are each independently selected from a polymer segment, a terminal group, a linker and a linked modifying agent. In some embodiments, G¹ and G² are each independently a chain-transfer agent. For example, the chain-transfer agent may be a dithioester, dithiocarbamate, trithiocarbonate or a xanthate. In certain embodiments, G¹ is a carboxyl group. In certain instances, G¹ is:

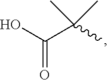

wherein [symbol] is a bond between G¹ and the polymer. In certain embodiments, G² is:

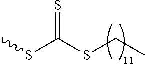

wherein [symbol] is a bond between G² and the polymer.

In some cases, n is an integer from 1-25, 1-50, 1-100, 25-100, 50-100, 1-150, 25-150, 50-150, 100-150, 100-125, 100-150, 150-200, 200-250, 250-500, 500-1000, 1000-1500, 1500-2000, or 2000-2500. In some embodiments, the thermoreversible polymer has a molecular weight which varies, such as from 5 kDa to 750 kDa, such as from 10 kDa to 500 kDa, such as from 15 kDa to 450 kDa, such as from 20 kDa to 400 kDa, such as from 25 kDa to 350 kDa, such as from 30 kDa to 300 kDa, such as from 35 kDa to 250 kDa and including from 40 kDa to 200 kDa. In certain embodiments, the thermoreversible polymer has a molecular weight of from 10 kDa to 500 kDa.

In some embodiments, R¹ is a C1-C6 alkyl. In some instances, R1 is an alkyl group selected from the group consisting of n-butyl, isobutyl, tert-butyl, n-propyl, pentyl, isopropyl and isopentyl. In certain instances, R¹ is n-butyl.

In some embodiments, R² is an alkoxy group. In some instances, R² is a C1-C6 alkoxy group selected from the group consisting of methoxy, ethoxy, n-propoxy, isoproxy, n-butoxy, isobutoxy, tert-butoxy, pentoxy and isopentoxy. In certain instances, R² is methoxy.

In certain instances, a>0.8; 0.2>b>0; 0.1>c>0, and 0.1>d>0.

In some embodiments, the thermoreversible polymer is a polymer of formula (IX):

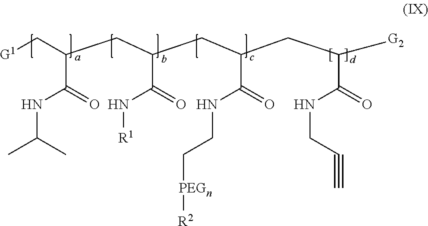

wherein:
a, b, c, and d are molar fractions of the co-monomers, where a, b, c and d are each greater than zero;
PEGₙ is a polyethyleneglycol polymer and n is an integer from 1 to 2500;
R¹ is an alkyl or a substituted alkyl;
R² is alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl;
G¹ and G² are each independently selected from a polymer segment, a terminal group, a linker and a linked modifying agent. In some embodiments, G¹ and G² are each independently a chain-transfer agent. For example, the chain-transfer agent may be a dithioester, dithiocarbamate, trithiocarbonate or a xanthate. In certain embodiments, G¹ is a carboxyl group. In certain instances, G¹ is:

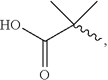

wherein [symbol] is a bond between G¹ and the polymer. In certain embodiments, G² is:

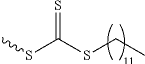

wherein [symbol] is a bond between G² and the polymer.

In some cases, n is an integer from 1-25, 1-50, 1-100, 25-100, 50-100, 1-150, 25-150, 50-150, 100-150, 100-125, 100-150, 150-200, 200-250, 250-500, 500-1000, 1000-1500, 1500-2000, or 2000-2500. In some embodiments, the thermoreversible polymer has a molecular weight which varies, such as from 5 kDa to 750 kDa, such as from 10 kDa to 500 kDa, such as from 15 kDa to 450 kDa, such as from 20 kDa to 400 kDa, such as from 25 kDa to 350 kDa, such as from 30 kDa to 300 kDa, such as from 35 kDa to 250 kDa and including from 40 kDa to 200 kDa. In certain embodiments, the thermoreversible polymer has a molecular weight of from 10 kDa to 500 kDa.

In some embodiments, R¹ is a C1-C6 alkyl. In some instances, R1 is an alkyl group selected from the group consisting of n-butyl, isobutyl, tert-butyl, n-propyl, pentyl, isopropyl and isopentyl. In certain instances, R¹ is n-butyl.

In some embodiments, R² is an alkoxy group. In some instances, R² is a C1-C6 alkoxy group selected from the group consisting of methoxy, ethoxy, n-propoxy, isoproxy, n-butoxy, isobutoxy, tert-butoxy, pentoxy and isopentoxy. In certain instances, R² is methoxy.

In certain instances, a>0.8; 0.2>b>0; 0.1>c>0, and 0.1>d>0.

In some embodiments, the thermoreversible polymer is a polymer of formula (X):

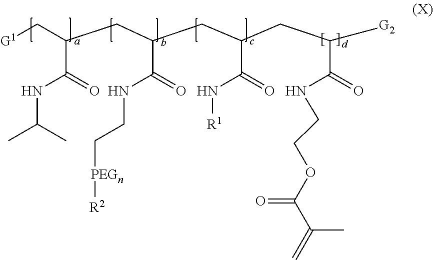

wherein:
a, b, c, and d are molar fractions of the co-monomers, where a, b, c and d are each greater than zero;
PEGₙ is a polyethyleneglycol polymer and n is an integer from 1 to 2500;
R¹ is an alkyl or a substituted alkyl;
R² is alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl;
G¹ and G² are each independently selected from a polymer segment, a terminal group, a linker and a linked modifying agent. In some embodiments, G¹ and G² are each independently a chain-transfer agent. For example, the chain-transfer agent may be a dithioester, dithiocarbamate, trithiocarbonate or a xanthate. In certain embodiments, G¹ is a carboxyl group. In certain instances, G¹ is:

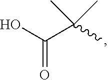

wherein [symbol] is a bond between G¹ and the polymer. In certain embodiments, G² is:

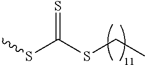

wherein [symbol] is a bond between G² and the polymer.

In some cases, n is an integer from 1-25, 1-50, 1-100, 25-100, 50-100, 1-150, 25-150, 50-150, 100-150, 100-125, 100-150, 150-200, 200-250, 250-500, 500-1000, 1000-1500, 1500-2000, or 2000-2500. In some embodiments, the thermoreversible polymer has a molecular weight which varies, such as from 5 kDa to 750 kDa, such as from 10 kDa to 500 kDa, such as from 15 kDa to 450 kDa, such as from 20 kDa to 400 kDa, such as from 25 kDa to 350 kDa, such as from 30 kDa to 300 kDa, such as from 35 kDa to 250 kDa and including from 40 kDa to 200 kDa. In certain embodiments, the thermoreversible polymer has a molecular weight of from 10 kDa to 500 kDa.

In some embodiments, R¹ is a C1-C6 alkyl. In some instances, R1 is an alkyl group selected from the group consisting of n-butyl, isobutyl, tert-butyl, n-propyl, pentyl, isopropyl and isopentyl. In certain instances, R¹ is n-butyl.

In some embodiments, R² is an alkoxy group. In some instances, R² is a C1-C6 alkoxy group selected from the group consisting of methoxy, ethoxy, n-propoxy, isoproxy, n-butoxy, isobutoxy, tert-butoxy, pentoxy and isopentoxy. In certain instances, R² is methoxy.

In certain instances, a>0.8; 0.2>b>0; 0.1>c>0, and 0.1>d>0.

In some embodiments, the thermoreversible polymer is a polymer of formula (XI):

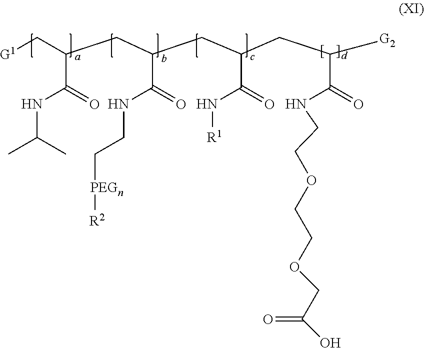

wherein:
a, b, c, and d are molar fractions of the co-monomers, where a, b, c and d are each greater than zero;
PEGₙ is a polyethyleneglycol polymer and n is an integer from 1 to 2500;
R¹ is an alkyl or a substituted alkyl;
R² is alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl;
G¹ and G² are each independently selected from a polymer segment, a terminal group, a linker and a linked modifying agent. In some embodiments, G¹ and G² are each independently a chain-transfer agent. For example, the chain-transfer agent may be a dithioester, dithiocarbamate, trithiocarbonate or a xanthate. In certain embodiments, G¹ is a carboxyl group. In certain instances, G¹ is:

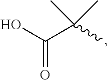

wherein [symbol] is a bond between G¹ and the polymer. In certain embodiments, G² is:

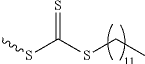

wherein [symbol] is a bond between G² and the polymer.

In some cases, n is an integer from 1-25, 1-50, 1-100, 25-100, 50-100, 1-150, 25-150, 50-150, 100-150, 100-125, 100-150, 150-200, 200-250, 250-500, 500-1000, 1000-1500, 1500-2000, or 2000-2500. In some embodiments, the thermoreversible polymer has a molecular weight which varies, such as from 5 kDa to 750 kDa, such as from 10 kDa to 500 kDa, such as from 15 kDa to 450 kDa, such as from 20 kDa to 400 kDa, such as from 25 kDa to 350 kDa, such as from 30 kDa to 300 kDa, such as from 35 kDa to 250 kDa and including from 40 kDa to 200 kDa. In certain embodiments, the thermoreversible polymer has a molecular weight of from 10 kDa to 500 kDa.

In some embodiments, R¹ is a C1-C6 alkyl. In some instances, R1 is an alkyl group selected from the group consisting of n-butyl, isobutyl, tert-butyl, n-propyl, pentyl, isopropyl and isopentyl. In certain instances, R¹ is n-butyl.

In some embodiments, R² is an alkoxy group. In some instances, R² is a C1-C6 alkoxy group selected from the group consisting of methoxy, ethoxy, n-propoxy, isoproxy, n-butoxy, isobutoxy, tert-butoxy, pentoxy and isopentoxy. In certain instances, R² is methoxy.

In certain instances, a>0.8; 0.2>b>0; 0.1>c>0, and 0.1>d>0.

In some embodiments, the thermoreversible polymer is a polymer of formula (XII):

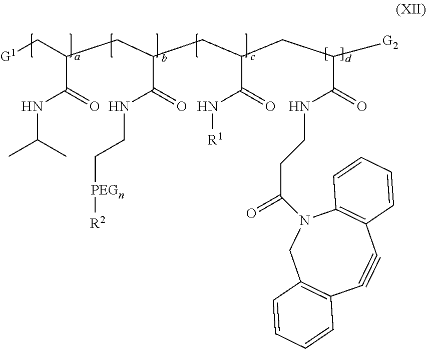

wherein:
a, b, c, and d are molar fractions of the co-monomers, where a, b, c and d are each greater than zero;
PEGₙ is a polyethyleneglycol polymer and n is an integer from 1 to 2500;
R¹ is an alkyl or a substituted alkyl;
R² is alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl;
G¹ and G² are each independently selected from a polymer segment, a terminal group, a linker and a linked modifying agent. In some embodiments, G¹ and G² are each independently a chain-transfer agent. For example, the chain-transfer agent may be a dithioester, dithiocarbamate, trithiocarbonate or a xanthate. In certain embodiments, G¹ is a carboxyl group. In certain instances, G¹ is:

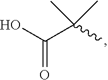

wherein [symbol] is a bond between G¹ and the polymer. In certain embodiments, G² is:

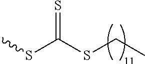

wherein [symbol] is a bond between G² and the polymer.

In some cases, n is an integer from 1-25, 1-50, 1-100, 25-100, 50-100, 1-150, 25-150, 50-150, 100-150, 100-125, 100-150, 150-200, 200-250, 250-500, 500-1000, 1000-1500, 1500-2000, or 2000-2500. In some embodiments, the thermoreversible polymer has a molecular weight which varies, such as from 5 kDa to 750 kDa, such as from 10 kDa to 500 kDa, such as from 15 kDa to 450 kDa, such as from 20 kDa to 400 kDa, such as from 25 kDa to 350 kDa, such as from 30 kDa to 300 kDa, such as from 35 kDa to 250 kDa and including from 40 kDa to 200 kDa. In certain embodiments, the thermoreversible polymer has a molecular weight of from 10 kDa to 500 kDa.

In some embodiments, R¹ is a C1-C6 alkyl. In some instances, R1 is an alkyl group selected from the group consisting of n-butyl, isobutyl, tert-butyl, n-propyl, pentyl, isopropyl and isopentyl. In certain instances, R¹ is n-butyl.

In some embodiments, R² is an alkoxy group. In some instances, R² is a C1-C6 alkoxy group selected from the group consisting of methoxy, ethoxy, n-propoxy, isoproxy, n-butoxy, isobutoxy, tert-butoxy, pentoxy and isopentoxy. In certain instances, R² is methoxy.

In certain instances, a>0.8; 0.2>b>0; 0.1>c>0, and 0.1>d>0.

In some embodiments, the thermoreversible polymer is a polymer of formula (XIII):

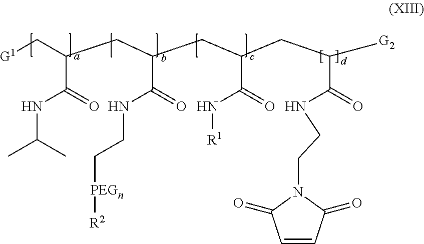

wherein:
a, b, c, and d are molar fractions of the co-monomers, where a, b, c and d are each greater than zero;
PEGₙ is a polyethyleneglycol polymer and n is an integer from 1 to 2500;
R¹ is an alkyl or a substituted alkyl;
R² is alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl;
G¹ and G² are each independently selected from a polymer segment, a terminal group, a linker and a linked modifying agent. In some embodiments, G¹ and G² are each independently a chain-transfer agent. For example, the chain-transfer agent may be a dithioester, dithiocarbamate, trithiocarbonate or a xanthate. In certain embodiments, G¹ is a carboxyl group. In certain instances, G¹ is:

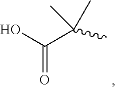

wherein [symbol] is a bond between G¹ and the polymer. In certain embodiments, G² is:

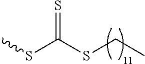

wherein [symbol] is a bond between G² and the polymer.

In some cases, n is an integer from 1-25, 1-50, 1-100, 25-100, 50-100, 1-150, 25-150, 50-150, 100-150, 100-125, 100-150, 150-200, 200-250, 250-500, 500-1000, 1000-1500, 1500-2000, or 2000-2500. In some embodiments, the thermoreversible polymer has a molecular weight which varies, such as from 5 kDa to 750 kDa, such as from 10 kDa to 500 kDa, such as from 15 kDa to 450 kDa, such as from 20 kDa to 400 kDa, such as from 25 kDa to 350 kDa, such as from 30 kDa to 300 kDa, such as from 35 kDa to 250 kDa and including from 40 kDa to 200 kDa. In certain embodiments, the thermoreversible polymer has a molecular weight of from 10 kDa to 500 kDa.

In some embodiments, R¹ is a C1-C6 alkyl. In some instances, R1 is an alkyl group selected from the group consisting of n-butyl, isobutyl, tert-butyl, n-propyl, pentyl, isopropyl and isopentyl. In certain instances, R¹ is n-butyl.

In some embodiments, R² is an alkoxy group. In some instances, R² is a C1-C6 alkoxy group selected from the group consisting of methoxy, ethoxy, n-propoxy, isoproxy, n-butoxy, isobutoxy, tert-butoxy, pentoxy and isopentoxy. In certain instances, R² is methoxy.

In certain instances, a>0.8; 0.2>b>0; 0.1>c>0, and 0.1>d>0.

In some embodiments, the thermoreversible polymer is a polymer of formula (XIV):

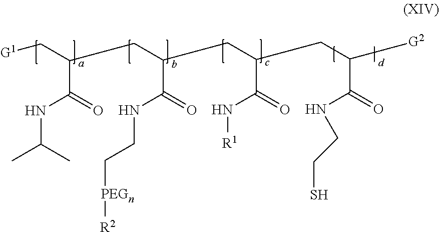

wherein:
a, b, c, and d are molar fractions of the co-monomers, where a, b, c and d are each greater than zero;
PEGₙ is a polyethyleneglycol polymer and n is an integer from 1 to 2500;
R¹ is an alkyl or a substituted alkyl;
R² is alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl;
G¹ and G² are each independently selected from a polymer segment, a terminal group, a linker and a linked modifying agent. In some embodiments, G¹ and G² are each independently a chain-transfer agent. For example, the chain-transfer agent may be a dithioester, dithiocarbamate, trithiocarbonate or a xanthate. In certain embodiments, G¹ is a carboxyl group. In certain instances, G¹ is:

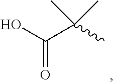

wherein [symbol] is a bond between G¹ and the polymer. In certain embodiments, G² is:

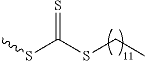

wherein [symbol] is a bond between G² and the polymer.

In some cases, n is an integer from 1-25, 1-50, 1-100, 25-100, 50-100, 1-150, 25-150, 50-150, 100-150, 100-125, 100-150, 150-200, 200-250, 250-500, 500-1000, 1000-1500, 1500-2000, or 2000-2500. In some embodiments, the thermoreversible polymer has a molecular weight which varies, such as from 5 kDa to 750 kDa, such as from 10 kDa to 500 kDa, such as from 15 kDa to 450 kDa, such as from 20 kDa to 400 kDa, such as from 25 kDa to 350 kDa, such as from 30 kDa to 300 kDa, such as from 35 kDa to 250 kDa and including from 40 kDa to 200 kDa. In certain embodiments, the thermoreversible polymer has a molecular weight of from 10 kDa to 500 kDa.

In some embodiments, R¹ is a C1-C6 alkyl. In some instances, R1 is an alkyl group selected from the group consisting of n-butyl, isobutyl, tert-butyl, n-propyl, pentyl, isopropyl and isopentyl. In certain instances, R¹ is n-butyl.

In some embodiments, R² is an alkoxy group. In some instances, R² is a C1-C6 alkoxy group selected from the group consisting of methoxy, ethoxy, n-propoxy, isoproxy, n-butoxy, isobutoxy, tert-butoxy, pentoxy and isopentoxy. In certain instances, R² is methoxy.

In certain instances, a>0.8; 0.2>b>0; 0.1>c>0, and 0.1>d>0.

In some embodiments, the thermoreversible polymer is a polymer of formula (XV):

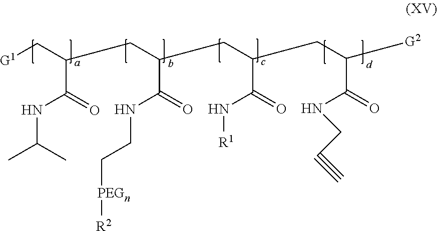

wherein:
a, b, c, and d are molar fractions of the co-monomers, where a, b, c and d are each greater than zero;
PEGₙ is a polyethyleneglycol polymer and n is an integer from 1 to 2500;
R¹ is an alkyl or a substituted alkyl;
R² is alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl;
G¹ and G² are each independently selected from a polymer segment, a terminal group, a linker and a linked modifying agent. In some embodiments, G¹ and G² are each independently a chain-transfer agent. For example, the chain-transfer agent may be a dithioester, dithiocarbamate, trithiocarbonate or a xanthate. In certain embodiments, G¹ is a carboxyl group. In certain instances, G¹ is:

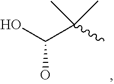

wherein [symbol] is a bond between G¹ and the polymer. In certain embodiments, G² is:

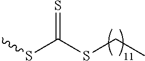

wherein [symbol] is a bond between G² and the polymer.

In some cases, n is an integer from 1-25, 1-50, 1-100, 25-100, 50-100, 1-150, 25-150, 50-150, 100-150, 100-125, 100-150, 150-200, 200-250, 250-500, 500-1000, 1000-1500, 1500-2000, or 2000-2500. In some embodiments, the thermoreversible polymer has a molecular weight which varies, such as from 5 kDa to 750 kDa, such as from 10 kDa to 500 kDa, such as from 15 kDa to 450 kDa, such as from 20 kDa to 400 kDa, such as from 25 kDa to 350 kDa, such as from 30 kDa to 300 kDa, such as from 35 kDa to 250 kDa and including from 40 kDa to 200 kDa. In certain embodiments, the thermoreversible polymer has a molecular weight of from 10 kDa to 500 kDa.

In some embodiments, R¹ is a C1-C6 alkyl. In some instances, R1 is an alkyl group selected from the group consisting of n-butyl, isobutyl, tert-butyl, n-propyl, pentyl, isopropyl and isopentyl. In certain instances, R¹ is n-butyl.

In some embodiments, R² is an alkoxy group. In some instances, R² is a C1-C6 alkoxy group selected from the group consisting of methoxy, ethoxy, n-propoxy, isoproxy, n-butoxy, isobutoxy, tert-butoxy, pentoxy and isopentoxy. In certain instances, R² is methoxy.

In certain instances, a>0.8; 0.2>b>0; 0.1>c>0, and 0.1>d>0.

### Methods of Making a Thermoreversible Polymer

The present disclosure provides a method of making a thermoreversible polymer of the present disclosure. In some cases, methods include: co-polymerizing N-isopropylacrylamide and N-acryloxysuccinimide to generate a first copolymer comprising an acrylamide backbone; contacting the copolymer with an alkyl amine and an alkoxy-polyethylene glycol amine to generate a second copolymer; and contacting the second copolymer with isopropylamine to generate a polymer of formula I:

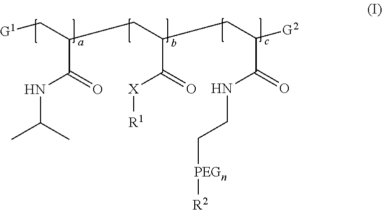

wherein:
a, b and c are molar fractions of the co-monomers, where a, b and c are each greater than zero;
PEGₙ is a polyethyleneglycol polymer and n is an integer from 1 to 2500.
X is independently selected from C, O, and NH. In some instances, X is O. In some instances, X is NH.
R¹ is an alkyl or a substituted alkyl;
R² is alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl; and
G¹ and G² are each independently selected from a polymer segment, a terminal group, a linker and a linked modifying agent. In some embodiments, G¹ and G² are each independently a chain-transfer agent. For example, the chain-transfer agent may be a dithioester, dithiocarbamate, trithiocarbonate or a xanthate.

In some cases, n is an integer from 1-25, 1-50, 1-100, 25-100, 50-100, 1-150, 25-150, 50-150, 100-150, 100-125, 100-150, 150-200, 200-250, 250-500, 500-1000, 1000-1500, 1500-2000, or 2000-2500. In some embodiments, the thermoreversible polymer has a molecular weight which varies, such as from 5 kDa to 750 kDa, such as from 10 kDa to 500 kDa, such as from 15 kDa to 450 kDa, such as from 20 kDa to 400 kDa, such as from 25 kDa to 350 kDa, such as from 30 kDa to 300 kDa, such as from 35 kDa to 250 kDa and including from 40 kDa to 200 kDa. In certain embodiments, the thermoreversible polymer has a molecular weight of from 10 kDa to 500 kDa.

In some embodiments, R¹ is a C1-C6 alkyl. In some instances, R1 is an alkyl group selected from the group consisting of n-butyl, isobutyl, tert-butyl, n-propyl, pentyl, isopropyl and isopentyl. In certain instances, R¹ is n-butyl.

In some embodiments, R² is an alkoxy group. In some instances, R² is a C1-C6 alkoxy group selected from the group consisting of methoxy, ethoxy, n-propoxy, isoproxy, n-butoxy, isobutoxy, tert-butoxy, pentoxy and isopentoxy. In certain instances, R² is methoxy.

In certain instances, a>0.8; 0.2>b>0; and 0.1>c>0.

In some cases, methods include: co-polymerizing N-isopropylacrylamide, N-acryloxysuccinimide and an alkyl methacrylate to generate a first copolymer comprising an acrylamide backbone; contacting the copolymer with an alkoxy-polyethylene glycol amine to generate a second copolymer; and contacting the second copolymer with isopropylamine to generate a polymer of formula I:

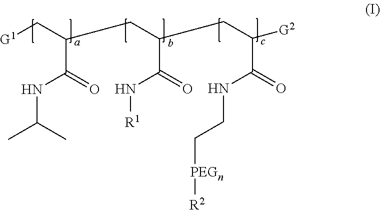

wherein:
a, b and c are molar fractions of the co-monomers, where a, b and c are each greater than zero;
PEGₙ is a polyethyleneglycol polymer and n is an integer from 1 to 2500.
R¹ is an alkyl or a substituted alkyl;
R² is alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl; and
G¹ and G² are each independently selected from a polymer segment, a terminal group, a linker and a linked modifying agent. In some embodiments, G¹ and G² are each independently a chain-transfer agent. For example, the chain-transfer agent may be a dithioester, dithiocarbamate, trithiocarbonate or a xanthate.

In some cases, n is an integer from 1-25, 1-50, 1-100, 25-100, 50-100, 1-150, 25-150, 50-150, 100-150, 100-125, 100-150, 150-200, 200-250, 250-500, 500-1000, 1000-1500, 1500-2000, or 2000-2500. In some embodiments, the thermoreversible polymer has a molecular weight which varies, such as from 5 kDa to 750 kDa, such as from 10 kDa to 500 kDa, such as from 15 kDa to 450 kDa, such as from 20 kDa to 400 kDa, such as from 25 kDa to 350 kDa, such as from 30 kDa to 300 kDa, such as from 35 kDa to 250 kDa and including from 40 kDa to 200 kDa. In certain embodiments, the thermoreversible polymer has a molecular weight of from 10 kDa to 500 kDa.

In some cases, the alkyl methacrylate is butyl methacrylate.

In some embodiments, R¹ is a C1-C6 alkyl. In some instances, R1 is an alkyl group selected from the group consisting of n-butyl, isobutyl, tert-butyl, n-propyl, pentyl, isopropyl and isopentyl. In certain instances, R¹ is n-butyl.

In some embodiments, R² is an alkoxy group. In some instances, R² is a C1-C6 alkoxy group selected from the group consisting of methoxy, ethoxy, n-propoxy, isoproxy, n-butoxy, isobutoxy, tert-butoxy, pentoxy and isopentoxy. In certain instances, R² is methoxy.

In certain instances, a>0.8; 0.2>b>0; and 0.1>c>0.

In certain embodiments, methods include co-polymerizing N-isopropylacrylamide and N-acryloxysuccinimide to generate a first copolymer comprising an acrylic backbone; contacting the copolymer with butylamine and methoxy-polyethylene glycol amine to generate a second copolymer; and contacting the second copolymer with isopropylamine to generate a polymer of formula II:

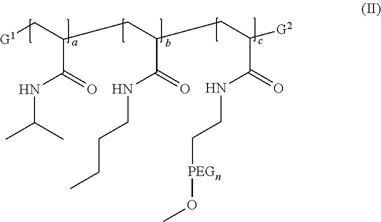

wherein n is 1 to 25; and
G¹ and G² are each independently selected from a polymer segment, a terminal group, a linker and a linked modifying agent. In some embodiments, G¹ and G² are each independently a chain-transfer agent. For example, the chain-transfer agent may be a dithioester, dithiocarbamate, trithiocarbonate or a xanthate.

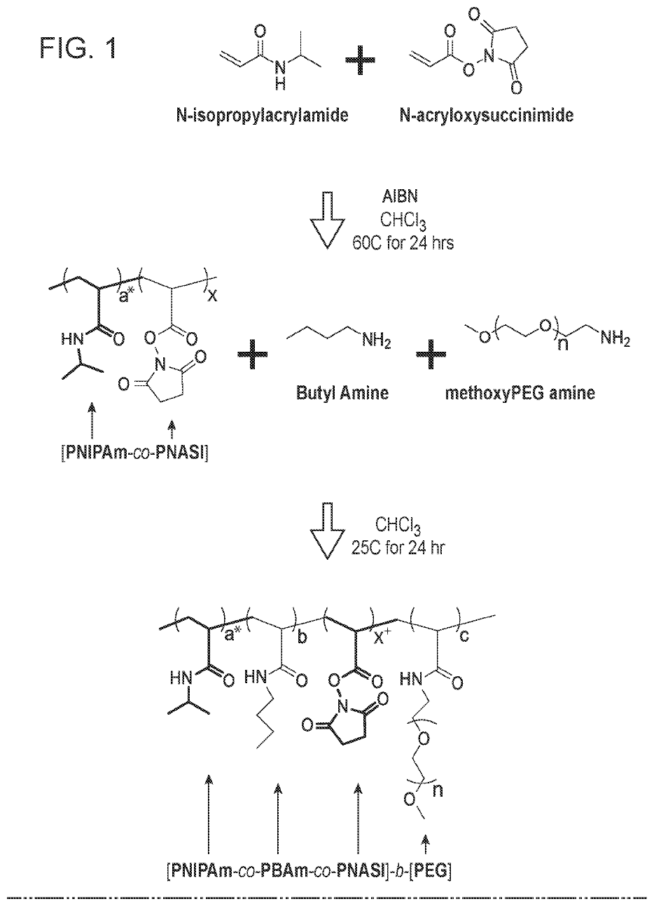

In some cases, the reaction, as depicted in FIG. 1, of the N-isopropylacrylamide with the N-acryloxysuccinimide includes from about 50 to about 95 mol % N-isopropylacrylamide and the remaining mol % (to 100 mol %) N-acryloxysuccinimide. For example, in some cases, the reaction, as depicted in FIG. 1, of the N-isopropylacrylamide with the N-acryloxysuccinimide includes: i) from about 50 mol % to about 70 mol % N-isopropylacrylamide; and ii) from about 50 mol % to about 30 mol % N-acryloxysuccinimide. As another example, in some cases, the reaction, as depicted in FIG. 1, of the N-isopropylacrylamide with the N-acryloxysuccinimide includes: i) from about 70 mol % to about 95 mol % N-isopropylacrylamide; and ii) from about 30 mol % to about 5 mol % N-acryloxysuccinimide. As another example, in some cases, the reaction, as depicted in FIG. 1, of the N-isopropylacrylamide with the N-acryloxysuccinimide includes: i) from about 80 mol % to about 90 mol % N-isopropylacrylamide; and ii) from about 20 mol % to about 10 mol % N-acryloxysuccinimide. In some cases, the reaction, as depicted in FIG. 1, of the N-isopropylacrylamide with the N-acryloxysuccinimide includes: i) 70, 75, 80, 85, 90, or 95 mol % N-isopropylacrylamide; and ii) from about 30, 25, 20, 15, 10, or 5 mol % N-acryloxysuccinimide. This reaction can be conducted at a temperature of from 40° C. to about 80° C. (e.g., from about 40° C. to about 45° C., from about 45° C. to about 50° C., from about 50° C. to about 55° C., from about 55° C. to about 60° C., from about 60° C. to about 65° C., from about 65° C. to about 70° C., or from about 70° C. to about 80° C. The reaction can be carried out for a period of time of from about 18 hours to about 36 hours (e.g., from about 18 hours to about 24 hours, from about 24 hours to about 30 hours, or from about 30 hours to about 36 hours. Such a reaction generates the intermediate referred to in FIG. 1 as PNIPAm-co-PNASI.

In some cases, the reaction, as depicted in FIG. 1, of the PNIPAm-co-PNASI intermediate with butyl amine and methoxyPEG amine includes from about 5 mol % to about 30 mol % (e.g., about 5 mol %, about 10 mol %, about 15 mol %, about 20 mol %, about 25 mol %, or about 30 mol %) butyl amine and methoxyPEG amine at about from 20 wt % to about 50 wt % of PNIPAAm (poly(N-isopropylacrylamide) (e.g., from about 20 wt % to about 25 wt %, from about 25 wt % to about 30 wt %, from about 30 wt % to about 35 wt %, from about 35 wt % to about 40 wt %, from about 40 wt % to about 45 wt %, or from about 45 wt % to about 50 wt % to PNIPAAm.

In some cases, methods include: co-polymerizing N-isopropylacrylamide and N-acryloxysuccinimide to generate a first copolymer comprising an acrylamide backbone; contacting the copolymer with an alkyl amine (e.g., butyl amine) and an alkoxy-polyethylene glycol amine to generate a second copolymer; contacting the second copolymer with an aminoalkyl methacrylate (e.g., 2-amino methacrylate) to generate a third copolymer and contacting the third copolymer with isopropylamine to generate a polymer of formula IV:

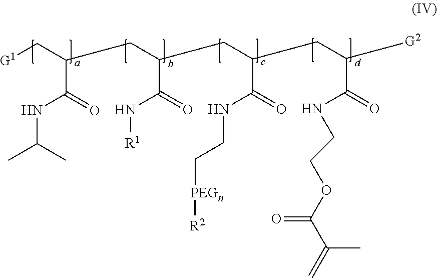

wherein:
a, b, c, and d are molar fractions of the co-monomers, where a, b, c and d are each greater than zero;
PEGₙ is a polyethyleneglycol polymer and n is an integer from 1 to 2500;
R¹ is an alkyl or a substituted alkyl;
R² is alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl;
G¹ and G² are each independently selected from a polymer segment, a terminal group, a linker and a linked modifying agent. In some embodiments, G¹ and G² are each independently a chain-transfer agent. For example, the chain-transfer agent may be a dithioester, dithiocarbamate, trithiocarbonate or a xanthate. In certain embodiments, G¹ is a carboxyl group. In certain instances, G¹ is:

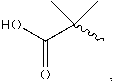

wherein [symbol] is a bond between G¹ and the polymer. In certain embodiments, G² is:

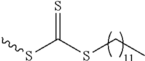

wherein [symbol] is a bond between G² and the polymer.

In some cases, n is an integer from 1-25, 1-50, 1-100, 25-100, 50-100, 1-150, 25-150, 50-150, 100-150, 100-125, 100-150, 150-200, 200-250, 250-500, 500-1000, 1000-1500, 1500-2000, or 2000-2500. In some embodiments, the thermoreversible polymer has a molecular weight which varies, such as from 5 kDa to 750 kDa, such as from 10 kDa to 500 kDa, such as from 15 kDa to 450 kDa, such as from 20 kDa to 400 kDa, such as from 25 kDa to 350 kDa, such as from 30 kDa to 300 kDa, such as from 35 kDa to 250 kDa and including from 40 kDa to 200 kDa. In certain embodiments, the thermoreversible polymer has a molecular weight of from 10 kDa to 500 kDa.

In some embodiments, R¹ is a C1-C6 alkyl. In some instances, R1 is an alkyl group selected from the group consisting of n-butyl, isobutyl, tert-butyl, n-propyl, pentyl, isopropyl and isopentyl. In certain instances, R¹ is n-butyl.

In some cases, methods include a reversible addition-fragmentation chain-transfer (RAFT) polymerization comprising the steps of: co-polymerizing N-isopropylacrylamide and N-acryloxysuccinimide with a RAFT agent (e.g., a DMP RAFT agent) to generate a first copolymer comprising an acrylamide backbone; contacting the copolymer with an alkyl amine and an alkoxy-polyethylene glycol amine to generate a second copolymer; and contacting the second copolymer with isopropylamine to generate a polymer of formula I:

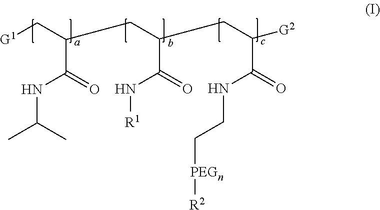

wherein:
a, b, and c are molar fractions of the co-monomers, where a, b, and c are each greater than zero;
PEGₙ is a polyethyleneglycol polymer and n is an integer from 1 to 2500;
R¹ is an alkyl or a substituted alkyl;
R² is alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl; and
In certain embodiments, G¹ is a carboxyl group. In certain instances, G¹ is:

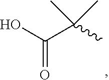

wherein [symbol] is a bond between G¹ and the polymer. In certain embodiments, G² is:

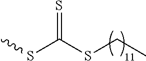

wherein [symbol] is a bond between G² and the polymer.

In some cases, n is an integer from 1-25, 1-50, 1-100, 25-100, 50-100, 1-150, 25-150, 50-150, 100-150, 100-125, 100-150, 150-200, 200-250, 250-500, 500-1000, 1000-1500, 1500-2000, or 2000-2500. In some embodiments, the thermoreversible polymer has a molecular weight which varies, such as from 5 kDa to 750 kDa, such as from 10 kDa to 500 kDa, such as from 15 kDa to 450 kDa, such as from 20 kDa to 400 kDa, such as from 25 kDa to 350 kDa, such as from 30 kDa to 300 kDa, such as from 35 kDa to 250 kDa and including from 40 kDa to 200 kDa. In certain embodiments, the thermoreversible polymer has a molecular weight of from 10 kDa to 500 kDa.

In some embodiments, R¹ is a C1-C6 alkyl. In some instances, R1 is an alkyl group selected from the group consisting of n-butyl, isobutyl, tert-butyl, n-propyl, pentyl, isopropyl and isopentyl. In certain instances, R¹ is n-butyl.

In some embodiments, R² is an alkoxy group. In some instances, R² is a C1-C6 alkoxy group selected from the group consisting of methoxy, ethoxy, n-propoxy, isoproxy, n-butoxy, isobutoxy, tert-butoxy, pentoxy and isopentoxy. In certain instances, R² is methoxy.

In certain instances, a>0.8; 0.2>b>0; and 0.1>c>0.

### Compositions

The present disclosure provides a composition including two or more thermoreversible polymers of the present disclosure. In some cases, the composition includes a mixture of a low MW thermoreversible polymer (e.g., having a MW of 100 kDa or less, such as 75 kDa or less, or 50 kDa or less) and a high MW thermoreversible polymer (e.g., having a MW of 100 kDa or more, such as 200 kDa or more, 300 kDa or more, 500 kDa or more, or even more).

Aspects of the present disclosure include a hydrogel composition including: a) a thermoreversible polymer of the present disclosure; and b) an aqueous solution, e.g., a buffered aqueous solution. When the hydrogel composition is below its sol-gel transition temperature, the composition can be a homogeneous solution, such that any cells that are present in the solution may be easily removed (e.g., by centrifugation). When the hydrogel composition is above its sol-gel transition temperature, the thermoreversible polymer provides a three-dimensional matrix that finds use in the incubation, growth and/or differentiation of cells of interest.

Any convenient buffered aqueous solutions that find use in the incubation and/or differentiation of cells of interest may be utilized in the subject hydrogel compositions. The buffered aqueous solution may include any convenient components of interest.

In some instances, the hydrogel composition further includes cells of interest (e.g., as described herein). In certain embodiments, the hydrogel composition includes stem cells selected from the group consisting of (a) adult stem cell derived from bone marrow, umbilical tissues, or placenta; (b) neural stem cell; and (c) embryonic stem cell. In some cases, the cells are immune cells, e.g., T cells, natural killer cells, and the like. In some cases, the cells are genetically modified with one or more nucleic acids. For example, T cells can be genetically modified with a nucleic acid comprising a nucleotide sequence encoding a chimeric antigen receptor.

In certain instances, the thermoreversible polymer is a solid, semi-solid, or gel at 20° C. or more, such as 21° C. or more, 22° C. or more, 23° C. or more, 24° C. or more, 25° C. or more, 26° C. or more, 27° C. or more, 28° C. or more, 29° C. or more, 30° C. or more, 31° C. or more, 32° C. or more, 33° C. or more, 34° C. or more, 35° C. or more, 36° C. or more, or even more. In certain embodiments, the thermoreversible polymer is a solid at 37° C.

### Thermoreversible Polymer-Cell Compositions

The present disclosure provides a composition comprising: a) a thermoreversible polymer of the present disclosure; and b) cells embedded or suspended within the polymer. A thermoreversible polymer-cell composition of the present disclosure is useful for generating a desired number of cells, by culturing the thermoreversible polymer-cell composition under conditions and for a period of time sufficient to generate the desired number of cells. Such cells can include stem cells, differentiated cells, and the like. A thermoreversible polymer-cell composition of the present disclosure is useful for differentiating cells, e.g., to generate a desired number of differentiated cells. A thermoreversible polymer-cell composition of the present disclosure can be implanted into an individual in need thereof, where cells proliferate and/or differentiated within the implanted thermoreversible polymer-cell composition, and migrate out of the implanted thermoreversible polymer-cell composition.

### Methods for Culturing Cells

A thermoreversible polymer of the present disclosure can be used to culture cells in vitro or in vivo. Thus, the present disclosure provides methods of culturing cells, the methods involving contacting the cells with the thermoreversible polymer; and culturing the cell-containing thermoreversible polymer under conditions suitable for growth and/or differentiation of the cells. In some cases, a method of the present disclosure comprises culturing cells contained within (e.g., embedded in; suspended in; etc.) a hydrogel composition of the present disclosure.

In some cases, a method of the present disclosure for culturing cells comprises culturing the cells in a hydrogel composition of the present disclosure at a temperature (e.g., from about 30° C. to about 37° C.; e.g., at 37° C.) at which the hydrogel composition is a semi-solid (e.g., a gel). In some cases, a method of the present disclosure for culturing cells comprises culturing the cells in a hydrogel composition of the present disclosure at a temperature (e.g., from about 4° C. to about 10° C.; e.g., at 4° C.) at which the hydrogel composition is a liquid.

A method of the present disclosure for culturing cells can be used to generate a desired number of cells, including differentiated cells and stem cells. For example, a method of the present disclosure can be used to generate from 10² cells to about 10⁹ cells, e.g., from about 10² cells to about 5×10² cells, from about 5×10² cells to about 10³ cells, from about 10³ cells to about 5×10³ cells, from about 5×10³ cells to about 10⁴ cells, from about 10⁴ cells to about 5×10⁴ cells, from about 5×10⁴ cells to about 10⁵ cells, from about 10⁵ cells to about 5×10⁵ cells, from about 5×10⁵ cells to about 10⁶ cells, from about 10⁶ cells to about 5×10⁶ cells, from about 5×10⁶ cells to about 10⁷ cells, from about 10⁷ cells to about 5×10⁷ cells, from about 5×10⁷ cells to about 10⁸ cells, from about 10⁸ cells to about 5×10⁸ cells, or from about 5×10⁸ cells to about 10⁹ cells. In some cases, a method of the present disclosure can be used to generate more than 10⁹ cells, e.g., from 10⁹ cells to 5×10⁹ cells, from 5×10⁹ cells to 10¹⁰ cells, from 10¹⁰ cells to 5×10¹⁰ cells, from 5×10¹⁰ cells to 10¹¹ cells, from 10¹¹ cells to 5×10¹¹ cells, from 5×10¹¹ cells to 10¹² cells, from 10¹² cells to 5×10¹² cells, from 5×10¹² cells to 10¹³ cells, from 10¹³ cells to 5×10¹³ cells, from 5×10¹³ cells to 10¹⁴ cells, from 10¹⁴ cells to 5×10¹⁴ cells, or from 5×10¹⁴ cells to 10¹⁵ cells.

Cells can be cultured in a hydrogel composition of the present disclosure can be present in the hydrogel composition (e.g., embedded within the hydrogel composition; suspended in the hydrogel composition; etc.) at a density of from 10 cells per mL (or cubic centimeters) hydrogel to about 10⁸ cells per mL, e.g., from about 10 cells per mL to about 10² cells per mL, from about 10² cells per mL to about 10⁴ cells per mL, from about 10⁴ cells per mL to about 10⁶ cells per mL, or from about 10⁶ cells per mL to about 10⁸ cells per mL.

In some cases, the hydrogel composition maintains pluripotency of pluripotent stem cells contained within the hydrogel composition. For example, in some cases, the hydrogel composition maintains pluripotency of pluripotent stem cells contained within the hydrogel composition when cultured in the hydrogel composition for a period of time of 1 day to 6 months or more. For example, in some cases, the hydrogel composition maintains pluripotency of pluripotent stem cells contained within the hydrogel composition when cultured in the hydrogel composition for a period of time of 1 day to 7 days, from 1 week to 2 weeks, from 2 weeks to one month, from one month to 2 months, from 2 months to 4 months, or from 4 months to 6 months. For example, in some cases, the hydrogel composition maintains pluripotency of at least 10%, at least 20%, at least 30%, at least 40%, at least 50%, at least 60%, at least 70%, at least 80%, at least 90%, or more than 90%, of the pluripotent stem cells contained within the hydrogel composition when cultured in the hydrogel composition for a period of time of 1 day to 7 days, from 1 week to 2 weeks, from 2 weeks to one month, from one month to 2 months, from 2 months to 4 months, or from 4 months to 6 months. In some cases, the hydrogel composition provides sufficient time for cell propagation. In some cases, the cells cultured in the hydrogel composition maintain pluripotency after 1 passage, after 2 passages, after 3 passages or after more than 3 passages. In some cases, the hydrogel composition maintains pluripotency of human pluripotent stem cells (hPSCs). In some cases, the hPSCs are H1 embryonic stem cells (H1ESCs). In some cases, the hPSCs are H9 embryonic stem cells (H9ESCs). In some cases, the hydrogel composition maintains pluripotency of induced pluripotent stem cells (iPSCs). In some cases, the iPSCs cultured in the hydrogel maintain pluripotency after 1 passage, after 2 passages, after 3 passages, or after more than 3 passages (e.g., after 4 passages, after 5 passages, after from 5 to 10 passages, after from 10 to 15 passages, after from 15 to 20 passages, etc.).

In some cases, cells cultured in the hydrogel composition aggregate. For example, in some cases, the cells cultured in the hydrogel composition grow as small aggregates after 1 day in culture. In some cases, cells cultured in the hydrogel composition grow as single cells at 1 day in culture. In some cases, the cells cultured in the hydrogel composition aggregate after 2 days in culture. In some cases, cells cultured in the hydrogel composition aggregate after 3 days in culture. In some cases, cells cultured in the hydrogel composition aggregate after 4 days in culture. In some instances, the cells are H9ESCs. In some cases, H9ESCs grow as small aggregates at 1 day in culture. In some instances, H9ESCs grow as large aggregates at 4 days in culture.

The hydrogel composition can include one or more factors (e.g., polypeptides; small molecules; etc.) that promote proliferation or differentiation of cells cultured in the hydrogel composition. Suitable factors include, e.g., retinoic acid, a Wnt agonist, an Shh signaling pathway agonist, a bone morphogenic protein (BMP) inhibitor (e.g., Noggin), a receptor tyrosine kinase ligand (e.g., epidermal growth factor), nicotinamide, a p38 inhibitor, a dual-Smad inhibitor, a Rock inhibitor, gastrin, an activator of the prostaglanding signalling pathway, fibroblast growth factor (FGF) (e.g., FGF10), a TGF-β inhibitor, Rspondin, an Rspondin mimic, and combinations of two or more of the aforementioned factors. Such factors can be present in the hydrogel composition at concentrations ranging from 1 nM to 100 mM, e.g., from 1 nM to 50 nM, from 50 nM to 100 nM, from 100 nM to 0.5 μM, from 0.5 μM to 1 μM, from 1 μM to 50 M, from 50 μM to 100 μM, from 100 μM to 0.5 mM, from 0.5 μM to 1 mM, from 1 mM to 50 mM, or from 50 mM to 100 mM. Such factors can be present in the hydrogel composition at concentrations ranging from 1 ng/ml to 1 mg/ml, e.g., from 1 ng/ml to 50 ng/ml, from 50 ng/ml to 100 ng/ml, from 100 ng/ml to 0.5 μg/ml, from 0.5 μg/ml to 1 μg/ml, from 1 μg/ml to 50 g/ml, from 50 μg/ml to 100 μg/ml, from 100 μg/ml to 500 μg/ml, from 500 μg/ml to 0.1 mg/ml, from 0.1 mg/ml to 0.5 mg/ml, or from 0.5 mg/ml to 1 mg/ml, or more than 1 mg/ml.

In some cases, a hydrogel composition of the present disclosure includes one or more of: Rspondin 1-4 and/or an Rspondin mimic, a BMP inhibitor (for example, Noggin), a TGF-beta inhibitor, a receptor tyrosine kinase ligand (for example, EGF), Nicotinamide, a Wnt agonist (for example, Wnt(3a)), a Wnt antagonist (e.g., IWP-2, IWP-3, IWP-4, Dkk1, and the like), a p38 inhibitor, gastrin, FGF10, HGF and a ROCK inhibitor.

IWP2 has the following structure:

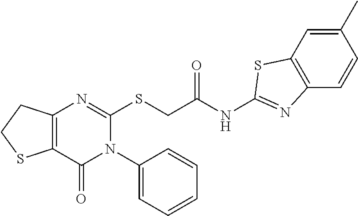 [SMILES: Cc1ccc2nc(NC(=O)CSc3nc4c(c(=O)n3-c3ccccc3)SCC4)sc2c1]

Several classes of natural BMP-binding proteins are known, including Noggin, Chordin and chordin-like proteins comprising chordin domains, Follistatin and follistatin-related proteins comprising a follistatin domain, DAN and DAN-like proteins comprising a DAN cysteine-knot domain, sclerostin/SOST, and apha-2 macroglobulin. A BMP inhibitor is an agent that binds to a BMP molecule to form a complex wherein the BMP activity is reduced, for example by preventing or inhibiting the binding of the BMP molecule to a BMP receptor. Alternatively, the inhibitor may be an agent that binds to a BMP receptor and prevents binding of a BMP ligand to the receptor, for example, an antibody that binds the receptor. A BMP inhibitor may be a protein or small molecule and may be naturally occurring, modified, and/or partially or entirely synthetic. A BMP inhibitor can be Noggin, DAN, or DAN-like proteins including Cerberus and Gremlin. In some cases, the BMP inhibitor is Noggin. The BMP inhibitor (e.g., Noggin) may be used at any suitable concentration. A hydrogel composition of the present disclosure can include Noggin in a concentration of between about 10 ng/ml and about 100 ng/ml of Noggin.

A hydrogel composition of the present disclosure can include one or more Wnt agonists. The Wnt signalling pathway is defined by a series of events that occur when a Wnt protein binds to a cell-surface receptor of a Frizzled receptor family member. This results in the activation of Dishevelled family proteins which inhibit a complex of proteins that includes axin, GSK-3, and the protein APC to degrade intracellular beta-catenin. The resulting enriched nuclear beta-catenin enhances transcription by TCF/LEF family transcription factors. A Wnt agonist is defined as an agent that activates TCF/LEF-mediated transcription in a cell. Wnt agonists can be Wnt agonists that bind and activate a Frizzled receptor family member including any and all of the Wnt family proteins, an inhibitor of intracellular beta-catenin degradation, and activators of TCF/LEF.

Suitable Wnt agonists include a secreted glycoprotein including Wnt-1/Int-1, Wnt-2/Irp (InM-related Protein), Wnt-2b/13, Wnt-3/Int-4, Wnt-3a, Wnt-4, Wnt-5a, Wnt-5b, Wnt-6, Writ-7a, Wnt-7b, Wnt-8a/8d, Wnt-8b, Wnt-9a/14, Wnt-9b/14b/15, Wnt-10a, Wnt-10b/12, WnM 1, and Wnt-16. Other suitable Wnt agonists include the R-spondin family of secreted proteins, which is implicated in the activation and regulation of Wnt signaling pathway and which is comprised of 4 members (R-spondin 1, R-spondin 2, R-spondin 3, and R-spondin-4), and Norrin (also called Nome Disease Protein or NDP), which is a secreted regulatory protein that functions like a Wnt protein in that it binds with high affinity to the Frizzled-4 receptor and induces activation of the Wnt signaling pathway. Also suitable is an R-spondin mimic, for example an agonist of Lgr5 such as an anti-Lgr5 antibody.

Suitable Wnt agonists include a glycogen synthase kinase-3 (GSK-3) inhibitor. Known GSK-3 inhibitors comprise small-interfering RNAs (siRNA), lithium, kenpaullone, 6-Bromoindirubin-30-acetoxime, SB 216763 and SB 415286, and FRAT-family members and FRAT-derived peptides that prevent interaction of GSK-3 with axin.

Suitable Wnt agonists include Wnt-3a, a GSK-3 inhibitor (such as CHIR99021), Wnt 5, Wnt-6a, Norrin, and any other Wnt family protein.

A Wnt agonist can be included in the hydrogel composition in a suitable concentration. For example, CHIR99021 (6-[[2-[[4-(2,4-Dichlorophenyl)-5-(5-methyl-1H-imidazol-2-yl)-2-pyrimidinyl]amino]ethyl]amino]-3-pyridinecarbonitrile) can be included in a final concentration of between 50 nM and 100 μM, for example between 100 nM and 50 μM, between 1 μM and 10 M, between 1 μM and 5 μM, or 3 μM.

Exemplary GSK-3 inhibitors include CHIR 99021 (6-[[2-[[4-(2,4-Dichlorophenyl)-5-(5-methyl-1H-imidazol-2-yl)-2-pyrimidinyl]amino]ethyl]amino]-3-pyridinecarbonitrile; CAS No.: 252917-06-9), SB-216763 (3-(2,4-Dichlorophenyl)-4-(1-methyl-1H-indo-3-yl)-1H-pyrrole-2,5-dione; CAS No.: 280744-09-4), 6-bromoindirubin-3′-oxime (CAS No.: CAS 667463-62-9), Tideglusib (4-Benzyl-2-(naphthalen-1-yl)-1,2,4-thiadiazolidine-3,5-dione), GSK-3 inhibitor 1 (CAS No.: 603272-51-1), AZD1080 (CAS No.: 612487-72-6), TDZD-8 (4-Benzyl-2-methyl-1,2,4-thiadiazolidine-3,5-dione; CAS No.: 327036-89-5), TWS119 (3-[[6-(3-aminophenyl)-7H-pyrrolo[2,3-d]pyrimidin-4-yl]oxy]-phenol; CAS No.: 601514-19-6), CHIR-99021 (CAS No.: 252917-06-9), CHIR-98014 (N6-[2-[[4-(2,4-dichlorophenyl)-5-(1H-imidazol-1-yl)-2-pyrimidinyl]amino]-ethyl]-3-nitro-2,6-Pyridinediamine; CAS No.: 252935-94-7), SB 415286 (3-[(3-Chloro-4-hydroxyphenyl)-amino]-4-(2-nitrophenyl)-1H-pyrrol-2,5-dione; CAS No.: 264218-23-7), LY2090314 (3-(9-fluoro-2-(piperidine-1-carbonyl)-1,2,3,4-tetrahydro-[1,4]diazepino[-6,7,1-hi]indol-7-yl)-4-(imidazo[1,2-a]pyridin-3-yl)-1H-pyrrole-2,5-dione, CAS No.: 603288-22-8), AR-A014418 (N-(4-Methoxybenzyl)-N′-(5-nitro-1,3-thiazol-2-yl)urea; CAS No.: 487021-52-3 and/or IM-12 (3-(4-Fluorophenylethylamino)-1-methyl-4-(2-methyl-1H-indol-3-yl)-1H-pyrr-ole-2,5-dione; CAS No.: 1129669-05-1). Thus, the GSK-3 inhibitor can also be CHIR 99021

A hydrogel composition of the present disclosure can comprise one or more receptor tyrosine kinase ligands. An example of a suitable receptor tyrosine kinase ligand is EGF, which is the ligand for the receptor tyrosine kinase EGFR. Many receptor tyrosine kinase ligands are also mitogenic growth factors.

A hydrogel composition of the present disclosure can include a TGF-β inhibitor. Examples of suitable TGF-β inhibitors include, e.g., 3-(6-methyl-2-pyridinyl)-N-phenyl-4-(4-quinolinyl)-1H-pyrazole-1-carbothioamide (A83-01); 4-[4-(1,3-benzodioxol-5-yl)-5-(2-pyridinyl)-1H-imidazol-2-yl]benzamide (SB-431542); and the like. Suitable TGF-β inhibitors include those listed in Table 1 of U.S. Patent Publication No. 2014/0243227; for example, A83-01, SB-431542, SB-505124, SB-525334, SD-208, LY-36494 and SJN-2511.

A hydrogel composition of the present disclosure can comprise one or more mitogenic growth factor. The one or more mitogenic growth factor may be selected from a family of growth factors comprising epidermal growth factor (EGF), Transforming Growth Factor-alpha (TGF-alpha), basic Fibroblast Growth Factor (bFGF), brain-derived neurotrophic factor (BDNF), and Keratinocyte Growth Factor (KGF).

A hydrogel composition of the present disclosure can include a Rock (Rho-kinase) inhibitor. Suitable Rock inhibitors include, e.g., R-(+)-trans-4-(1-aminoethyl)-N-(4-Pyridyl)cyclohexanecarboxamide dihydrochloride monohydrate (Y-27632, Sigma-Aldrich), 5-(1,4-diazepan-1-ylsulfonyl)isoquinoline (fasudil or HA1077, Cayman Chemical), and (S)-(+)-2-methyl-1-[(4-methyl-5-isoquinolinyl)sulfonyl]-hexahydro-1H-1,4-diazepine dihydrochloride (H-1 152, Tocris Bioschience).

A hydrogel composition of the present disclosure can include a Notch agonist. Examples of suitable Notch agonists include Jagged 1 and Delta 1, or an active fragment or derivative thereof. A suitable Notch agonist is a DSL peptide (Dontu et al., 2004. Breast Cancer Res 6. R605-R615) with the sequence CDDYYYGFGCNKFCRPR (SEQ ID NO:1).

A hydrogel composition of the present disclosure can include an activator of the prostaglandin signalling pathway Such activators include, e.g., Phospholipids, Arachidonic acid (AA), prostaglandin E2 (PGE2), prostaglandin G2 (PGG2), prostaglandin F2 (PGF2), prostaglandin H2 (PGH2), and prostaglandin D2 (PGD2).

A hydrogel composition of the present disclosure can include a RANK ligand.

The pH of a hydrogel composition of the present disclosure can be in the range from about 7.0 to 7.8, in the range from about 7.2 to 7.6, or about 7.4. The pH may be maintained using a buffer. A suitable buffer can readily be selected by the skilled person. Buffers that may be used include carbonate buffers (e.g. NaHCO₃), and phosphates (e.g. NaH₂PO₄). Other buffers such as N-[2-hydroxyethyl]-piperazine-N-[2-ethanesul-phonic acid] (HEPES) and 3-[N-morpholino]-propanesulfonic acid (MOPS) may also be used.

A hydrogel composition of the present disclosure one or more amino acids. Amino acids which may be present include L-alanine, L-arginine, L-asparagine, L-aspartic acid, L-cysteine, L-cystine, L-glutamic acid, L-glutamine, L-glycine, L-histidine, L-isoleucine, L-leucine, L-lysine, L-methionine, L-phenylalanine, L-proline, L-serine, L-threonine, L-tryptophan, L-tyrosine, L-valine and combinations thereof.

A hydrogel composition of the present disclosure can include one or more vitamins. Vitamins which may be present include thiamine (vitamin B1), riboflavin (vitamin B2), niacin (vitamin B3), D-calcium pantothenate (vitamin B5), pyridoxal/pyridoxamine/pyridoxine (vitamin B6), folic acid (vitamin B9), cyanocobalamin (vitamin B12), ascorbic acid (vitamin C), calciferol (vitamin D2), DL-alpha tocopherol (vitamin E), biotin (vitamin H) and menadione (vitamin K).

A hydrogel composition of the present disclosure can include one or more inorganic salts. Inorganic salts that may be present include salts of calcium, copper, iron, magnesium, potassium, sodium, zinc. The salts are normally used in the form of chlorides, phosphates, sulfates, nitrates and bicarbonates.

In some cases, a hydrogel composition of the present disclosure does not include serum, e.g., the hydrogel composition is serum free. In some cases, a hydrogel composition of the present disclosure includes a serum replacement.

A hydrogel composition of the present disclosure can include other components. A hydrogel composition of the present disclosure can include standard culture medium components, such as amino acids, vitamins, inorganic salts, a carbon energy source, and a buffer. Other standard cell culture components that may be included in the culture include hormones, such as progesterone, proteins, such as albumin, catalase, insulin, and transferrin.

A hydrogel composition of the present disclosure can include known cell culture media. The skilled person will understand from common general knowledge the types of culture media that might be used for cell culture, including stem cell culture. Suitable cell culture media are available commercially, and include, but are not limited to, Dulbecco's Modified Eagle Media (DMEM), Minimal Essential Medium (MEM), Knockout-DMEM (KO-DMEM), Glasgow Minimal Essential Medium (G-MEM), Basal Medium Eagle (BME), DMEM/Ham's F12, Advanced DMEM/Ham's F12, Iscove's Modified Dulbecco's Media and Minimal Essential Media (MEM), Ham's F-10, Ham's F-12, Medium 199, and RPMI 1640 Media.

Cells that can be cultured using a method of the present disclosure include mammalian cells. The cells can be undifferentiated cells, such as pluripotent, multipotent, oligopotent or unipotent cells. The cells can be differentiated cells. The cells can be a mix of differentiated and undifferentiated cells. The cells being cultured in a hydrogel composition of the present disclosure can be a single type of cell; or can be a mixture of two or more types of cells.

The cells can be primary cells, genetically modified cells (e.g., genetically modified primary cells), and the like. The cells can be human cells, non-human primate cells, rodent (e.g., mouse; rat) cells, lagomorph (e.g., rabbit) cells, ungulate cells, etc. Cells of any of a variety of cell types can be cultured using a method of the present disclosure. Such cells can include cells from tissue samples, including but not limited to, blood, bone, brain, kidney, muscle, spinal cord, nerve, endocrine system, uterine, ear, foreskin, liver, intestine, bladder or skin. The cells can be obtained from an individual having a particular disease or an individual in need of pluripotent stem cells. The cells can include neural cells, lymphocytes, epidermal cells, intestinal cells, fibroblasts, keratinocytes, adipocytes, cardiomyocytes, pancreatic islet cells, hepatocytes, astrocytes, oligodendrocytes, retinal cells, and the like. The cells can be autologous cells; for example, the cells can be obtained from an individual, and cultured using a method of the present disclosure, whereupon, after culturing (and possible modification, differentiation, etc.), returned to the individual from which the cells were obtained. In some cases, the cells are human cells. In some cases, the cells are rodent (e.g., mouse; rat) cells. In some cases, the cells are non-human primate cells.

### Stem Cells

Cells that can be cultured using a method of the present disclosure include hematopoietic stem cells, embryonic stem cells, mesenchymal stem cells, neural stem cells, epidermal stem cells, endothelial stem cells, gastrointestinal stem cells, liver stem cells, cord blood stem cells, amniotic fluid stem cells, skeletal muscle stem cells, smooth muscle stem cells (e.g., cardiac smooth muscle stem cells), pancreatic stem cells, olfactory stem cells, hematopoietic stem cells, induced pluripotent stem cells; and the like.

In some cases, cells cultured using a method of the present disclosure are stem cells. In some cases, cells cultured using a method of the present disclosure are pluripotent stem cells.

Suitable human embryonic stem (ES) cells include, but are not limited to, any of a variety of available human ES lines, e.g., BG01 (hESBGN-01), BG02 (hESBGN-02), BG03 (hESBGN-03) (BresaGen, Inc.; Athens, Ga.); SA01 (Sahlgrenska 1), SA02 (Sahlgrenska 2) (Cellartis AB; Goeteborg, Sweden); ES01 (HES-1), ES01 (HES-2), ES03 (HES-3), ES04 (HES-4), ES05 (HES-5), ES06 (HES-6) (ES Cell International; Singapore); UC01 (HSF-1), UC06 (HSF-6) (University of California, San Francisco; San Francisco, Calif.); WA01 (H1), WA07 (H7), WA09 (H9), WA13 (H13), WA14 (H14) (Wisconsin Alumni Research Foundation; WARF; Madison, Wis.). Cell line designations are given as the National Institutes of Health (NIH) code, followed in parentheses by the provider code. See, e.g., U.S. Pat. No. 6,875,607. Suitable human ES cell lines can be positive for one, two, three, four, five, six, or all seven of the following markers: stage-specific embryonic antigen-3 (SSEA-3); SSEA-4; TRA 1-60; TRA 1-81; Oct-4; GCTM-2; and alkaline phosphatase.

Hematopoietic stem cells (HSCs) are mesoderm-derived cells that can be isolated from bone marrow, blood, cord blood, fetal liver and yolk sac. HSCs are characterized as CD34⁺ and CD3⁻. HSCs can repopulate the erythroid, neutrophil-macrophage, megakaryocyte and lymphoid hematopoietic cell lineages in vivo. In vitro, HSCs can be induced to undergo at least some self-renewing cell divisions and can be induced to differentiate to the same lineages as is seen in vivo. As such, HSCs can be induced to differentiate into one or more of erythroid cells, megakaryocytes, neutrophils, macrophages, and lymphoid cells.

Neural stem cells (NSCs) are capable of differentiating into neurons, and glia (including oligodendrocytes, and astrocytes). A neural stem cell is a multipotent stem cell which is capable of multiple divisions, and under specific conditions can produce daughter cells which are neural stem cells, or neural progenitor cells that can be neuroblasts or glioblasts, e.g., cells committed to become one or more types of neurons and glial cells respectively. Methods of obtaining NSCs are known in the art. In some cases, NSCs cultured in the hydrogel composition remain multipotent after multiple passages.

Mesenchymal stem cells (MSC), originally derived from the embryonal mesoderm and isolated from adult bone marrow, can differentiate to form muscle, bone, cartilage, fat, marrow stroma, and tendon. Methods of isolating MSC are known in the art; and any known method can be used to obtain MSC. See, e.g., U.S. Pat. No. 5,736,396, which describes isolation of human MSC.

An induced pluripotent stem (iPS) cell is a pluripotent stem cell induced from a somatic cell, e.g., a differentiated somatic cell. iPS cells are capable of self-renewal and differentiation into cell fate-committed stem cells, including neural stem cells, as well as various types of mature cells.

iPS cells can be generated from somatic cells, including skin fibroblasts, using, e.g., known methods. iPS cells produce and express on their cell surface one or more of the following cell surface antigens: SSEA-3, SSEA-4, TRA-1-60, TRA-1-81, TRA-2-49/6E, and Nanog. In some embodiments, iPS cells produce and express on their cell surface SSEA-3, SSEA-4, TRA-1-60, TRA-1-81, TRA-2-49/6E, and Nanog. iPS cells express one or more of the following genes: Oct-3/4, Sox2, Nanog, GDF3, REX1, FGF4, ESG1, DPPA2, DPPA4, and hTERT. In some embodiments, an iPS cell expresses Oct-3/4, Sox2, Nanog, GDF3, REX1, FGF4, ESG1, DPPA2, DPPA4, and hTERT. Methods of generating iPS are known in the art, and any such method can be used to generate iPS. See, e.g., Takahashi and Yamanaka (2006) Cell 126:663-676; Yamanaka et. al. (2007) Nature 448:313-7; Wernig et al. (2007) Nature 448:318-24; Maherali (2007) Cell Stem Cell 1:55-70; Nakagawa et al. (2008) Nat. Biotechnol. 26:101; Takahashi et al. (2007) Cell 131:861; Takahashi et al. (2007) Nat. Protoc. 2:3081; and Okita et al. (2007 Nature 448:313.

iPS cells can be generated from somatic cells (e.g., skin fibroblasts) by genetically modifying the somatic cells with one or more expression constructs encoding Oct-3/4 and Sox2. In some embodiments, somatic cells are genetically modified with one or more expression constructs comprising nucleotide sequences encoding Oct-3/4, Sox2, c-myc, and Klf4. In some embodiments, somatic cells are genetically modified with one or more expression constructs comprising nucleotide sequences encoding Oct-4, Sox2, Nanog, and LIN28.

In some cases, cells cultured using a method of the present disclosure are somatic stem cells (also known as “adult stem cells”). Suitable somatic stem cells include, e.g., tissue stem cells; and tissue precursor cells. Stem cells that can be cultured in a hydrogel composition of the present disclosure include, e.g., neural stem cells, hematopoietic stem cells, mammary stem cells, epidermal stem cells, intestinal stem cells, mesenchymal stem cells, endothelial stem cells, pancreatic stem cells, dermal stem cells, myocardial stem cells, oligodendrocyte precursor cells, neural stem cells, olfactory adult stem cells, neural crest stem cells, hepatic stem cells, and the like.

### Immune Cells

Cells that can be cultured using a method of the present disclosure include immune cells. Immune cells include, e.g., T cells, natural killer (NK) cells, B cells, and the like. T cells include CD4⁺ T cells, CD8⁺ T cells, regulatory T cells (Tregs), and the like. In some cases, the cells are genetically modified with one or more nucleic acids comprising nucleotide sequences encoding proteins of interest. For example, a T cell can be genetically modified with a nucleic acid comprising a nucleotide sequence encoding a chimeric antigen receptor.

### Methods of Differentiating Cells

The present disclosure provides methods of producing differentiated cells from a stem cell or a precursor cell, the methods comprising culturing a stem cell or precursor cell in a hydrogel composition of the present disclosure, for a period of time and under conditions suitable for inducing differentiation of the stem cell or precursor cell. Conditions for inducing differentiation of a stem cell or precursor cell depend in part on the desired differentiated cell. Conditions can include inclusion in the hydrogel of one or more factors that induce differentiation.

### Methods of Isolating Cells

The present disclosure provides methods of producing a stem cell, a precursor cell, or a differentiated cell, the methods comprising: a) culturing a cell in a hydrogel composition of the present disclosure; and b) isolating the cell from the hydrogel composition. For example, in some cases, a cell is cultured in a hydrogel composition of the present disclosure at a temperature at which the hydrogel is a semi-solid (e.g., a gel) (e.g., 37° C.); and the cell, or progeny of the cell, is isolated from the hydrogel composition by reducing the temperature (e.g., to about 4° C.) of the hydrogel composition such that the hydrogel composition becomes a liquid. A cell can be isolated from a liquid form of the hydrogel composition using centrifugation or any other means.

In some cases, a method of the present disclosure comprises: a) culturing a stem cell in a hydrogel composition of the present disclosure at a temperature at which the hydrogel is a semi-solid (e.g., a gel), where the hydrogel composition comprises one or more factors that induce differentiation of the stem cell; b) reducing the temperature of the hydrogel composition such that the hydrogel composition becomes a liquid; and c) isolating the differentiated cell(s) from the liquid.

In some cases, a method of the present disclosure comprises: a) culturing a stem cell in a hydrogel composition of the present disclosure at a temperature at which the hydrogel is a semi-solid (e.g., a gel), where the hydrogel composition comprises one or more factors that promote growth and proliferation of the stem cell; b) reducing the temperature of the hydrogel composition such that the hydrogel composition becomes a liquid; and c) isolating the proliferated stem cells from the liquid.

### Treatment Methods

The present disclosure provides methods of treating a disease or disorder in an individual in need thereof. In some cases, the methods involve culturing cells using a method of the present disclosure, as described above; isolating the cells; and administering to the individual the isolated cells. In some cases, the methods involve implanting into the individual a thermoreversible polymer-cell composition of the present disclosure.

Diseases that can be treated using cells cultured in a thermoreversible polymer of the present disclosure, or using a thermoreversible polymer-cell composition of the present disclosure, include, but are not limited to, autoimmune disease; diseases for which treatment involves regeneration of neural cells/tissue; diseases for which treatment involves regeneration of cardiac cells/tissues; Parkinson's Disease; and Alzheimer's Disease. Cells differentiated from the stem cells using a method of the present disclosure include myocardial cells, insulin-producing cells, neuronal cells, oligodendrocytes, and the like; such cells can be safely utilized in stem cell transplantation therapies for treatment of various diseases such as heart failure, insulin dependent diabetes mellitus, Parkinson's disease and spinal cord injury. Stem cells, or differentiated cells derived therefrom, can be used for autologous cells therapy, wherein the therapy is specific (e.g., personalized) for a particular subject. Stem cells, or differentiated cells derived therefrom, can be used for or non-autologous therapy.

Subjects suitable for treatment with a subject method include individuals who have been diagnosed as having a blood cell cancer (e.g., a leukemia); individuals who have been diagnosed with AIDS; individuals with sickle cell anemia; individuals with an immune disorder, e.g., an acquired immunodeficiency, a genetic immunodeficiency; individuals with Type 1 diabetes; individuals with a nervous system disorder such as Alzheimer's disease, Parkinson's disease, Huntington's disease, Lou Gehrig's disease, spinal cord injury, stroke, etc.; individuals with a liver disorder such as hepatitis, cirrhosis, a metabolic disorder affecting the liver or central nervous system (e.g., lysosomal storage disease); individuals with a disorder of the cartilage or bone, e.g., individuals requiring joint replacement, individuals with osteoarthritis, individuals with osteoporosis, etc.; individuals with a cardiac disorder, e.g., myocardial infarction, coronary artery disease, or other disorder resulting in ischemic cardiac tissue; individuals with renal disorders, e.g., kidney failure (e.g., individuals on kidney dialysis); individuals with skeletal muscle disorders, such as muscular dystrophy; and individuals with a lung disorder such as emphysema, pulmonary fibrosis, idiopathic pulmonary fibrosis, etc.

### Utility

The subject thermoreversible polymers, hydrogels and methods find use in a variety of applications. Applications of interest include, but are not limited to, applications where the culturing and/or differentiation of cells are of interest. Protocols of interest can use single cells or small aggregates of stem cells and evenly disperse them throughout the hydrogel material at cold temperatures. The material can then either be spread out onto a two-dimensional surface or dropped into warm media in a stirred tank reactor. Upon warming to 37° C., the material can gel and encapsulate the cells. After changing media every day or every other day and checking progress of cell growth, the materials can be cooled and centrifuged to isolate the cells.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

Aspect 1. A thermoreversible polymer comprising: a) a N-isopropylacrylamide (NIPAM) co-monomer; b) a lower alkyl amine co-monomer; and c) a poly(ethylene glycol) (PEG) co-monomer, wherein the terminal PEG monomer is substituted with alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl.

Aspect 2. The thermoreversible polymer of aspect 1, wherein: i) the lower alkyl amine co-monomer comprises n-butyl, isobutyl, tert-butyl, n-propyl, pentyl, isopropyl, or isopentyl; and ii) the terminal PEG monomer is substituted with an alkoxy group.

Aspect 3. The thermoreversible polymer of aspect 2, wherein the alkoxy group is an C1-C6 alkoxy selected from the group consisting of methoxy, ethoxy, n-propoxy, isoproxy, n-butoxy, isobutoxy, tert-butoxy, pentoxy and isopentoxy.

Aspect 4. The thermoreversible polymer of any one of aspects 1-3, comprising the formula (I):

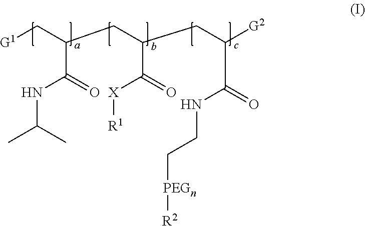

wherein:
a, b, and c are molar fractions of the co-monomers, where a, b, and c are each greater than zero;
PEGₙ is a polyethyleneglycol polymer and n is an integer from 1 to 2500;
X is independently selected from C, O or NH;
R¹ is an alkyl or a substituted alkyl;
R² is alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl; and
G¹ and G² are each independently selected from a polymer segment, a terminal group, a linker and a linked modifying agent.

Aspect 5. The thermoreversible polymer of aspect 4, wherein R¹ is a C1-C6 alkyl.

Aspect 6. The thermoreversible polymer of aspect 5, wherein R¹ is an alkyl group selected from the group consisting of n-butyl, isobutyl, tert-butyl, n-propyl, pentyl, isopropyl and isopentyl.

Aspect 7. The thermoreversible polymer of aspect 6, wherein R¹ is n-butyl.

Aspect 8. The thermoreversible polymer of any one of aspects 4-7, wherein R² is an alkoxy group.

Aspect 9. The thermoreversible polymer of aspect 8, wherein R² is a C1-C6 alkoxy group selected from the group consisting of methoxy, ethoxy, n-propoxy, isoproxy, n-butoxy, isobutoxy, tert-butoxy, pentoxy and isopentoxy.

Aspect 10. The thermoreversible polymer of aspect 9, wherein R² is methoxy.

Aspect 11. The thermoreversible polymer of any one of aspects 2-10, wherein a>0.8; 0.2>b>0; and 0.1>c>0.

Aspect 12. The thermoreversible polymer of any one of aspects 1-11, comprising the formula II:

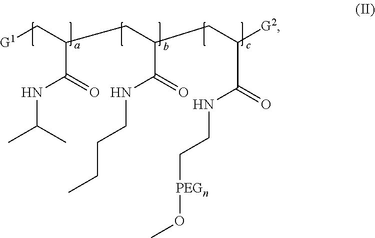

wherein n is 1 to 25; and
G¹ and G² are each independently selected from a polymer segment, a terminal group, a linker and a linked modifying agent.

Aspect 13. The thermoreversible polymer of any one of aspects 1-12, wherein the PEG or PEGₙ has a MW of 2 kDa to 100 kDa.

Aspect 14. The thermoreversible polymer of any one of aspects 1-3, comprising the formula (III):

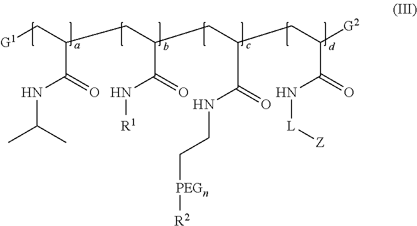

wherein:
a, b, c, and d are molar fractions of the co-monomers, where a, b, c and d are each greater than zero;
PEGₙ is a polyethyleneglycol polymer and n is an integer from 1 to 2500;
R¹ is an alkyl or a substituted alkyl;
R² is alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl;
L is a linker;
Z² is a modifying agent; and
G¹ and G² are each independently selected from a polymer segment, a terminal group, a linker and a linked modifying agent.

Aspect 15. The thermoreversible polymer of aspect 14, wherein R¹ is a C1-C6 alkyl.

Aspect 16. The thermoreversible polymer of aspect 15, wherein R¹ is an alkyl group selected from the group consisting of n-butyl, isobutyl, tert-butyl, n-propyl, pentyl, isopropyl and isopentyl.

Aspect 17. The thermoreversible polymer of aspect 16, wherein R¹ is n-butyl.

Aspect 18. The thermoreversible polymer of any one of aspects 14-17, wherein R² is an alkoxy group.

Aspect 19. The thermoreversible polymer of aspect 18, wherein R² is a C1-C6 alkoxy group selected from the group consisting of methoxy, ethoxy, n-propoxy, isoproxy, n-butoxy, isobutoxy, tert-butoxy, pentoxy and isopentoxy.

Aspect 20. The thermoreversible polymer of aspect 19, wherein R² is methoxy.

Aspect 21. The thermoreversible polymer of any one of aspects 14-20, wherein Z² is a chemoselective functional group selected from a thiol, an alkyne, a cyclooctyne, an azide, a phosphine, a maleimide, an alkoxyamine, an aldehyde and protected versions or precursors thereof.

Aspect 22. The thermoreversible polymer of any one of aspects 14-20, wherein Z² is a modifying agent selected from a heparin, a hyaluronic acid, a specific binding member, a peptide, a nucleic acid, gelatin, fibronectin, collagen, laminin, basis fibroblast growth factor (bFGF), epidermal growth factor (EGF), insulin, progesterone, glucose, stromal cell derived factor-1 (SDF-1), thymosin beta-4, sonic hedgehog (SHH), Noggin, Activin, transforming growth factor-β (TGF-β) (TGFβ3), FGF8, brain-derived neurotrophic factor (BDNF), glial cell-derived neurotrophic factor (GDNF), neurotrophic factor-3 (NT3), platelet-derived growth factor (PDGF), and insulin-like growth factor-1 (IGF-1).

Aspect 23. The thermoreversible polymer of any one of aspects 2-10, wherein a>0.8; 0.2>b>0; and 0.1>c>0.

Aspect 24. The thermoreversible polymer of any one of aspects 4-23, wherein one or more of G¹, G² and Z² are independently selected a modifying agent selected from a heparin, a hyaluronic acid, a member of a specific binding pair, a polypeptide, and a nucleic acid.

Aspect 25. The thermoreversible polymer of any one of aspects 4-23, wherein one or more of G¹, G² and Z² are independently selected a modifying agent selected from gelatin, fibronectin, collagen, or laminin.

Aspect 26. The thermoreversible polymer of any one of aspects 4-23, wherein one or more of G¹, G² and Z² is a polypeptide selected from a chemokine, a peptide hormone, or a growth factor.

Aspect 27. The thermoreversible polymer of aspect 26, wherein the polypeptide is fibroblast growth factor, epidermal growth factor, hepatic growth factor insulin, stromal cell-derived factor-1, thymosin beta-4, sonic hedgehog, Noggin, activin, transforming growth factor, bone morphogenic protein, brain-derived neurotrophic factor, glial cell-derived neurotrophic factor, neurotrophin-3, platelet-derived growth factor, FGF-2, FGF-8, keratinocyte growth factor, or insulin-like growth factor.

Aspect 28. The thermoreversible polymer of aspect 26, wherein the polypeptide is selected from hepatic growth factor; bone morphogenic protein; FGF-2; FGF-8, and keratinocyte growth factor.

Aspect 29. The thermoreversible polymer of any one of aspects 1-3, comprising the formula (IV):

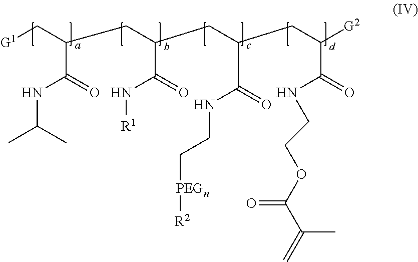

wherein:
a, b, c, and d are molar fractions of the co-monomers, where a, b, c and d are each greater than zero;
PEGₙ is a polyethyleneglycol polymer and n is an integer from 1 to 2500;
R¹ is an alkyl or a substituted alkyl;
R² is alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl; and
G¹ and G² are each independently selected from a polymer segment, a terminal group, a linker and a linked modifying agent.

Aspect 30. The thermoreversible polymer of aspect 29, wherein R¹ is a C1-C6 alkyl.

Aspect 31. The thermoreversible polymer of aspect 30, wherein R¹ is an alkyl group selected from the group consisting of n-butyl, isobutyl, tert-butyl, n-propyl, pentyl, isopropyl and isopentyl.

Aspect 32. The thermoreversible polymer of aspect 31, wherein R¹ is n-butyl.

Aspect 33. The thermoreversible polymer of any one of aspects 30-32, wherein R² is an alkoxy group.

Aspect 34. The thermoreversible polymer of aspect 33, wherein R² is a C1-C6 alkoxy group selected from the group consisting of methoxy, ethoxy, n-propoxy, isoproxy, n-butoxy, isobutoxy, tert-butoxy, pentoxy and isopentoxy.

Aspect 35. The thermoreversible polymer of aspect 34, wherein R² is methoxy.

Aspect 36. The thermoreversible polymer of any one of aspects 1-3, comprising the formula (V):

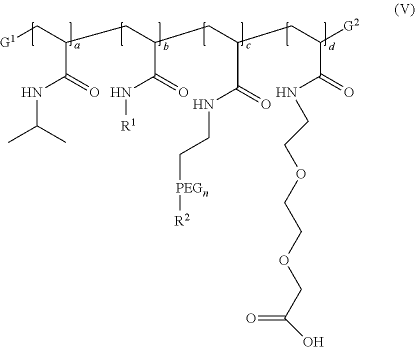

wherein:
a, b, c, and d are molar fractions of the co-monomers, where a, b, c and d are each greater than zero;
PEGₙ is a polyethyleneglycol polymer and n is an integer from 1 to 2500;
R¹ is an alkyl or a substituted alkyl;
R² is alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl; and
G¹ and G² are each independently selected from a polymer segment, a terminal group, a linker and a linked modifying agent.

Aspect 37. The thermoreversible polymer of aspect 36, wherein R¹ is a C1-C6 alkyl.

Aspect 38. The thermoreversible polymer of aspect 37, wherein R¹ is an alkyl group selected from the group consisting of n-butyl, isobutyl, tert-butyl, n-propyl, pentyl, isopropyl and isopentyl.

Aspect 39. The thermoreversible polymer of aspect 38, wherein R¹ is n-butyl.

Aspect 40. The thermoreversible polymer of any one of aspects 37-39, wherein R² is an alkoxy group.

Aspect 41. The thermoreversible polymer of aspect 40, wherein R² is a C1-C6 alkoxy group selected from the group consisting of methoxy, ethoxy, n-propoxy, isoproxy, n-butoxy, isobutoxy, tert-butoxy, pentoxy and isopentoxy.

Aspect 42. The thermoreversible polymer of aspect 41, wherein R² is methoxy.

Aspect 43. The thermoreversible polymer of any one of aspects 1-3, comprising the formula (VI):

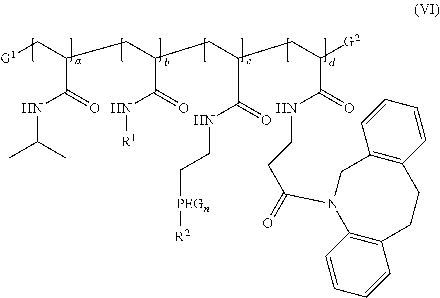

wherein:
a, b, c, and d are molar fractions of the co-monomers, where a, b, c and d are each greater than zero;
PEGₙ is a polyethyleneglycol polymer and n is an integer from 1 to 2500;
R¹ is an alkyl or a substituted alkyl;
R² is alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl; and
G¹ and G² are each independently selected from a polymer segment, a terminal group, a linker and a linked modifying agent.

Aspect 44. The thermoreversible polymer of aspect 43, wherein R¹ is a C1-C6 alkyl.

Aspect 45. The thermoreversible polymer of aspect 44, wherein R¹ is an alkyl group selected from the group consisting of n-butyl, isobutyl, tert-butyl, n-propyl, pentyl, isopropyl and isopentyl.

Aspect 46. The thermoreversible polymer of aspect 45, wherein R¹ is n-butyl.

Aspect 47. The thermoreversible polymer of any one of aspects 44-46, wherein R² is an alkoxy group.

Aspect 48. The thermoreversible polymer of aspect 47, wherein R² is a C1-C6 alkoxy group selected from the group consisting of methoxy, ethoxy, n-propoxy, isoproxy, n-butoxy, isobutoxy, tert-butoxy, pentoxy and isopentoxy.

Aspect 49. The thermoreversible polymer of aspect 48, wherein R² is methoxy.

Aspect 50. The thermoreversible polymer of any one of aspects 1-3, comprising the formula (VII):

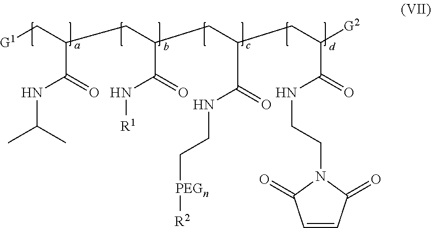

wherein:
a, b, c, and d are molar fractions of the co-monomers, where a, b, c and d are each greater than zero;
PEGₙ is a polyethyleneglycol polymer and n is an integer from 1 to 2500;
R¹ is an alkyl or a substituted alkyl;
R² is alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl; and
G¹ and G² are each independently selected from a polymer segment, a terminal group, a linker and a linked modifying agent.

Aspect 51. The thermoreversible polymer of aspect 50, wherein R¹ is a C1-C6 alkyl.

Aspect 52. The thermoreversible polymer of aspect 51, wherein R¹ is an alkyl group selected from the group consisting of n-butyl, isobutyl, tert-butyl, n-propyl, pentyl, isopropyl and isopentyl.

Aspect 53. The thermoreversible polymer of aspect 52, wherein R¹ is n-butyl.

Aspect 54. The thermoreversible polymer of any one of aspects 51-53, wherein R² is an alkoxy group.

Aspect 55. The thermoreversible polymer of aspect 54, wherein R² is a C1-C6 alkoxy group selected from the group consisting of methoxy, ethoxy, n-propoxy, isoproxy, n-butoxy, isobutoxy, tert-butoxy, pentoxy and isopentoxy.

Aspect 56. The thermoreversible polymer of aspect 55, wherein R² is methoxy.

Aspect 57. The thermoreversible polymer of any one of aspects 1-3, comprising the formula (VIII):

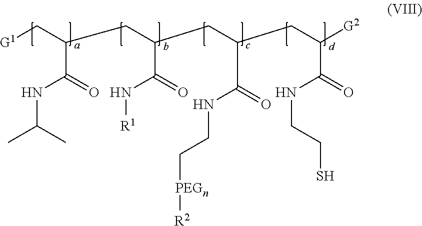

wherein:
a, b, c, and d are molar fractions of the co-monomers, where a, b, c and d are each greater than zero;
PEGₙ is a polyethyleneglycol polymer and n is an integer from 1 to 2500;
R¹ is an alkyl or a substituted alkyl;
R² is alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl; and
G¹ and G² are each independently selected from a polymer segment, a terminal group, a linker and a linked modifying agent.

Aspect 58. The thermoreversible polymer of aspect 57, wherein R¹ is a C1-C6 alkyl.

Aspect 59. The thermoreversible polymer of aspect 58, wherein R¹ is an alkyl group selected from the group consisting of n-butyl, isobutyl, tert-butyl, n-propyl, pentyl, isopropyl and isopentyl.

Aspect 60. The thermoreversible polymer of aspect 59, wherein R¹ is n-butyl.

Aspect 61. The thermoreversible polymer of any one of aspects 58-60, wherein R² is an alkoxy group.

Aspect 62. The thermoreversible polymer of aspect 61, wherein R² is a C1-C6 alkoxy group selected from the group consisting of methoxy, ethoxy, n-propoxy, isoproxy, n-butoxy, isobutoxy, tert-butoxy, pentoxy and isopentoxy.

Aspect 63. The thermoreversible polymer of aspect 62, wherein R² is methoxy.

Aspect 64. The thermoreversible polymer of any one of aspects 1-3, comprising the formula (IX):

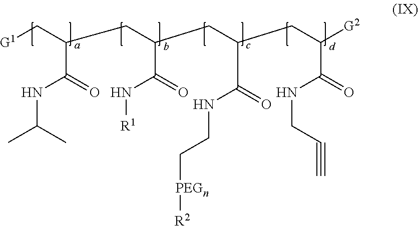

wherein:
a, b, c, and d are molar fractions of the co-monomers, where a, b, c and d are each greater than zero;
PEGₙ is a polyethyleneglycol polymer and n is an integer from 1 to 2500;
R¹ is an alkyl or a substituted alkyl;
R² is alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl; and
G¹ and G² are each independently selected from a polymer segment, a terminal group, a linker and a linked modifying agent.

Aspect 65. The thermoreversible polymer of aspect 64, wherein R¹ is a C1-C6 alkyl.

Aspect 66. The thermoreversible polymer of aspect 65, wherein R¹ is an alkyl group selected from the group consisting of n-butyl, isobutyl, tert-butyl, n-propyl, pentyl, isopropyl and isopentyl.

Aspect 67. The thermoreversible polymer of aspect 66, wherein R¹ is n-butyl.

Aspect 68. The thermoreversible polymer of any one of aspects 65-67, wherein R² is an alkoxy group.

Aspect 69. The thermoreversible polymer of aspect 68, wherein R² is a C1-C6 alkoxy group selected from the group consisting of methoxy, ethoxy, n-propoxy, isoproxy, n-butoxy, isobutoxy, tert-butoxy, pentoxy and isopentoxy.

Aspect 70. The thermoreversible polymer of aspect 69, wherein R² is methoxy.

Aspect 71. The thermoreversible polymer of any one of aspects 1-3, comprising the formula (X):

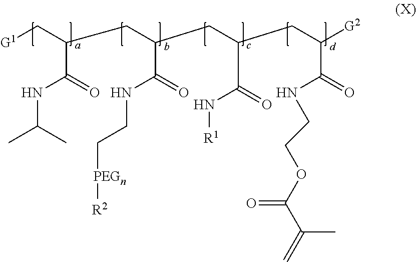

wherein:
a, b, c, and d are molar fractions of the co-monomers, where a, b, c and d are each greater than zero;
PEGₙ is a polyethyleneglycol polymer and n is an integer from 1 to 2500;
R¹ is an alkyl or a substituted alkyl;
R² is alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl; and
G¹ and G² are each independently selected from a polymer segment, a terminal group, a linker and a linked modifying agent.

Aspect 72. The thermoreversible polymer of aspect 71, wherein R¹ is a C1-C6 alkyl.

Aspect 73. The thermoreversible polymer of aspect 72, wherein R¹ is an alkyl group selected from the group consisting of n-butyl, isobutyl, tert-butyl, n-propyl, pentyl, isopropyl and isopentyl.

Aspect 74. The thermoreversible polymer of aspect 73, wherein R¹ is n-butyl.

Aspect 75. The thermoreversible polymer of any one of aspects 72-74, wherein R² is an alkoxy group.

Aspect 76. The thermoreversible polymer of aspect 75, wherein R² is a C1-C6 alkoxy group selected from the group consisting of methoxy, ethoxy, n-propoxy, isoproxy, n-butoxy, isobutoxy, tert-butoxy, pentoxy and isopentoxy.

Aspect 77. The thermoreversible polymer of aspect 76, wherein R² is methoxy.

Aspect 78. The thermoreversible polymer of any one of aspects 1-3, comprising the formula (XI):

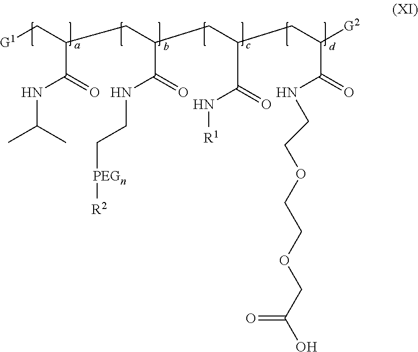

wherein:
a, b, c, and d are molar fractions of the co-monomers, where a, b, c and d are each greater than zero;
PEGₙ is a polyethyleneglycol polymer and n is an integer from 1 to 2500;
R¹ is an alkyl or a substituted alkyl;
R² is alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl; and
G¹ and G² are each independently selected from a polymer segment, a terminal group, a linker and a linked modifying agent.

Aspect 79. The thermoreversible polymer of aspect 78, wherein R¹ is a C1-C6 alkyl.

Aspect 80. The thermoreversible polymer of aspect 79, wherein R¹ is an alkyl group selected from the group consisting of n-butyl, isobutyl, tert-butyl, n-propyl, pentyl, isopropyl and isopentyl.

Aspect 81. The thermoreversible polymer of aspect 80, wherein R¹ is n-butyl.

Aspect 82. The thermoreversible polymer of any one of aspects 79-81, wherein R² is an alkoxy group.

Aspect 83. The thermoreversible polymer of aspect 82, wherein R² is a C1-C6 alkoxy group selected from the group consisting of methoxy, ethoxy, n-propoxy, isoproxy, n-butoxy, isobutoxy, tert-butoxy, pentoxy and isopentoxy.

Aspect 84. The thermoreversible polymer of aspect 83, wherein R² is methoxy.

Aspect 85. The thermoreversible polymer of any one of aspects 1-3, comprising the formula (XII):

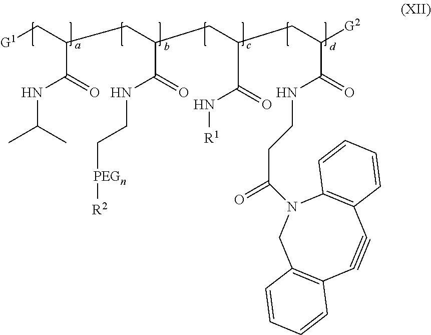

wherein:
a, b, c, and d are molar fractions of the co-monomers, where a, b, c and d are each greater than zero;
PEGₙ is a polyethyleneglycol polymer and n is an integer from 1 to 2500;
R¹ is an alkyl or a substituted alkyl;
R² is alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl; and
G¹ and G² are each independently selected from a polymer segment, a terminal group, a linker and a linked modifying agent.

Aspect 86. The thermoreversible polymer of aspect 85, wherein R¹ is a C1-C6 alkyl.

Aspect 87. The thermoreversible polymer of aspect 86, wherein R¹ is an alkyl group selected from the group consisting of n-butyl, isobutyl, tert-butyl, n-propyl, pentyl, isopropyl and isopentyl.

Aspect 88. The thermoreversible polymer of aspect 87, wherein R¹ is n-butyl.

Aspect 89. The thermoreversible polymer of any one of aspects 86-88, wherein R² is an alkoxy group.

Aspect 90. The thermoreversible polymer of aspect 89, wherein R² is a C1-C6 alkoxy group selected from the group consisting of methoxy, ethoxy, n-propoxy, isoproxy, n-butoxy, isobutoxy, tert-butoxy, pentoxy and isopentoxy.

Aspect 91. The thermoreversible polymer of aspect 90, wherein R² is methoxy.

Aspect 92. The thermoreversible polymer of any one of aspects 1-3, comprising the formula (XIII):

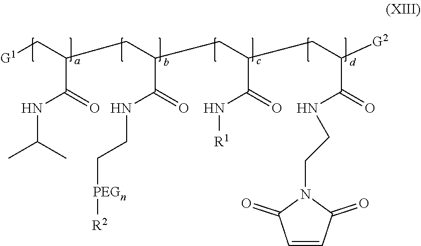

wherein
a, b, c, and d are molar fractions of the co-monomers, where a, b, c and d are each greater than zero;
PEGₙ is a polyethyleneglycol polymer and n is an integer from 1 to 2500;
R¹ is an alkyl or a substituted alkyl;
R² is alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl; and
G¹ and G² are each independently selected from a polymer segment, a terminal group, a linker and a linked modifying agent.

Aspect 93. The thermoreversible polymer of aspect 92, wherein R¹ is a C1-C6 alkyl.

Aspect 94. The thermoreversible polymer of aspect 93, wherein R′ is an alkyl group selected from the group consisting of n-butyl, isobutyl, tert-butyl, n-propyl, pentyl, isopropyl and isopentyl.

Aspect 95. The thermoreversible polymer of aspect 94, wherein R¹ is n-butyl.

Aspect 96. The thermoreversible polymer of any one of aspects 93-95, wherein R² is an alkoxy group.

Aspect 97. The thermoreversible polymer of aspect 96, wherein R² is a C1-C6 alkoxy group selected from the group consisting of methoxy, ethoxy, n-propoxy, isoproxy, n-butoxy, isobutoxy, tert-butoxy, pentoxy and isopentoxy.

Aspect 98. The thermoreversible polymer of aspect 97, wherein R² is methoxy.

Aspect 99. The thermoreversible polymer of any one of aspects 1-3, comprising the formula (XIV):

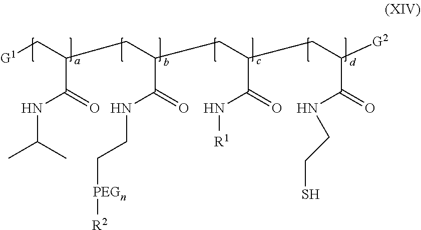

wherein:
a, b, c, and d are molar fractions of the co-monomers, where a, b, c and d are each greater than zero;
PEGₙ is a polyethyleneglycol polymer and n is an integer from 1 to 2500;
R¹ is an alkyl or a substituted alkyl;
R² is alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl; and
G¹ and G² are each independently selected from a polymer segment, a terminal group, a linker and a linked modifying agent.

Aspect 100. The thermoreversible polymer of aspect 99, wherein R¹ is a C1-C6 alkyl.

Aspect 101. The thermoreversible polymer of aspect 100, wherein R¹ is an alkyl group selected from the group consisting of n-butyl, isobutyl, tert-butyl, n-propyl, pentyl, isopropyl and isopentyl.

Aspect 102. The thermoreversible polymer of aspect 101, wherein R¹ is n-butyl.

Aspect 103. The thermoreversible polymer of any one of aspects 100-102, wherein R² is an alkoxy group.

Aspect 104. The thermoreversible polymer of aspect 103, wherein R² is a C1-C6 alkoxy group selected from the group consisting of methoxy, ethoxy, n-propoxy, isoproxy, n-butoxy, isobutoxy, tert-butoxy, pentoxy and isopentoxy.

Aspect 105. The thermoreversible polymer of aspect 104, wherein R² is methoxy.

Aspect 106. The thermoreversible polymer of any one of aspects 1-3, comprising the formula (XV):

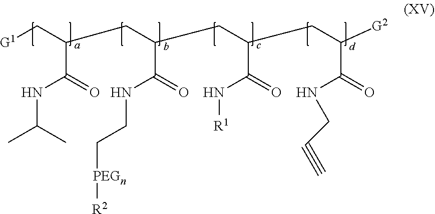

wherein:
a, b, c, and d are molar fractions of the co-monomers, where a, b, c and d are each greater than zero;
PEGₙ is a polyethyleneglycol polymer and n is an integer from 1 to 2500;
R¹ is an alkyl or a substituted alkyl;
R² is alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl; and
G¹ and G² are each independently selected from a polymer segment, a terminal group, a linker and a linked modifying agent.

Aspect 107. The thermoreversible polymer of aspect 106, wherein R¹ is a C1-C6 alkyl.

Aspect 108. The thermoreversible polymer of aspect 107, wherein R¹ is an alkyl group selected from the group consisting of n-butyl, isobutyl, tert-butyl, n-propyl, pentyl, isopropyl and isopentyl.

Aspect 109. The thermoreversible polymer of aspect 108, wherein R¹ is n-butyl.

Aspect 110. The thermoreversible polymer of any one of aspects 107-109, wherein R² is an alkoxy group.

Aspect 111. The thermoreversible polymer of aspect 110, wherein R² is a C1-C6 alkoxy group selected from the group consisting of methoxy, ethoxy, n-propoxy, isoproxy, n-butoxy, isobutoxy, tert-butoxy, pentoxy and isopentoxy.

Aspect 112. The thermoreversible polymer of aspect 111, wherein R² is methoxy.

Aspect 116. The thermoreversible polymer of any one of aspects 1-115, wherein the thermoreversible polymer is a solid at 20° C. or more.

Aspect 117. The thermoreversible polymer of any one of aspects 1-115, wherein the thermoreversible polymer is a solid at 37° C.

Aspect 118. The thermoreversible polymer of any one of aspects 1-115, wherein the thermoreversible polymer is a liquid at 30° C. or less.

Aspect 119. The thermoreversible polymer of any one of aspects 1-115, wherein the thermoreversible polymer is a liquid at 4° C.

Aspect 120. A method of making a thermoreversible polymer, the method comprising: a) co-polymerizing N-isopropylacryliamide and N-acryloxysuccinimide to generate a first copolymer comprising an acrylic backbone; b) contacting the copolymer with an alkyl amine and an alkoxy-polyethylene glycol amine to generate a second copolymer; and c) contacting the second copolymer with isopropylamine to generate a polymer of formula I:

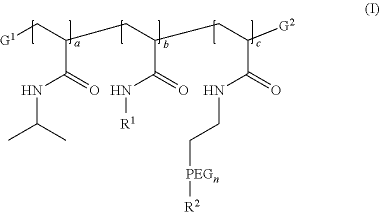

wherein:
a, b and c are molar fractions of the co-monomers, where a, b and c are each greater than zero;
PEGₙ is a polyethyleneglycol polymer and n is an integer from 1 to 2500.
X is independently selected from C, O, and NH.
R¹ is an alkyl or a substituted alkyl;
R² is alkyl, substituted alkyl, hetero; alkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl; and
G¹ and G² are each independently selected from a polymer segment, a terminal group, a linker and a linked modifying agent.

Aspect 121. The method of aspect 120, wherein R¹ is a C1-C6 alkyl.

Aspect 122. The method of aspect 121, wherein R¹ is an alkyl group selected from the group consisting of n-butyl, isobutyl, tert-butyl, n-propyl, pentyl, isopropyl and isopentyl.

Aspect 123. The method of aspect 122, wherein R¹ is n-butyl.

Aspect 124. The method of any one of aspects 121-123, wherein R² is an alkoxy group.

Aspect 125. The method of aspect 124, wherein R² is a C1-C6 alkoxy group selected from the group consisting of methoxy, ethoxy, n-propoxy, isoproxy, n-butoxy, isobutoxy, tert-butoxy, pentoxy and isopentoxy.

Aspect 126. The method of aspect 125, wherein R² is methoxy.

Aspect 127. The method of any one of aspects 120-126, wherein a>0.8; 0.1>b>0; and 0.2>c>0.

Aspect 128. The method of any one of aspects 120-127, wherein the method comprises: a) co-polymerizing N-isopropylacrylamide and N-acryloxysuccinimide to generate a first copolymer comprising an acrylic backbone; b) contacting the copolymer with butylamine and methoxy-polyethylene glycol amine to generate a second copolymer; and c) contacting the second copolymer with isopropylamine to generate a polymer of formula II:

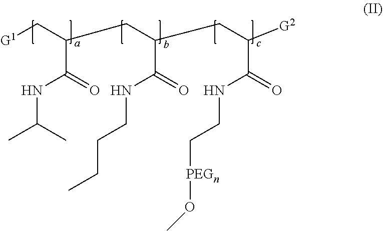

wherein n is an integer from 1 to 2500; and
G¹ and G² are each independently selected from a polymer segment, a terminal group, a linker and a linked modifying agent.

Aspect 129. A method of making a thermoreversible polymer, the method comprising: co-polymerizing N-isopropylacrylamide, N-acryloxysuccinimide and an alkyl methacrylate to generate a first copolymer comprising an acrylic backbone; contacting the copolymer with an alkoxy-polyethylene glycol amine to generate a second copolymer; and contacting the second copolymer with isopropylamine to generate a polymer of formula I:

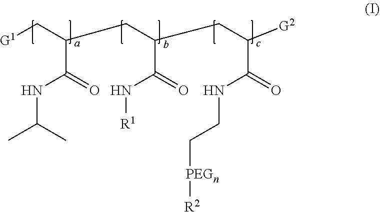

wherein:
a, b and c are molar fractions of the co-monomers, where a, b and c are each greater than zero;
PEGₙ is a polyethyleneglycol polymer and n is an integer from 1 to 2500.
R¹ is an alkyl or a substituted alkyl;
R² is alkyl, substituted alkyl, hetero; alkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl; and
G¹ and G² are each independently selected from a polymer segment, a terminal group, a linker and a linked modifying agent.

Aspect 130. A method of making a thermoreversible polymer, the method comprising: co-polymerizing N-isopropylacrylamide and N-acryloxysuccinimide to generate a first copolymer comprising an acrylic backbone; contacting the copolymer with an alkyl amine (e.g., butyl amine) and an alkoxy-polyethylene glycol amine to generate a second copolymer; contacting the second copolymer with an aminoalkyl methacrylate (e.g., 2-amino methacrylate) to generate a third copolymer and contacting the third copolymer with isopropylamine to generate a polymer of formula IV:

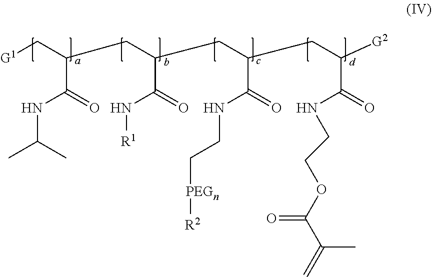

wherein:
a, b and c are molar fractions of the co-monomers, where a, b and c are each greater than zero;
PEGₙ is a polyethyleneglycol polymer and n is an integer from 1 to 2500.
R¹ is an alkyl or a substituted alkyl;
R² is alkyl, substituted alkyl, hetero; alkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl; and
G¹ and G² are each independently selected from a polymer segment, a terminal group, a linker and a linked modifying agent.

Aspect 131. A method of making a thermoreversible polymer, the method comprising: co-polymerizing N-isopropylacrylamide and N-acryloxysuccinimide with a RAFT agent (e.g., a DMP RAFT agent) to generate a first copolymer comprising an acrylic backbone; contacting the copolymer with an alkyl amine and an alkoxy-polyethylene glycol amine to generate a second copolymer; and contacting the second copolymer with isopropylamine to generate a polymer of formula I:

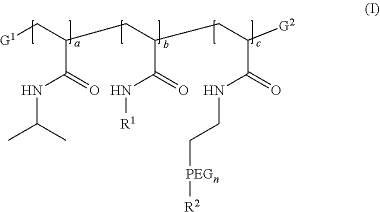

wherein:
a, b and c are molar fractions of the co-monomers, where a, b and c are each greater than zero;
PEGₙ is a polyethyleneglycol polymer and n is an integer from 1 to 2500.
R¹ is an alkyl or a substituted alkyl;
R² is alkyl, substituted alkyl, hetero; alkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl; and
G¹ is

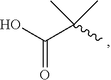

wherein [symbol] is a bond between G¹ and the polymer and G² is

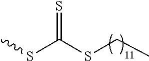

wherein [symbol] is a bond between G² and the polymer.

Aspect 132. A hydrogel composition comprising:
a) a thermoreversible polymer of any one of aspects 1-119; and
b) a buffered aqueous solution.

Aspect 133. The hydrogel composition of aspect 132, further comprising cells.

Aspect 134. The hydrogel composition of aspect 133, wherein the cells are stem cells selected from the group consisting of (a) an adult stem cell derived from bone marrow, umbilical tissues, or placenta; (b) a neural stem cell; (c) a progenitor cell derived from an embryonic stem cell; and (d) embryonic stem cell.

Aspect 135. The hydrogel composition of aspect 134, wherein the cells are mesenchymal stem cells or hematopoietic stem cells.

Aspect 136. The hydrogel composition of aspect 135, wherein the cells are immune cells.

Aspect 137. The hydrogel composition of aspect 136, wherein the immune cells are T cells or natural killer cells.

Aspect 138. The hydrogel composition of any one of aspects 133-137, wherein the cells are genetically modified.

Aspect 140. The hydrogel composition of aspect 137, wherein the cells T cells genetically modified to produce a chimeric antigen receptor.

Aspect 141. A method of growing cells, the method comprising: a) introducing cells into a hydrogel composition of aspect 140 to produce a culturing mixture; and b) incubating the culturing mixture under conditions suitable for growth of the cells.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1: Example Synthesis of Polymer

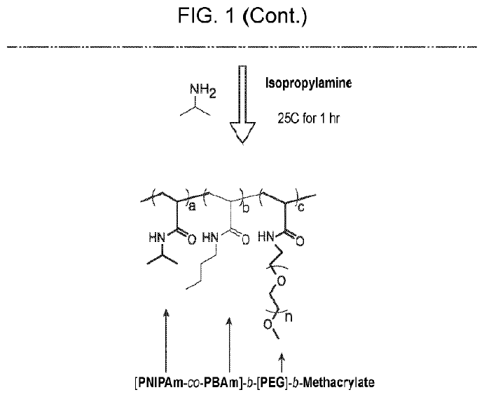

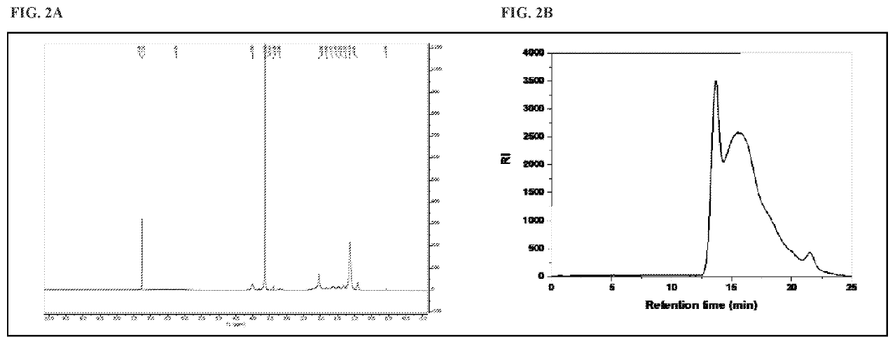

A fully defined and synthetic, thermoreversible, random copolymer based on the interaction of a hydrophilic component (such as poly(ethylene glycol) (PEG)) and temperature sensitive poly(N-isopropylacrylamide) (PNIPAAm) was synthesized. A two-step synthesis process was developed to produce a novel thermoreversible graft copolymer, in which the PEG represents the hydrophilic block, the PNIPAAm represents the hydrophobic block, and the alkyl pendant group (here described as butyl chains but could be any alkyl chain) serves as the temperature shifting moiety (FIG. 1). To generate the thermoreversible graft copolymer, a mixture of NIPAAm and N-acryloxysuccinimide (NASI) was first copolymerized via standard radical polymerization. The resulting functionalizable copolymer, after reprecipitation and drying, was then mixed with an amine-terminated alkyl group (here, butylamine) and a monoamine-terminated PEG block. Both amine-terminated groups attached to the PNIPAAm-co-PNASI backbone via the amine and N-hydroxysuccinimide (NHS) amidation reaction. Finally, the remaining NHS groups were converted to NIPAAm via addition of isopropylamine, and the resulting polymer was dried, dialyzed, and lyophilized. H-NMR characterization of the final polymer (FIG. 2A) demonstrated the presence of the PNIPAAm, PEG, and butylamine. In addition, GPC characterization indicated a polydispersity index (PDI) of 3, with clear lower molecular weight cutoff due to the dialysis (FIG. 2B). The final thermoreversible graft copolymer was then reconstituted in defined cell culture medium at the desired weight percent for further material characterization and cell culture.

Many general references providing commonly known chemical synthetic schemes and conditions useful for synthesizing the disclosed compounds are available (see, e.g., Smith and March, March's Advanced Organic Chemistry: Reactions, Mechanisms, and Structure, Fifth Edition, Wiley-Interscience, 2001; or Vogel, A Textbook of Practical Organic Chemistry, Including Qualitative Organic Analysis, Fourth Edition, New York: Longman, 1978).

Compounds as described herein can be purified by any of the means known in the art, including chromatographic means, such as high performance liquid chromatography (HPLC), preparative thin layer chromatography, flash column chromatography and ion exchange chromatography. Any suitable stationary phase can be used, including normal and reversed phases as well as ionic resins. See, e.g., Introduction to Modern Liquid Chromatography, 2nd Edition, ed. L. R. Snyder and J. J. Kirkland, John Wiley and Sons, 1979; and Thin Layer Chromatography, ed E. Stahl, Springer-Verlag, New York, 1969.

During any of the processes for preparation of the compounds of the present disclosure, it may be necessary and/or desirable to protect sensitive or reactive groups on any of the molecules concerned. This can be achieved by means of conventional protecting groups as described in standard works, such as T. W. Greene and P. G. M. Wuts, “Protective Groups in Organic Synthesis”, Fourth edition, Wiley, New York 2006. The protecting groups can be removed at a convenient subsequent stage using methods known from the art.

The compounds described herein can contain one or more chiral centers and/or double bonds and therefore, can exist as stereoisomers, such as double-bond isomers (i.e., geometric isomers), enantiomers or diastereomers. Accordingly, all possible enantiomers and stereoisomers of the compounds including the stereoisomerically pure form (e.g., geometrically pure, enantiomerically pure or diastereomerically pure) and enantiomeric and stereoisomeric mixtures are included in the description of the compounds herein. Enantiomeric and stereoisomeric mixtures can be resolved into their component enantiomers or stereoisomers using separation techniques or chiral synthesis techniques well known to the skilled artisan. The compounds can also exist in several tautomeric forms including the enol form, the keto form and mixtures thereof. Accordingly, the chemical structures depicted herein encompass all possible tautomeric forms of the illustrated compounds. The compounds described also include isotopically labeled compounds where one or more atoms have an atomic mass different from the atomic mass conventionally found in nature. Examples of isotopes that can be incorporated into the compounds disclosed herein include, but are not limited to, ²H, ³H, ¹¹C, ¹³C, ¹⁴C, ¹⁵N, ¹⁸O, ¹⁷O, etc. Compounds can exist in unsolvated forms as well as solvated forms, including hydrated forms. In general, compounds can be hydrated or solvated. Certain compounds can exist in multiple crystalline or amorphous forms. In general, all physical forms are equivalent for the uses contemplated herein and are intended to be within the scope of the present disclosure.

The nomenclature used herein to name the subject compounds is illustrated in the Examples herein. When possible, this nomenclature has generally been derived using the commercially-available AutoNom software (MDL, San Leandro, Calif.).

An example synthesis is shown in FIG. 1 and FIG. 5.

FIG. 1 presents a schematic depiction of synthesis of a thermoreversible polymer of the present disclosure. Butyl amino is represented; however, a different lower alkyl amine can be substituted. PEG-monoamine is represented as the methoxy group; however, other functional groups can be used. BAm=Butyl Amine; NIPAAm=N-isopropylacrylamide

FIGS. 2A and 2B depict characterization of a thermoreversible polymer as depicted in FIG. 1. FIG. 2A: NMR of poly(NIPAAm-co-BAm)-b-PEG. FIG. 2B: Gel permeation chromatography (GPC) of poly(NIPAAM-co-BAm)-b-PEG.

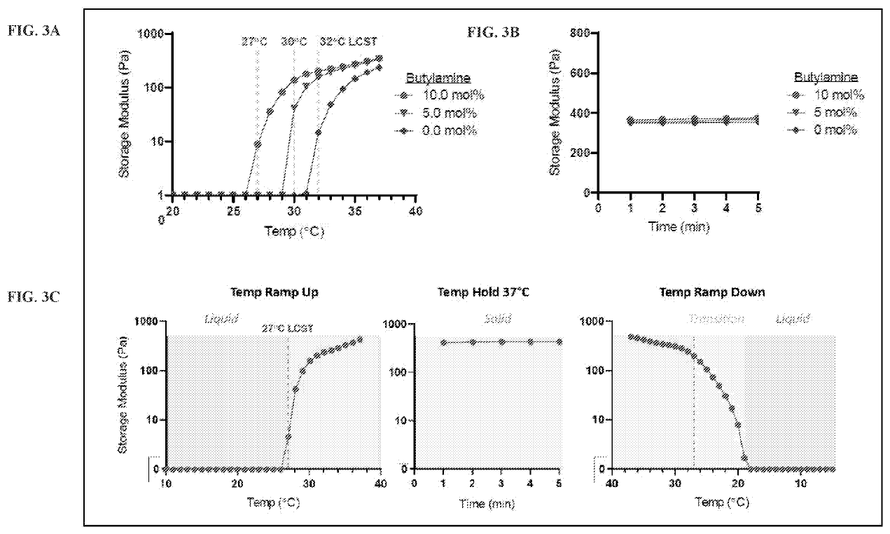

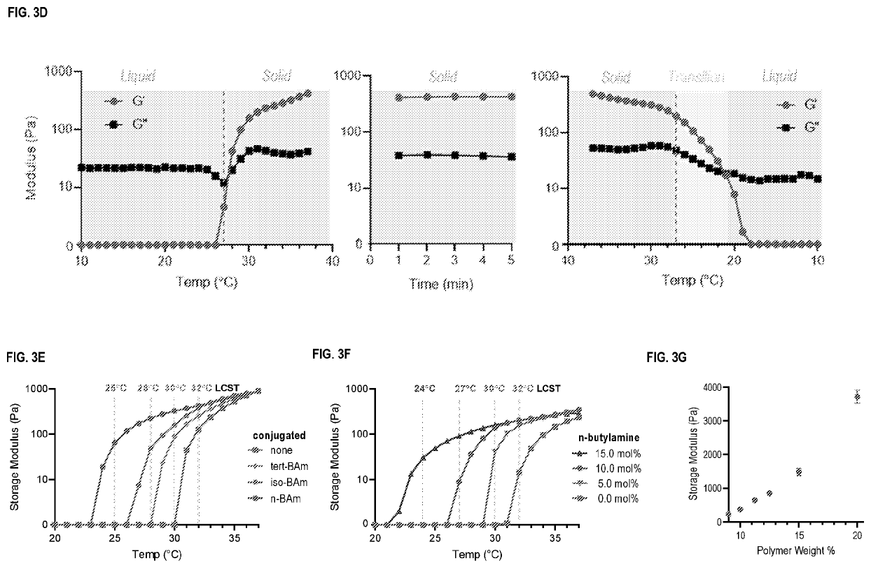

FIG. 3A-3C depict data showing mechanical properties of a thermoreversible polymer of FIG. 1. FIG. 3A: Gelation stiffness vs. temperature of poly(NIPAAm-co-Bam)-b-PEG with varying mol % of BAm. Polymer solutions were tested at 10 wt %. LCST of sol-gel transition is denoted, defined as point of G′>G″. FIG. 3B: Hydrogel stiffness at 37 C. Polymer solution was tested at 10 wt %. FIG. 3C: Thermoreversible properties of the polymer, tested at 10 wt % with 10 mol % BAm.

FIG. 3D-3G depict data showing mechanical properties of a thermoreversible polymer of FIG. 1. FIG. 3D: Thermoreversible properties of a polymer, tested at 10 wt % with 10 mol % BAm. FIG. 3E: Gelation stiffness vs. temperature of poly(NIPAAm-co-BAm)-b-PEG with varying mol % of alkyl amine components, such as n-butyl, tert-butyl, or iso-butyl amine. Polymer solutions were tested at 10 wt %. LCST of sol-gel transition is denoted, defined as point of G′>G″. FIG. 3F: Effect of mole % of n-butyl amine on LCST of the polymer. FIG. 3G: Effect of polymer wt % on resulting hydrogel stiffness at 37 C.

After designing the synthetic strategy for the thermoreversible graft copolymer, key material properties of the hydrogel were investigated. These properties included the thermoreversible gelation via storage and loss modulus of the polymer (FIG. 3D) at various temperatures, the stability of the hydrogel, effect of alkyl amine structure on the thermoreversible polymer LCST (FIG. 3E), effect of alkyl amine amount on the thermoreversible polymer LCST (FIG. 3F) and the effect of polymer wt % on stiffness (FIG. 3G). The addition of the alkyl amine allowed for tight control of LCST in a range of temperatures. Control of the LCST is important to enabled liquid handling and extrusion, and stiffness is important for supporting and controlling cell growth and differentiation, long term stability in an aqueous environment, and reproducible synthesis.

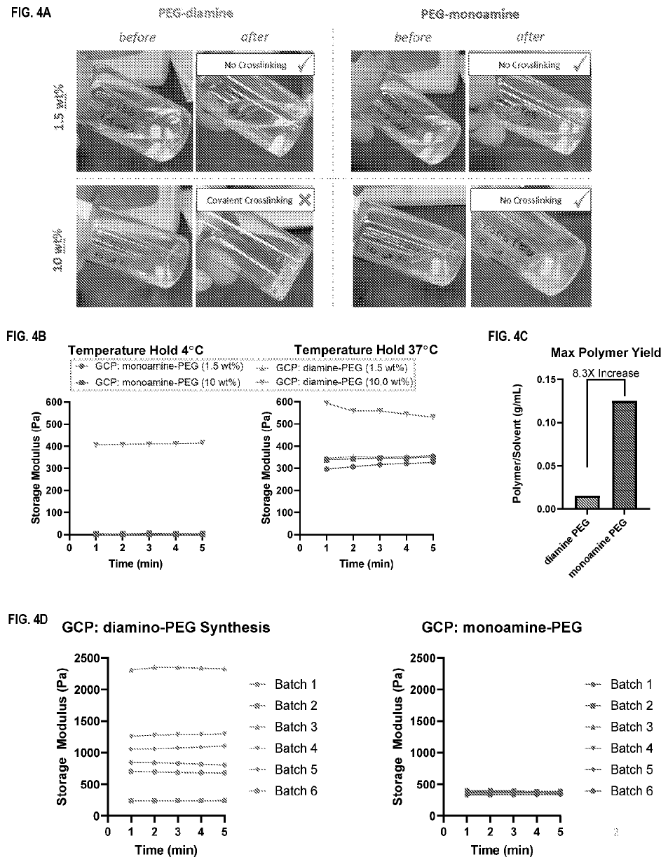

Additionally, the advantage of using a mono-conjugated PEG to graft on the activated PNIPAAm backbone was demonstrated, as this approach prevents any unintended covalent crosslinking that can affect gel integrity (FIG. 4A), where presence of the diaminoPEG to conjugate the hydrophilic group can covalently crosslink at higher reaction concentrations. This covalent crosslinking can prevent the polymer from fully liquifying (FIG. 4B) and influence the final hydrogel stiffness, as it is a mixture of chemical and physical crosslinking (FIG. 4B). Using a monoPEGamine allows for higher reaction yield, as wt % in solvent is not a limiting factor (FIG. 4C). Finally, the use of diaminoPEG results in batch to batch variability in final hydrogel stiffness (FIG. 4D) while using a monoPEGamine results in highly reproducible thermoreversible polymer synthesis.

FIG. 4A-4D depict increased reproducibility and scalability of a thermoreversible polymer of FIG. 1 with PEG-monoamine synthesis. FIG. 4A: Representative images o unwanted covalent crosslinking during polymer synthesis. Representative images of each condition before and after amine/NHS conjugation. Addition of PEGdiamine vs. PEGmonoamine to poly(NIPAAm-co-NASI) reacted in chloroform at room temperature for 24 hours. FIG. 4B: Effect of covalent crosslinking on polymer mechanical properties. FIG. 4C: Maximum polymer yield, determined via reaction concentration without unwanted covalent crosslinking. FIG. 4D. Effect of monoPEGamine on thermoreversible polymer synthesis.

FIG. 5A-5B depict functionalization of a thermoreversible polymer. FIG. 5A: Example synthesis schematic to introduce chemical functionalization sites into the thermoreversible polymer. FIG. 5B: Table describing possible functionalization molecules/structures and bioconjugation methods for the thermoreversible polymer.

### Example 2: Stem Cell Differentiation and Expansion

A thermo hydrogel made with the reversible polymer as described in Example 1 was used to expand and differentiate human pluripotent stem cells (hPSC). The hPSC were induced to differentiate into dopaminergic neurons, cardiomyocytes, or hepatocytes. The hPSCs within the hydrogel were cultured in the media described below, and marker expression was analyzed on day 25 (for dopaminergic neurons), day 15 (for cardiomyocytes), or day 13 (for hepatocytes). Marker expression was analyzed by immunochemistry and flow cytometry. Results were compared with differentiation of hPSCs in the same culture medium but in 2D Matrigel.

### Stem Cell Expansion

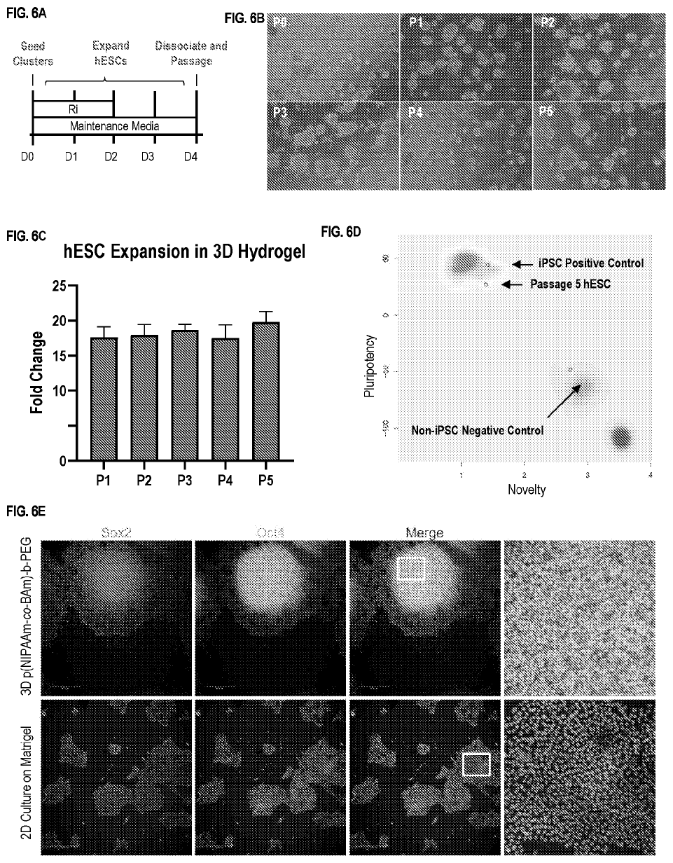

A thermoreversible hydrogel made with the reversible polymer as described in Example 1 was used to expand human pluripotent stem cells (hPSC) in 3D culture over 5 passages. The hPSC were seeded as single cells or clusters in the thermoreversible hydrogel and maintained in stem cell culture medium (Essential 8 or mTeSR) with or without rock inhibitor (Ri; Y-27632) for 4 days (FIG. 6A). The cell aggregates grown within the hydrogel (FIG. 6B) were then collected by cooling/liquifying the hydrogels, the aggregates were singularized and reseeded in the hydrogel, constituting one passage. This process was continued for 5 consecutive passages within the hydrogel. Pluripotency marker expression was analyzed by immunochemistry, flow cytometry, and predicted to remain pluripotent using a commercially available PluriTest.

### Results

hPSCs expanded within the thermoreversible hydrogel exhibited stable cell growth, with ˜20 fold change yield after 4 days of growth over 5 passages (FIG. 6C). The resulting hPSCs after 5 passages within the hydrogel maintained their pluripotency potential, as measured by PluriTest score (FIG. 6D) and pluripotency marker expression, compared to standard 2D culture on Matrigel (FIG. 6E).

### Dopaminergic Neuron Differentiation

For neural induction, hPSCs were grown in the hydrogel described in Example 1, or in 2D Matrigel, in basal medium (DMDM/Neurobasal/N2/B27) containing combinations of LDN, SB, PPA, fibroblast growth factor 8 (FGF8), SHH, and CHIR. “CHIR” IS CHIR99021; 2) “LDN” is LDN-193189; SB is SB431542; and PPA is puromorphine or SAG. For neural specification, the medium was switched to basal medium containing brain-derived neurotrophic factor (BDNF), glial cell-derived neurotrophic factor (GDNF), L-ascorbic acid (LAA), TGF-β, dibutyryl cyclic-AMP (dbCAMP), and the γ-secretase inhibitor N-[N-(3,5-difluorophenacetyl)-L-alanyl]-S-phenylglycine t-butyl ester (DAPT).

Marker expression was analyzed on day 25. The markers analyzed were FOXA2, Tuj1, and tyrosine hydroxylase (TH).

### Cardiomyocyte Differentiation

For cardiac mesoderm induction, hPSCs were grown in the hydrogel described in Example 1, or in 2D Matrigel, in basal medium (RPMI/B27 without insulin) containing CHIR followed by IWP2. For cardiac specification, the medium was changed to basal medium.

Marker expression was analyzed on day 15. The markers analyzed were cardiac troponin t (cTnT), α-actinin (α-Act), and myosin light chain 2 v (MLC2v).

### Hepatocyte Differentiation

For hepatocyte induction, hPSCs were grown in the hydrogel described in Example 1, or in 2D Matrigel, in basal medium (RPMI/B27) containing Activin A and CHIR.

For foregut endoderm specification, the medium was changed to basal medium containing bone morphogenic protein 4 (BMP4) and fibroblast growth factor 2 (FGF2).

For hepatocyte specification, the method was changed to basal medium containing hepatocyte growth factor (HGF).

Marker expression was analyzed on day 13. The markers analyzed were alpha fetoprotein (AFP) and hepatocyte nuclear factor 4 alpha (HNF4α).

### Results

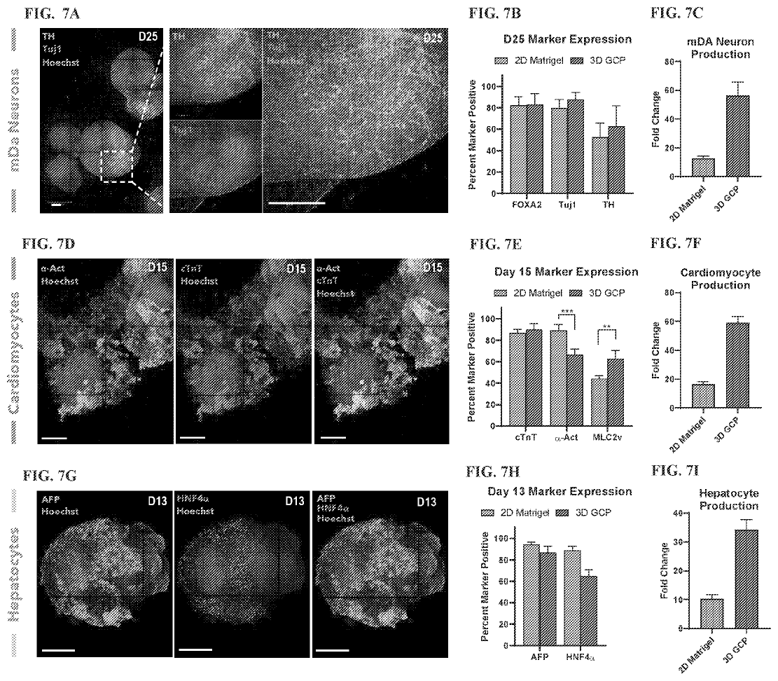

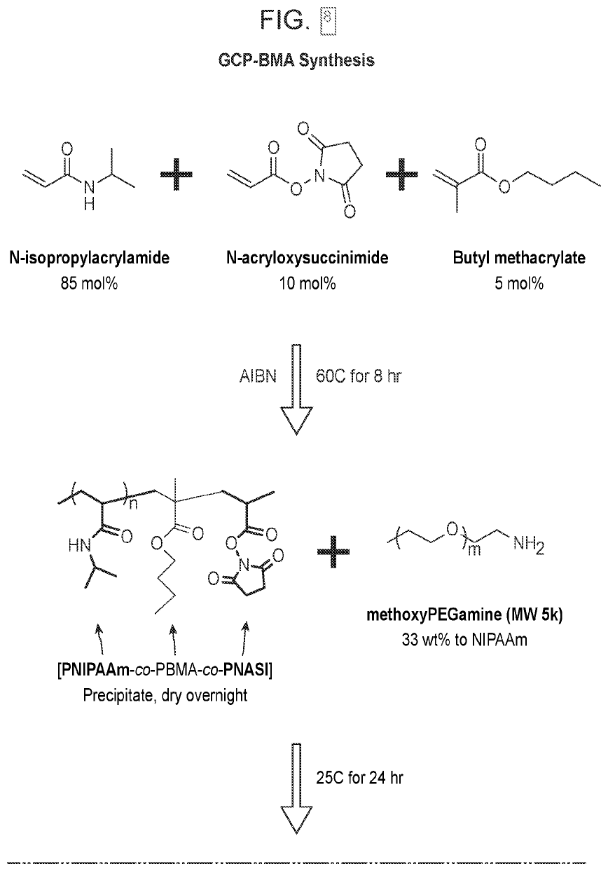

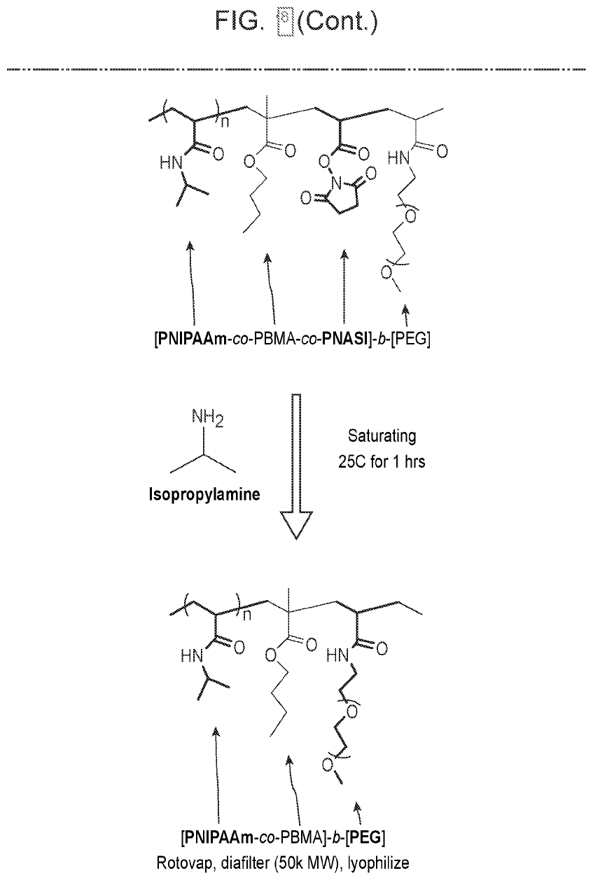

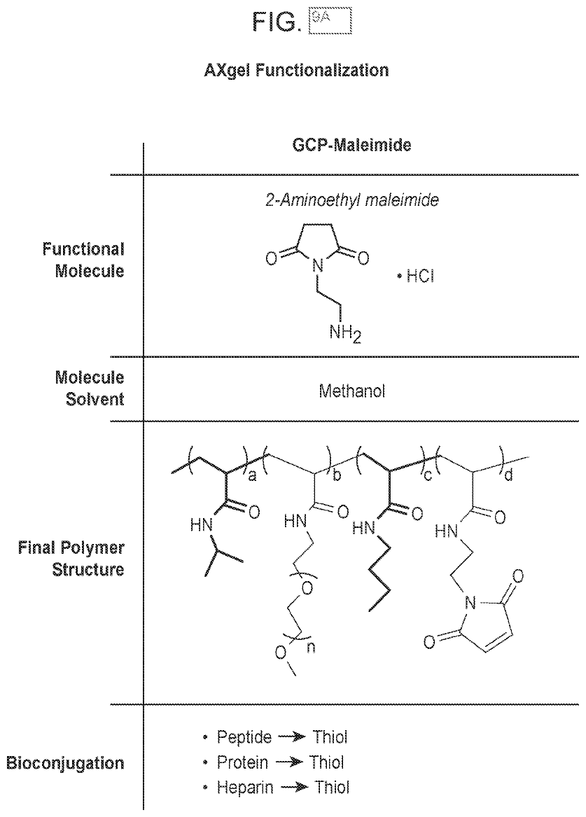

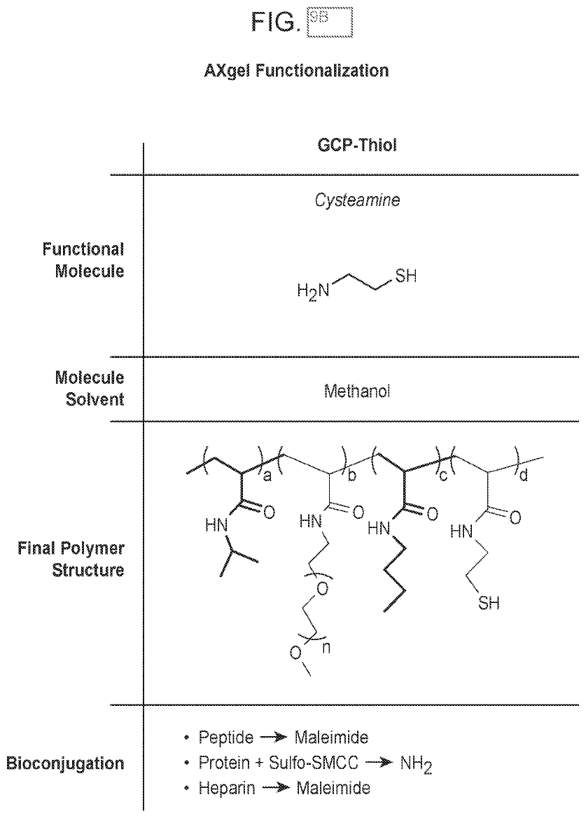

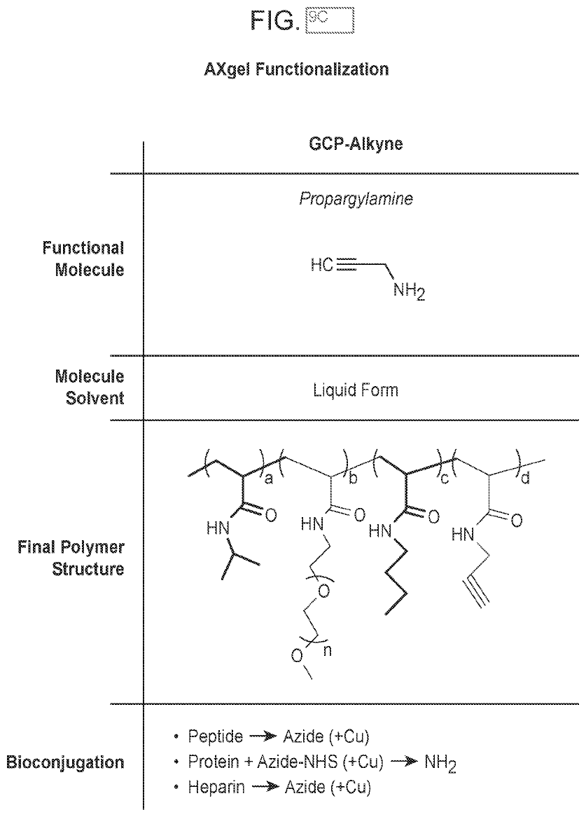

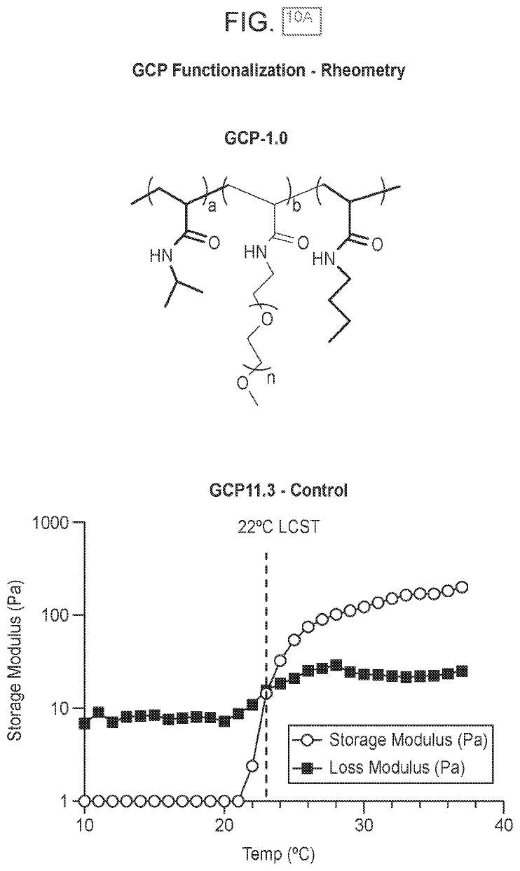

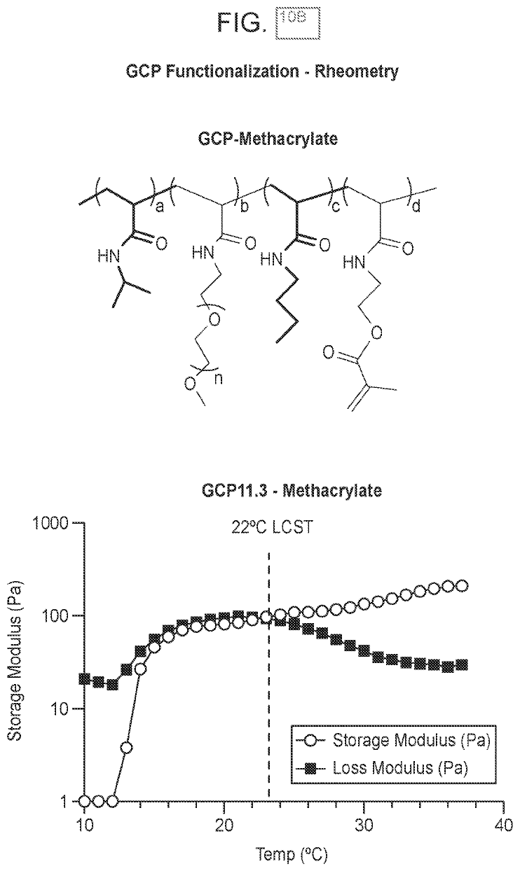

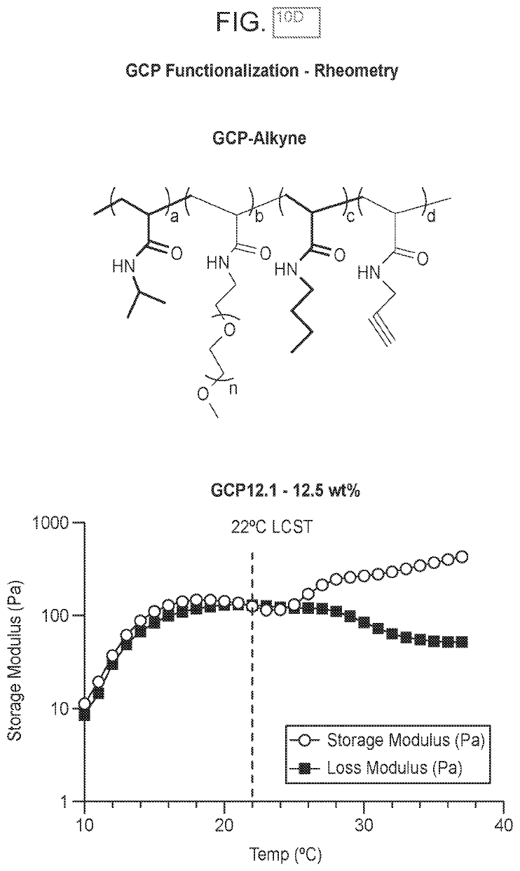

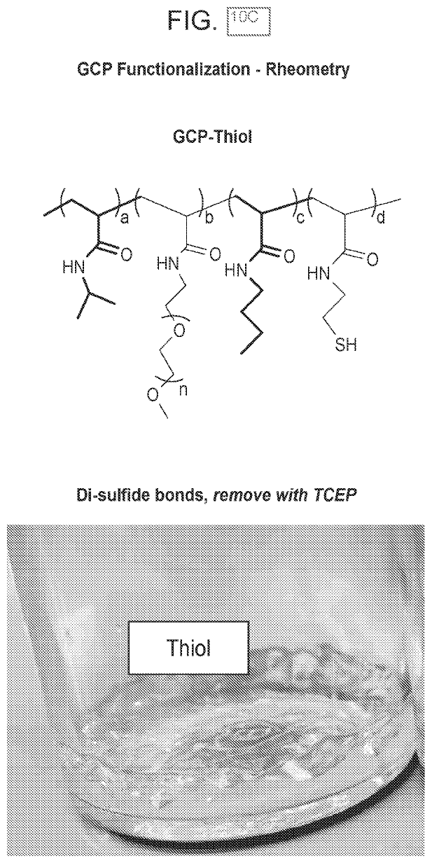

The results are shown in FIG. 7A-7I. Marker expression of cells differentiated in the hydrogel (“3D GCP”) or in Matrigel (“2D Matrigel”) is shown in FIGS. 7B, 7E, and 7H. Production of dopaminergic neurons, cardiomyocytes, and hepatocytes are shown in FIGS. 7C, 7F, and 7I, respectively.

### Example 3: Graft Co-Polymer (GCP) Butyl Methacrylate (BMA) Synthesis and Characterization

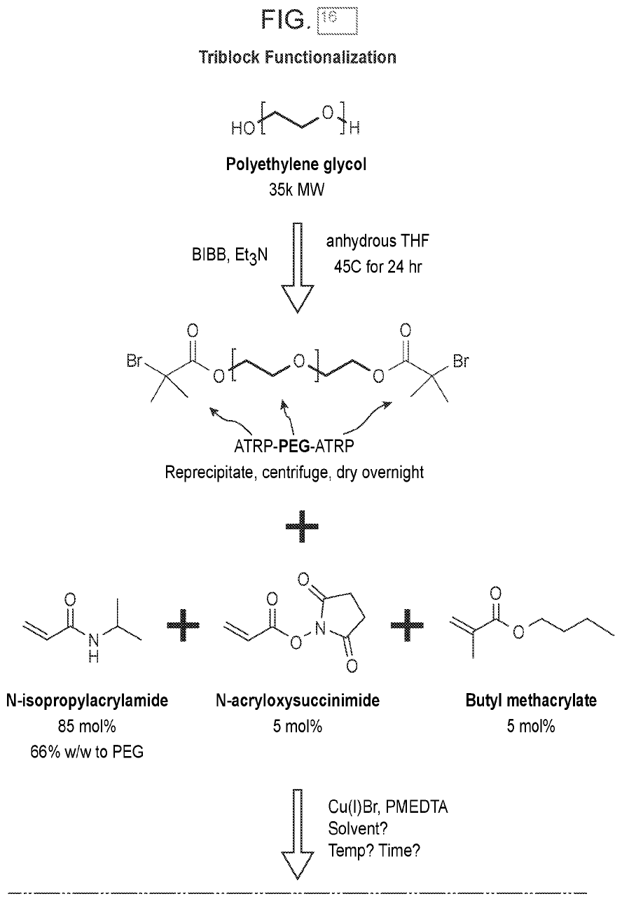

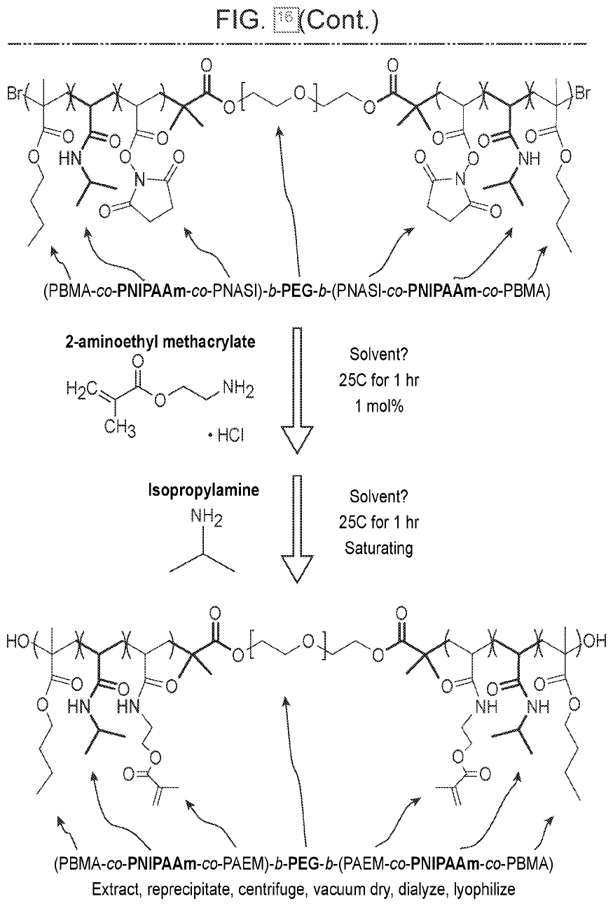

In some situations, a lower viscosity liquid form of the thermoreversible hydrogel may be advantageous, for applications such as liquid handling. A variation of the graft-copolymer was synthesized using an alkyl methacrylate to shift the temperature instead of the second reaction of butyl amine with the free NHS group of NASI. The ability to control temperature is important for downstream cell manufacturing applications, and an alkyl methacrylate provides the ability to shift the hydrophobicity of the polymer backbone lower with less amounts of the temperature shifting component. The use of butyl methacrylate in the first step of the synthesis (FIG. 8), followed by PEG grafting, allowed thermoreversible polymers with similar end storage modulus at lower polymer wt % in solution (FIG. 13A), as compared to the thermoreversible polymer described in FIG. 1 (FIG. 13B).

FIG. 8 depicts synthesis of a the thermoreversible graft copolymer (GCP) made using butyl methacrylate (BMA) as the temperature shifting moiety (GCP-BMA).

FIG. 13A-C present data showing the mechanical properties of GCP-BMA. FIG. 13A: GCP-BMA storage modulus at various wt % in solution compared to the butyl amine (BAm) conjugated thermoreversible graft copolymer (GCP-BAm). FIG. 13B: Matched stiffness of GCP-BMA to GCP-BAm at lower wt % with similar stability. FIG. 13C: Comparison of GCP-BMA at various wt %.

### Example 4: Thermoreversible Polymer Functionalization

Bioconjugation of peptides, protein, and other proteoglycans is an important application for cellular expansion and differentiation. The ability to present or sequester growth factors, cell binding peptides, or chemical modulators of signaling pathways provides microenvironment control that can influence cell fate. To provide bioconjugation sites and schematics to the thermoreversible polymer, 6 independent motifs were devised, which motifs allow for bioconjugation directly to the backbone of the polymer.

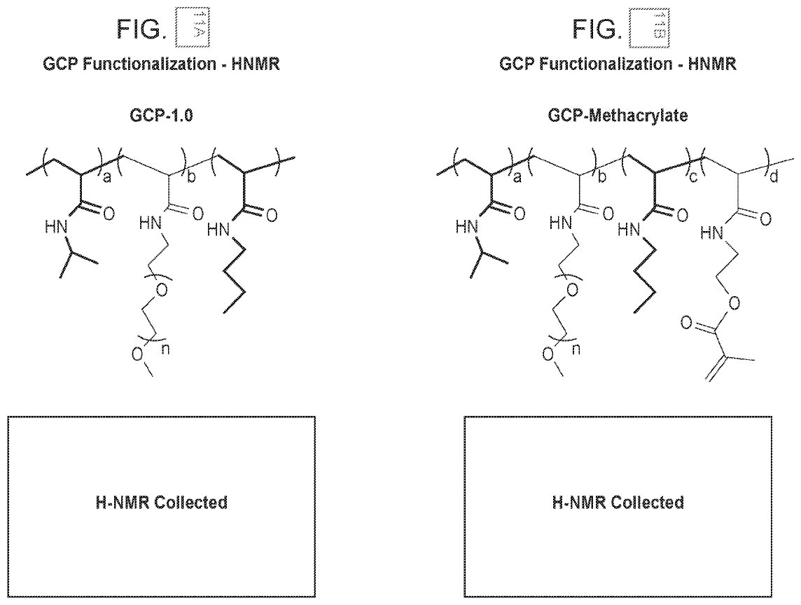

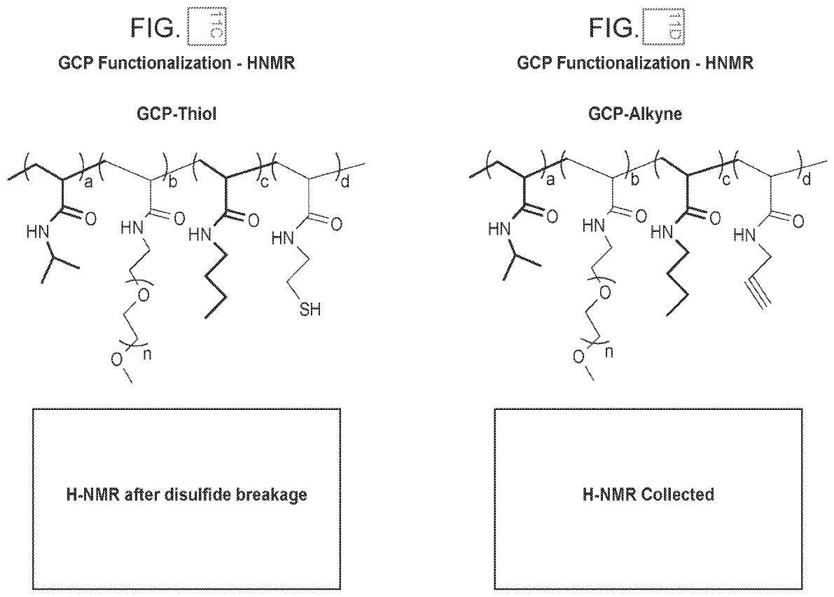

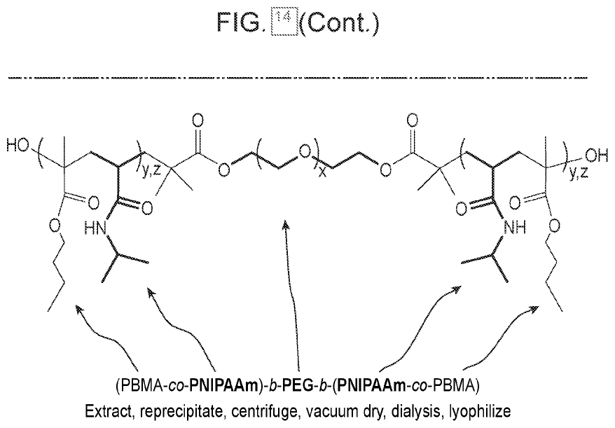

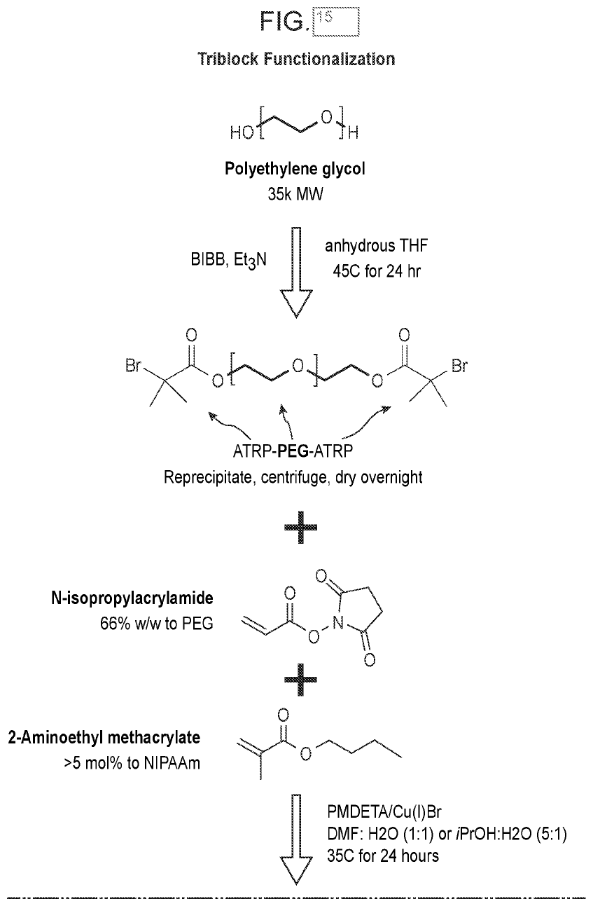

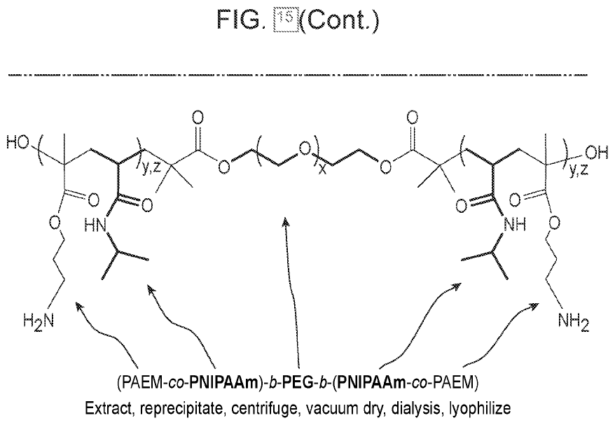

FIG. 9A-9C and FIG. 11A-11D schematically depict functionalization of a GCP. Free methacrylate, thiol, alkyne, carboxylic acid, maleimide, and strained alkyne groups were covalently attached to the polymer backbone reaction of a linked amine to the activated NHS ester present in the backbone before saturation. Each of the respective groups can be leveraged as handle to add proteins, peptides, hyaluronic acid, biotin/streptavidin, heparin, etc. directly to the polymer backbone.

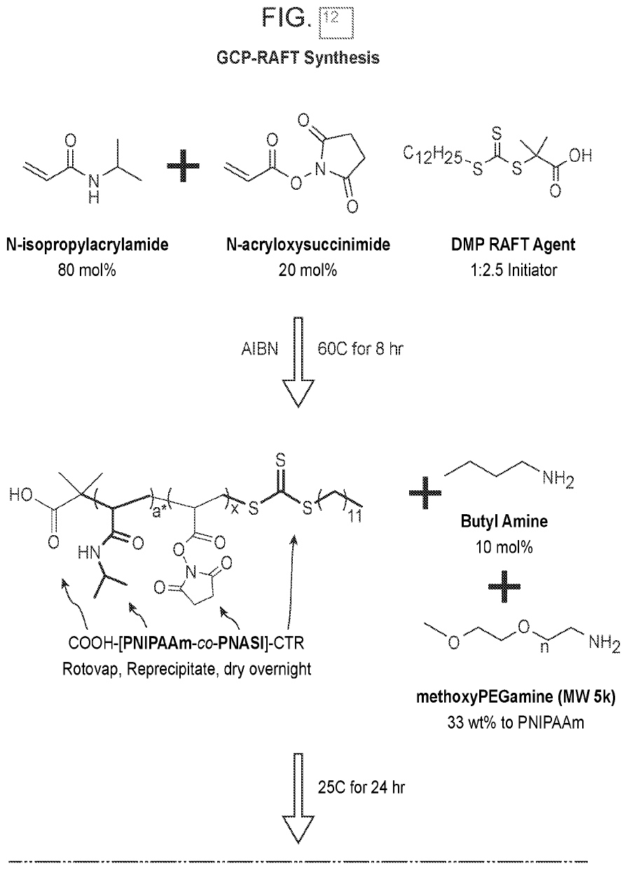

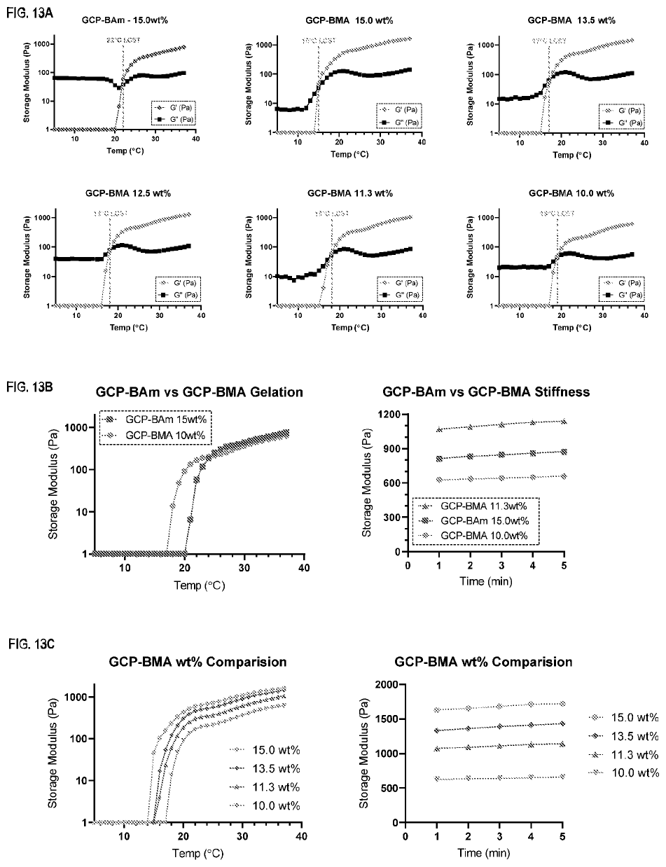

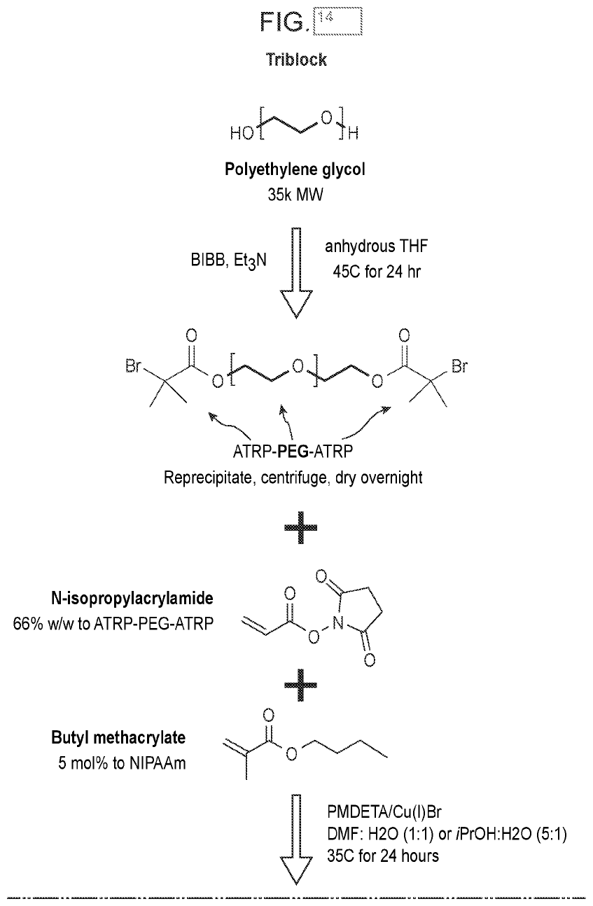

FIG. 10A-10D depict rheometry analysis of functionalized GCPs. Presence of the functional groups at 2 mol % appears to affect the loss modulus, but does not affect final gel structure or stability.

### Example 5: Thermoreversible Polymer Synthesis with RAFT

Typical radical polymerization, with initiators using AIBN, creates polymers of varying polydispersity index. This distribution in polymer length can affect the final mechanical properties of the thermoreversible polymer, including stiffness, LCST, and viscosity. Radical Addition Fragmentation Chain Transfer (RAFT) is a type of living polymerization technique that enables a more controlled addition of monomers to the growing polymer and results in monodisperse polymer lengths. To control the molecular weight of the thermoreversible polymer, a graft copolymer was synthesized in the presence of the RAFT agent 2-(Dodecylthiocarbonothioylthio)-2-methylpropionic acid (DMP). This chain transfer agent, when in the presence of monomers and at a defined ratio to the AIBN initiator, controls the monomer addition to the growing chain and the resulting molecular weight of the polymer. Additionally, the remaining chain transfer reagent can be reduced to a free thiol for use in bioconjugation.

FIG. 12 schematically depicts GCP-RAFT synthesis.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A thermoreversible polymer comprising:
a N-isopropylacrylamide (NIPAM) co-monomer;
a lower alkyl amine co-monomer; and
a poly(ethylene glycol) (PEG) co-monomer, wherein the terminal PEG monomer is substituted with alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl.

2. The thermoreversible polymer of claim 1, wherein:
the lower alkyl amine co-monomer comprises n-butyl, isobutyl, tert-butyl, n-propyl, pentyl, isopropyl, or isopentyl; and
the terminal PEG monomer is substituted with an alkoxy group.

3. The thermoreversible polymer of claim 2, wherein the alkoxy group is an C1-C6 alkoxy selected from the group consisting of methoxy, ethoxy, n-propoxy, isoproxy, n-butoxy, isobutoxy, tert-butoxy, pentoxy and isopentoxy.

4. The thermoreversible polymer of any one of claims 1-3, comprising the formula (I): 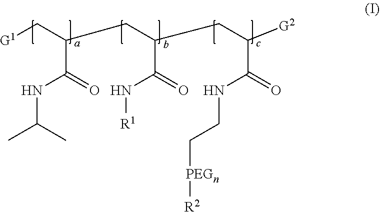
wherein:
a, b, and c are molar fractions of the co-monomers, where a, b, and c are each greater than zero;
PEGₙ is a polyethyleneglycol polymer and n is an integer from 1 to 2500;
R¹ is an alkyl or a substituted alkyl;
R² is alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl; and
G¹ and G² are each independently selected from a polymer segment, a terminal group, a linker and a linked modifying agent.

5. The thermoreversible polymer of claim 4, wherein R¹ is a C1-C6 alkyl.

6. The thermoreversible polymer of claim 5, wherein R¹ is an alkyl group selected from the group consisting of n-butyl, isobutyl, tert-butyl, n-propyl, pentyl, isopropyl and isopentyl.

7. The thermoreversible polymer of claim 6, wherein R¹ is n-butyl.

8. The thermoreversible polymer of any one of claims 4-7, wherein R² is an alkoxy group.

9. The thermoreversible polymer of claim 8, wherein R² is a C1-C6 alkoxy group selected from the group consisting of methoxy, ethoxy, n-propoxy, isoproxy, n-butoxy, isobutoxy, tert-butoxy, pentoxy and isopentoxy.

10. The thermoreversible polymer of claim 9, wherein R² is methoxy.

11. The thermoreversible polymer of any one of claims 2-10, wherein a>0.8; 0.2>b>0; and 0.1>c>0.

12. The thermoreversible polymer of any one of claims 1-11, comprising the formula II: 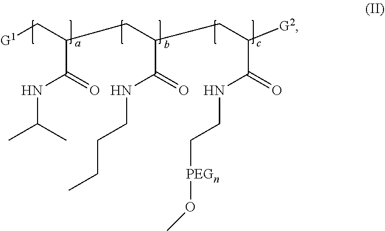
wherein n is 1 to 25; and
G¹ and G² are each independently selected from a polymer segment, a terminal group, a linker and a linked modifying agent.

13. The thermoreversible polymer of any one of claims 1-12, wherein the PEG or PEGₙ has a MW of 2 kDa to 100 kDa.

14. The thermoreversible polymer of any one of claims 1-3, comprising the formula (III): 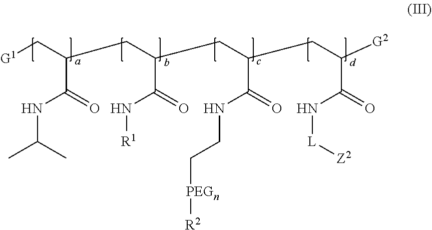
wherein:
a, b, c, and d are molar fractions of the co-monomers, where a, b, c and d are each greater than zero;
PEGₙ is a polyethyleneglycol polymer and n is an integer from 1 to 2500;
R¹ is an alkyl or a substituted alkyl;
R² is alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl;
L is a linker;
Z² is a modifying agent; and
G¹ and G² are each independently selected from a polymer segment, a terminal group, a linker and a linked modifying agent.

15. The thermoreversible polymer of claim 14, wherein R¹ is a C1-C6 alkyl.

16. The thermoreversible polymer of claim 15, wherein R¹ is an alkyl group selected from the group consisting of n-butyl, isobutyl, tert-butyl, n-propyl, pentyl, isopropyl and isopentyl.

17. The thermoreversible polymer of claim 16, wherein R¹ is n-butyl.

18. The thermoreversible polymer of any one of claims 14-17 wherein R² is an alkoxy group.

19. The thermoreversible polymer of claim 18, wherein R² is a C1-C6 alkoxy group selected from the group consisting of methoxy, ethoxy, n-propoxy, isoproxy, n-butoxy, isobutoxy, tert-butoxy, pentoxy and isopentoxy.

20. The thermoreversible polymer of claim 19, wherein R² is methoxy.

21. The thermoreversible polymer of any one of claims 14-20, wherein Z² is a chemoselective functional group selected from a thiol, an alkyne, a cyclooctyne, an azide, a phosphine, a maleimide, an alkoxyamine, an aldehyde and protected versions or precursors thereof.

22. The thermoreversible polymer of any one of claims 14-20, wherein Z² is a modifying agent selected from a heparin, a hyaluronic acid, a specific binding member, a peptide, a nucleic acid, gelatin, fibronectin, collagen, laminin, basis fibroblast growth factor (bFGF), epidermal growth factor (EGF), insulin, progesterone, glucose, stromal cell derived factor-1 (SDF-1), thymosin beta-4, sonic hedgehog (SHH), Noggin, Activin, transforming growth factor-β (TGF-β) (TGFβ3), FGF8, brain-derived neurotrophic factor (BDNF), glial cell-derived neurotrophic factor (GDNF), neurotrophic factor-3 (NT3), platelet-derived growth factor (PDGF), IL-16, IL-2, and insulin-like growth factor-1 (IGF-1).

23. The thermoreversible polymer of any one of claims 2-10, wherein a>0.8; 0.2>b>0; and 0.1>c>0.

24. The thermoreversible polymer of any one of claims 4-23, wherein one or more of G¹, G² and Z² are independently selected a modifying agent selected from a heparin, a hyaluronic acid, a member of a specific binding pair, a polypeptide, and a nucleic acid.

25. The thermoreversible polymer of any one of claims 4-23, wherein one or more of G¹, G² and Z² are independently selected a modifying agent selected from gelatin, fibronectin, collagen, or laminin.

26. The thermoreversible polymer of any one of claims 4-23, wherein one or more of G¹, G² and Z² is a polypeptide selected from a chemokine, a peptide hormone, or a growth factor.

27. The thermoreversible polymer of claim 26, wherein the polypeptide is fibroblast growth factor, epidermal growth factor, hepatic growth factor insulin, stromal cell-derived factor-1, thymosin beta-4, sonic hedgehog, Noggin, activin, transforming growth factor, bone morphogenic protein, brain-derived neurotrophic factor, glial cell-derived neurotrophic factor, neurotrophin-3, platelet-derived growth factor, FGF-2, FGF-8, keratinocyte growth factor, or insulin-like growth factor.

28. The thermoreversible polymer of claim 26, wherein the polypeptide is selected from hepatic growth factor; bone morphogenic protein; FGF-2; FGF-8, and keratinocyte growth factor.

29. The thermoreversible polymer of any one of claims 1-28, wherein the thermoreversible polymer is a solid at 20° C. or more.

30. The thermoreversible polymer of any one of claims 1-28, wherein the thermoreversible polymer is a solid at 37° C.

31. The thermoreversible polymer of any one of claims 1-28, wherein the thermoreversible polymer is a liquid at 30° C. or less.

32. The thermoreversible polymer of any one of claims 1-31, wherein the thermoreversible polymer is a liquid at 4° C.

33. A method of making a thermoreversible polymer, the method comprising:
co-polymerizing N-isopropylacryliamide and N-acryloxysuccinimide to generate a first copolymer comprising an acrylic backbone;
contacting the copolymer with an alkyl amine and an alkoxy-polyethylene glycol amine to generate a second copolymer; and
contacting the second copolymer with isopropylamine to generate a polymer of formula I: 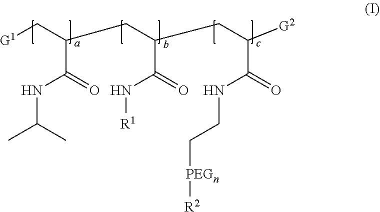
wherein:
a, b and c are molar fractions of the co-monomers, where a, b and c are each greater than zero;
PEGₙ is a polyethyleneglycol polymer and n is an integer from 1 to 2500.
R¹ is an alkyl or a substituted alkyl;
R² is alkyl, substituted alkyl, hetero; alkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl; and
G¹ and G² are each independently selected from a polymer segment, a terminal group, a linker and a linked modifying agent.

34. The method of claim 33, wherein R¹ is a C1-C6 alkyl.

35. The method of claim 34, wherein R¹ is an alkyl group selected from the group consisting of n-butyl, isobutyl, tert-butyl, n-propyl, pentyl, isopropyl and isopentyl.

36. The method of claim 34, wherein R¹ is n-butyl.

37. The method of any one of claims 33-36, wherein R² is an alkoxy group.

38. The method of claim 37, wherein R² is a C1-C6 alkoxy group selected from the group consisting of methoxy, ethoxy, n-propoxy, isoproxy, n-butoxy, isobutoxy, tert-butoxy, pentoxy and isopentoxy.

39. The method of claim 28, wherein R² is methoxy.

40. The method of any one of claims 33-39, wherein a>0.8; 0.1>b>0; and 0.2>c>0.

41. The method of any one of claims 33-40, wherein the method comprises:
co-polymerizing N-isopropylacrylamide and N-acryloxysuccinimide to generate a first copolymer comprising an acrylic backbone;
contacting the copolymer with butylamine and methoxy-polyethylene glycol amine to generate a second copolymer; and
contacting the second copolymer with isopropylamine to generate a polymer of formula II: 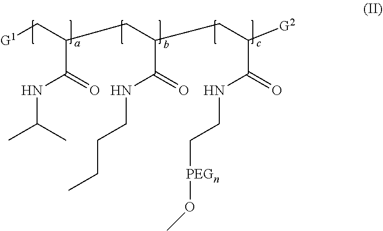
wherein n is an integer from 1 to 2500; and
G¹ and G² are each independently selected from a polymer segment, a terminal group, a linker and a linked modifying agent.

42. A hydrogel composition comprising:
a) a thermoreversible polymer of any one of claims 1-41; and
b) a buffered aqueous solution.

43. The hydrogel composition of claim 42, further comprising cells.

44. The hydrogel composition of claim 43, wherein the cells are stem cells selected from the group consisting of (a) an adult stem cell derived from bone marrow, umbilical tissues, or placenta; (b) a neural stem cell; (c) a progenitor cell derived from an embryonic stem cell; and (d) embryonic stem cell.

45. The hydrogel composition of claim 44, wherein the cells are mesenchymal stem cells or hematopoietic stem cells.

46. The hydrogel composition of claim 43, wherein the cells are immune cells.

47. The hydrogel composition of claim 46, wherein the immune cells are T cells or natural killer cells.

48. The hydrogel composition of any one of claims 43-47, wherein the cells are genetically modified.

49. The hydrogel composition of claim 47, wherein the cells T cells genetically modified to produce a chimeric antigen receptor.

50. A method of growing cells, the method comprising:
introducing cells into a hydrogel composition of claim 42 to produce a culturing mixture; and
incubating the culturing mixture under conditions suitable for growth of the cells.
